SEQUENCE LISTING (92 sequences)

SEQ ID NO: 1 - protein, 120 aa, Artificial
  Synthetic - Humanized 3G9 Heavy Chain
EVQLVESGGGLVQPGGSLRLSCAASGFTFSRYVMSWVRQAPGKGLEWVASISSGGRMYYPDTVKGRFTISRDNAKNSLYLQMNSLRAEDTAVYYCARGSIYDGYYVFPYWGQGTLVTVSS

SEQ ID NO: 2 - protein, 108 aa, Artificial
  Synthetic - Humanized 3G9 Light Chain
EIVLTQSPATLSLSPGERATLSCSASSSVSSSYLYWYQQKPGQAPRLLIYSTSNLASGIPARFSGSGSGTDFTLTISSLEPEDFAVYYCHQWSTYPPTFGGGTKVEIK

SEQ ID NO: 3 - protein, 125 aa, Artificial
  Synthetic - Humanized 8G6 Heavy Chain
QVQLVQSGAEVKKPGASVKVSCKASGYTFDYAMHWVRQAPGQGLEWMGVISTYYGNTNYNQKFKGRVTMTRDTSISTAYMELSRLRSDDTAVYYCARGGLRRGDRPSLRYAMDYWGQGTLVTVSS

SEQ ID NO: 4 - protein, 111 aa, Artificial
  Synthetic - Humanized 8G6 Light Chain
EIVLTQSPATLSLSPGERATLSCRASQSVSTSSYSYMYWYQQKPGQAPRLLIYYASNLESGIPARFSGSGSGTDFTLTISSLEPEDFAVYYCQHNWEIPFTFGGGTKVEIK

SEQ ID NO: 5 - DNA, 711 bp, Artificial
  Synthetic - pKJS195 vector - 3G9 version 5 light chain
atggacttccaggtgcagatcttcagcttcctgctgatcagcgtgagcgtgatcatgagccgcggcgagatcgtgctgacccagagccccgccaccctgagcctgagccccggcgagagggccaccctgagctgcagcgccagcagcagcgtgagcagcagctacctgtactggtaccagcagaagcccggccaggcccccaggctgctgatctacagcaccagcaacctggccagcggcatccccgcccgcttcagcggcagcggcagcggcaccgacttcaccctgaccatcagcagcctggagcccgaggacttcgccgtgtactactgccaccagtggagcacctacccccccaccttcggcggcggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 6 - DNA, 1404 bp, Artificial
  Synthetic - pKJS189 vector - 3G9 vector 3 heavy chain
atggacttcggcctgagctgggtgttcctggtgctggtgctgaagggcgtgcagtgcgaggtgcagctggtggagagcggcggcggcctggtgcagcccggcggcagcctgaggctgagctgcgccgccagcggcttcaccttcagccgctacgtgatgagctgggtgcgccaggcccccggcaagggcctggagtgggtggccagcatcagcagcggaggccgcatgtactaccccgacaccgtgaagggccgcttcaccatcagccgcgacaacgccaagaacagcctgtacctgcagatgaacagcctgcgcgccgaggacaccgccgtgtactactgcgcccgcggcagcatctacgacggctactacgtgttcccctactggggccagggcaccctggtgaccgtgagctccgccagcaccaagggccccagcgtgttccccctggcccccagcagcaagagcaccagcggcggcaccgccgccctgggctgcctggtgaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggt

SEQ ID NO: 7 - DNA, 1404 bp, Artificial
  Synthetic - pKJS196 vector - aglycosyl-3G9 version 3 heavy chain
atggacttcggcctgagctgggtgttcctggtgctggtgctgaagggcgtgcagtgcgaggtgcagctggtggagagcggcggcggcctggtgcagcccggcggcagcctgaggctgagctgcgccgccagcggcttcaccttcagccgctacgtgatgagctgggtgcgccaggcccccggcaagggcctggagtgggtggccagcatcagcagcggaggccgcatgtactaccccgacaccgtgaagggccgcttcaccatcagccgcgacaacgccaagaacagcctgtacctgcagatgaacagcctgcgcgccgaggacaccgccgtgtactactgcgcccgcggcagcatctacgacggctactacgtgttcccctactggggccagggcaccctggtgaccgtgagctccgccagcaccaagggccccagcgtgttccccctggcccccagcagcaagagcaccagcggcggcaccgccgccctgggctgcctggtgaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggt

SEQ ID NO: 8 - protein, 111 aa, Artificial
  Synthetic -hu8G6 version 1 light chain
DIVLTQSPATLSLSPGERATLSCRASQSVSTSSYSYMYWYQQKPGQAPRFLIKYASNLESGIPARFSGSGSGTDFTLTISSLEPEDFAVYYCQHNWEIPFTFGGGTKVEIK

SEQ ID NO: 9 - protein, 111 aa, Artificial
  Synthetic - hu8G6 version 2 light chain
EIVLTQSPATLSLSPGERATLSCRASQSVSTSSYSYMYWYQQKPGQAPRFLIKYASNLESGIPARFSGSGSGTDFTLTISSLEPEDFAVYYCQHNWEIPFTFGGGTKVEIK

SEQ ID NO: 10 - protein, 111 aa, Artificial
  Synthetic - hu8G6 version 3 light chain
EIVLTQSPATLSLSPGERATLSCRASQSVSTSSYSYMYWYQQKPGQAPRLLIKYASNLESGIPARFSGSGSGTDFTLTISSLEPEDFAVYYCQHNWEIPFTFGGGTKVEIK

SEQ ID NO: 11 - protein, 126 aa, Artificial
  Synthetic - hu8G6 version 1 heavy chain
QVQLVQSGAEVKKPGASVKVSCKGSSYTFTDYAMHWVRLAPGQGLEWIGVISTYYGNTNYNQKFKGRATMTVDKSISTAYMELSRLRSDDTAVYYCARGGLRRGDRPSLRYAMDYWGQGTLVTVSS

SEQ ID NO: 12 - protein, 126 aa, Artificial
  Synthetic - hu8G6 version 2 heavy chain
QVQLVQSGAEVKKPGASVKVSCKASGYTFTDYAMHWVRQAPGQGLEWIGVISTYYGNTNYNQKFKGRATMTVDKSISTAYMELSRLRSDDTAVYYCARGGLRRGDRPSLRYAMDYWGQGTLVTVSS

SEQ ID NO: 13 - protein, 126 aa, Artificial
  Synthetic - hu8G6 version 3 heavy chain
QVQLVQSGAEVKKPGASVKVSCKASGYTFTDYAMHWVRQAPGQGLEWMGVISTYYGNTNYNQKFKGRATMTVDKSISTAYMELSRLRSDDTAVYYCARGGLRRGDRPSLRYAMDYWGQGTLVTVSS

SEQ ID NO: 14 - protein, 10 aa, Artificial
  Synthetic - Heavy chain CDR1 sequence for 8G6 antibody
SYTFTDYAMH

SEQ ID NO: 15 - protein, 10 aa, Artificial
  Synthetic - Heavy chain CDR1 sequence for 1A8 antibody
SYTFTDYTMH

SEQ ID NO: 16 - protein, 10 aa, Artificial
  Synthetic - Heavy chain CDR1 sequence for 2B1 and 3G9 antibodies
GFTFSRYVMS

SEQ ID NO: 17 - protein, 10 aa, Artificial
  Synthetic - Heavy chain CDR1 sequence for 2A1 antibody
GYDFNNDLIE

SEQ ID NO: 18 - protein, 10 aa, Artificial
  Synthetic - Heavy chain CDR1 sequence for 2G2 antibody
GYAFTNYLIE

SEQ ID NO: 19 - protein, 17 aa, Artificial
  Synthetic -  Heavy chain CDR2 sequence for 8G6 antibody
VISTYYGNTNYNQKFKG

SEQ ID NO: 20 - protein, 17 aa, Artificial
  Synthetic - Heavy chain CDR2 sequence for 1A8 antibody
VIDTYYGKTNYNQKFEG

SEQ ID NO: 21 - protein, 16 aa, Artificial
  Synthetic - Heavy chain CDR2 sequence for 2B1 antibody
SISSGGSTYYPDSVKG

SEQ ID NO: 22 - protein, 16 aa, Artificial
  Synthetic - Heavy chain CDR2 sequence for 3G9 antibody
SISSGGRMYYPDTVKG

SEQ ID NO: 23 - protein, 17 aa, Artificial
  Synthetic - Heavy chain CDR2 sequence for 2A1 antibody
VINPGSGRTNYNEKFKG

SEQ ID NO: 24 - protein, 17 aa, Artificial
  Synthetic - Heavy chain CDR2 sequence for 2G2 antibody
VISPGSGIINYNEKFKG

SEQ ID NO: 25 - protein, 17 aa, Artificial
  Synthetic -  Heavy chain CDR3 sequence for 8G6 antibody
GGLRRGDRPSLRYAMDY

SEQ ID NO: 26 - protein, 17 aa, Artificial
  Synthetic - Heavy chain CDR3 sequence for 1A8 antibody
GGFRRGDRPSLRYAMDS

SEQ ID NO: 27 - protein, 12 aa, Artificial
  Synthetic - Heavy chain CDR3 sequence for 2B1 antibody
GAIYDGYYVFAY

SEQ ID NO: 28 - protein, 12 aa, Artificial
  Synthetic - Heavy chain CDR3 sequence for 3G9 antibody
GSIYDGYYVFPY

SEQ ID NO: 29 - protein, 12 aa, Artificial
  Synthetic -  Heavy chain CDR3 sequence for 2A1 antibody
IYYGPHSYAMDY

SEQ ID NO: 30 - protein, 11 aa, Artificial
  Synthetic - Heavy chain CDR3 sequence for 2G2 antibody
IDYSGPYAVDD

SEQ ID NO: 31 - protein, 15 aa, Artificial
  Synthetic -  Light chain CDR1 sequence for 8G6 antibody
RASQSVSTSSYSYMY

SEQ ID NO: 32 - protein, 15 aa, Artificial
  Synthetic - Light chain CDR1 sequence for 1A8 antibody
RASQSVSISTYSYIH

SEQ ID NO: 33 - protein, 12 aa, Artificial
  Synthetic - Light chain CDR1 sequence for 2B1 antibody
SASSSVSSSYLY

SEQ ID NO: 34 - protein, 12 aa, Artificial
  Synthetic - Light chain CDR1 sequence for 3G9 antibody
SANSSVSSSYLY

SEQ ID NO: 35 - protein, 11 aa, Artificial
  Synthetic - Light chain CDR1 sequence for 2A1 antibody
KASLDVRTAVA

SEQ ID NO: 36 - protein, 11 aa, Artificial
  Synthetic - Light chain CDR1 sequence for 2G2 antibody
KASQAVNTAVA

SEQ ID NO: 37 - protein, 7 aa, Artificial
  Synthetic - Light chain CDR2 sequence for 8G6 and 1A8 antibodies
YASNLES

SEQ ID NO: 38 - protein, 7 aa, Artificial
  Synthetic - Light chain CDR1 sequence for 2B1 and 3G9 antibodies
STSNLAS

SEQ ID NO: 39 - protein, 7 aa, Artificial
  Synthetic - Light chain CDR2 sequence for 2A1 antibody
SASYRYT

SEQ ID NO: 40 - protein, 7 aa, Artificial
  Synthetic - Light chain CDR2 sequence for 2G2 antibody
SASYQYT

SEQ ID NO: 41 - protein, 9 aa, Artificial
  Synthetic -Light chain CDR3 sequence for 8G6 antibody
QHNWEIPFT

SEQ ID NO: 42 - protein, 9 aa, Artificial
  Synthetic -Light chain CDR3 sequence for 1A8 antibody
QHSWEIPYT

SEQ ID NO: 43 - protein, 9 aa, Artificial
  Synthetic - Light chain CDR3 sequence for 2B1 antibody
HQWSSYPPT

SEQ ID NO: 44 - protein, 9 aa, Artificial
  Synthetic - Light Chain CDR3 sequence for 3G9 antibody
HQWSTYPPT

SEQ ID NO: 45 - protein, 9 aa, Artificial
  Synthetic - Light chain CDR3 sequence for 2A1 antibody
QQHYGIPWT

SEQ ID NO: 46 - protein, 9 aa, Artificial
  Synthetic - Light chain CDR3 sequence for 2G2 antibody
QHHYGVPWT

SEQ ID NO: 47 - DNA, 324 bp, Artificial
  Synthetic - hu3G9 version 1 light chain
gagatcgtgctgacccagagccccgccaccctgagcctgagccccggcgagagggccaccctgagctgcagcgccagcagcagcgtgagcagcagctacctgtactggtaccagcagaagcccggccaggcccccaggctgtggatctacagcaccagcaacctggccagcggcgtgcccgtgcgcttcagcggcagcggcagcggcaccgacttcaccctgaccatcagcagcctggagcccgaggacttcgccgtgtacttctgccaccagtggagcacctacccccccaccttcggcggcggcaccaaggtggagatcaag

SEQ ID NO: 48 - DNA, 324 bp, Artificial
  Synthetic - hu3G9 version 2 light chain
gagatcgtgctgacccagagccccgccaccctgagcctgagccccggcgagagggccaccctgagctgcagcgccagcagcagcgtgagcagcagctacctgtactggtaccagcagaagcccggccaggcccccaggctgtggatctacagcaccagcaacctggccagcggcgtgcccgcccgcttcagcggcagcggcagcggcaccgacttcaccctgaccatcagcagcctggagcccgaggacttcgccgtgtactactgccaccagtggagcacctacccccccaccttcggcggcggcaccaaggtggagatcaag

SEQ ID NO: 49 - DNA, 324 bp, Artificial
  Synthetic - hu3G9 version 3 light chain
gagatcgtgctgacccagagccccgccaccctgagcctgagccccggcgagagggccaccctgagctgcagcgccagcagcagcgtgagcagcagctacctgtactggtaccagcagaagcccggccaggcccccaggctgtggatctacagcaccagcaacctggccagcggcatccccgcccgcttcagcggcagcggcagcggcaccgacttcaccctgaccatcagcagcctggagcccgaggacttcgccgtgtactactgccaccagtggagcacctacccccccaccttcggcggcggcaccaaggtggagatcaag

SEQ ID NO: 50 - DNA, 324 bp, Artificial
  Synthetic - hu3G9 version 4 light chain
cagatcgtgctgacccagagccccgccaccctgagcctgagccccggcgagagggccaccctgagctgcagcgccagcagcagcgtgagcagcagctacctgtactggtaccagcagaagcccggccaggcccccaggctgtggatctacagcaccagcaacctggccagcggcatccccgcccgcttcagcggcagcggcagcggcaccgacttcaccctgaccatcagcagcctggagcccgaggacttcgccgtgtactactgccaccagtggagcacctacccccccaccttcggcggcggcaccaaggtggagatcaag

SEQ ID NO: 51 - DNA, 324 bp, Artificial
  Synthetic - hu3G9 version 5 light chain
gagatcgtgctgacccagagccccgccaccctgagcctgagccccggcgagagggccaccctgagctgcagcgccagcagcagcgtgagcagcagctacctgtactggtaccagcagaagcccggccaggcccccaggctgctgatctacagcaccagcaacctggccagcggcatccccgcccgcttcagcggcagcggcagcggcaccgacttcaccctgaccatcagcagcctggagcccgaggacttcgccgtgtactactgccaccagtggagcacctacccccccaccttcggcggcggcaccaaggtggagatcaag

SEQ ID NO: 52 - DNA, 360 bp, Artificial
  Synthetic -  hu3G9 version 1 heavy chain
gaggtgatgctggtggagagcggcggcggcctggtgcagcccggcggcagcctgaggctgagctgcgccgccagcggcttcaccttcagccgctacgtgatgagctgggtgcgccaggcccccggcaagggcctggagtgggtggccagcatcagcagcggaggccgcatgtactaccccgacaccgtgaagggccgcttcaccatcagccgcgacagcgccaagaacagcctgtacctgcagatgaacagcctgcgcgccgaggacaccgccgtgtactactgcgcccgcggcagcatctacgacggctactacgtgttcccctactggggccagggcaccctggtgaccgtgagctcc

SEQ ID NO: 53 - DNA, 360 bp, Artificial
  Synthetic - hu3G9 version 2 heavy chain
gaggtgcagctggtggagagcggcggcggcctggtgcagcccggcggcagcctgaggctgagctgcgccgccagcggcttcaccttcagccgctacgtgatgagctgggtgcgccaggcccccggcaagggcctggagtgggtggccagcatcagcagcggaggccgcatgtactaccccgacaccgtgaagggccgcttcaccatcagccgcgacagcgccaagaacagcctgtacctgcagatgaacagcctgcgcgccgaggacaccgccgtgtactactgcgcccgcggcagcatctacgacggctactacgtgttcccctactggggccagggcaccctggtgaccgtgagctcc

SEQ ID NO: 54 - DNA, 360 bp, Artificial
  Synthetic - hu3G9 versions 3 and 5 heavy chain
gaggtgcagctggtggagagcggcggcggcctggtgcagcccggcggcagcctgaggctgagctgcgccgccagcggcttcaccttcagccgctacgtgatgagctgggtgcgccaggcccccggcaagggcctggagtgggtggccagcatcagcagcggaggccgcatgtactaccccgacaccgtgaagggccgcttcaccatcagccgcgacaacgccaagaacagcctgtacctgcagatgaacagcctgcgcgccgaggacaccgccgtgtactactgcgcccgcggcagcatctacgacggctactacgtgttcccctactggggccagggcaccctggtgaccgtgagctcc

SEQ ID NO: 55 - protein, 97 aa, Artificial
  Synthetic - murine heavy chain sequence for hu3G9
EVMLVESGGGLVKPGGSLKLSCAASGFTFSRYVMSWVRQTPEKRLEWVASISSGGRMYYPDTVKGRFTISRDSARNILYLQMSSLRSEDTAMYYCAR

SEQ ID NO: 56 - protein, 97 aa, Artificial
  Synthetic - 3G9HV1 heavy chain sequence for hu3G9
EVMLVESGGGLVQPGGSLRLSCAASGFTFSRYVMSWVRQAPGKGLEWVASISSGGRMYYPDTVKGRFTISRDSAKNSLYLQMNSLRAEDTAVYYCAR

SEQ ID NO: 57 - protein, 97 aa, Artificial
  Synthetic - 3G9HV2 heavy chain sequence for hu3G9
EVQLVESGGGLVQPGGSLRLSCAASGFTFSRYVMSWVRQAPGKGLEWVASISSGGRMYYPDTVKGRFTISRDSAKNSLYLQMNSLRAEDTAVYYCAR

SEQ ID NO: 58 - protein, 97 aa, Artificial
  Synthetic - 3G9HV3 heavy chain sequence for hu3G9
EVQLVESGGGLVQPGGSLRLSCAASGFTFSRYVMSWVRQAPGKGLEWVASISSGGRMYYPDTVKGRFTISRDNAKNSLYLQMNSLRAEDTAVYYCAR

SEQ ID NO: 59 - protein, 76 aa, Artificial
  Synthetic - VH3-7 heavy chain sequence for hu3G9
EVQLVESGGGLVQPGGSLRLSCAASGFTFSWVRQAPGKGLEWVARFTISRDNAKNSLYLQMNSLRAEDTAVYYCAR

SEQ ID NO: 60 - protein, 98 aa, Artificial
  Synthetic - murine for light chain sequence for hu3G9
QIVLTQSPAIMSASPGEKVTLTCSANSSVSSSYLYWYQQKSGSSPKLWIYSTSNLASGVPVRFSGSGSGTSFSLTISSMEAEDAASYFCHQWSTYPPT

SEQ ID NO: 61 - protein, 98 aa, Artificial
  Synthetic - 3G9LV1 light chain sequence for hu3G9
EIVLTQSPATLSLSPGERATLSCSASSSVSSSYLYWYQQKPGQAPRLWIYSTSNLASGVPVRFSGSGSGTDFTLTISSLEPEDFAVYFCHQWSTYPPT

SEQ ID NO: 62 - protein, 98 aa, Artificial
  Synthetic - 3G9LV2 light chain sequence for hu3G9
EIVLTQSPATLSLSPGERATLSCSASSSVSSSYLYWYQQKPGQAPRLWIYSTSNLASGVPARFSGSGSGTDFTLTISSLEPEDFAVYYCHQWSTYPPT

SEQ ID NO: 63 - protein, 98 aa, Artificial
  Synthetic - 3G9LV3 light chain sequence for hu3G9
EIVLTQSPATLSLSPGERATLSCSASSSVSSSYLYWYQQKPGQAPRLWIYSTSNLASGIPARFSGSGSGTDFTLTISSLEPEDFAVYYCHQWSTYPPT

SEQ ID NO: 64 - protein, 98 aa, Artificial
  Synthetic - 3G9LV4 light chain sequence for hu3G9
QIVLTQSPATLSLSPGERATLSCSASSSVSSSYLYWYQQKPGQAPRLWIYSTSNLASGIPARFSGSGSGTDFTLTISSLEPEDFAVYYCHQWSTYPPT

SEQ ID NO: 65 - protein, 98 aa, Artificial
  Synthetic - 3G9LV5 light chain sequence for hu3G9
EIVLTQSPATLSLSPGERATLSCSASSSVSSSYLYWYQQKPGQAPRLLIYSTSNLASGIPARFSGSGSGTDFTLTISSLEPEDFAVYYCHQWSTYPPT

SEQ ID NO: 66 - protein, 71 aa, Artificial
  Synthetic - L6 light chain sequence for hu3G9
EIVLTQSPATLSLSPGERATLSCSWYQQKPGQAPRLLIYGIPARFSGSGSGTDFTLTISSLEPEDFAVYYC

SEQ ID NO: 67 - protein, 98 aa, Artificial
  Synthetic - murine heavy chain sequence for hu8G6
QVQLQQSGPELVRPGVSVKISCKGSSYTFTDYAMHWVKLSHAKSLEWIGVISTYYGNTNYNQKFKGKATMTVDKSSSTAYMELARLTSEDSAVYYCAR

SEQ ID NO: 68 - protein, 98 aa, Artificial
  Synthetic - 8G6HV1 heavy chain sequence for hu8G6
QVQLVQSGAEVKKPGASVKVSCKGSSYTFTDYAMHWVRLAPGQGLEWIGVISTYYGNTNYNQKFKGRATMTVDKSISTAYMELSRLRSDDTAVYYCAR

SEQ ID NO: 69 - protein, 98 aa, Artificial
  Synthetic - 8G6HV2 heavy chain sequences for hu8G6
QVQLVQSGAEVKKPGASVKVSCKASGYTFTDYAMHWVRQAPGQGLEWIGVISTYYGNTNYNQKFKGRATMTVDKSISTAYMELSRLRSDDTAVYYCAR

SEQ ID NO: 70 - protein, 98 aa, Artificial
  Synthetic - 8G6VH3 heavy chain sequence for hu8G6
QVQLVQSGAEVKKPGASVKVSCKASGYTFTDYAMHWVRQAPGQGLEWMGVISTYYGNTNYNQKFKGRATMTVDKSISTAYMELSRLRSDDTAVYYCAR

SEQ ID NO: 71 - protein, 76 aa, Artificial
  Synthetic - VH1-2 heavy chain sequence for hu8G6
QVQLVQSGAEVKKPGASVKVSCKASGYTFTWVRQAPGQGLEWMGRVTMTRDTSISTAYMELSRLRSDDTAVYYCAR

SEQ ID NO: 72 - protein, 99 aa, Artificial
  Synthetic - murine light chain sequence for hu8G6
DIVLTQSPASLAVSLGQRAIISCRASQSVSTSSYSYMYWYQQKPGQSPKFLIKYASNLESGVPARFSGSGSGTDFTLNIHPVEEEDTATYYCQHNWEIP

SEQ ID NO: 73 - protein, 99 aa, Artificial
  Synthetic - 8G6LV1 light chain sequence for hu8G6
DIVLTQSPATLSLSPGERATLSCRASQSVSTSSYSYMYWYQQKPGQAPRFLIKYASNLESGIPARFSGSGSGTDFTLTISSLEPEDFAVYYCQHNWEIP

SEQ ID NO: 74 - protein, 99 aa, Artificial
  Synthetic - 8G6LV2 light chain sequence for hu8G6
EIVLTQSPATLSLSPGERATLSCRASQSVSTSSYSYMYWYQQKPGQAPRFLIKYASNLESGIPARFSGSGSGTDFTLTISSLEPEDFAVYYCQHNWEIP

SEQ ID NO: 75 - protein, 99 aa, Artificial
  Synthetic - 8G6LV3 light chain sequence for hu8G6
EIVLTQSPATLSLSPGERATLSCRASQSVSTSSYSYMYWYQQKPGQAPRLLIKYASNLESGIPARFSGSGSGTDFTLTISSLEPEDFAVYYCQHNWEIP

SEQ ID NO: 76 - protein, 70 aa, Artificial
  Synthetic - L6 light chain sequence for hu8G6
EIVLTQSPATLSLSPGERATLSCWYQQKPGQAPRLLIYGIPARFSGSGSGTDFTLTISSLEPEDFAVYYC

SEQ ID NO: 77 - protein, 237 aa, Artificial
  Synthetic - pKJS195 vector - 3G9 version 5 light chain
MDFQVQIFSFLLISVSVIMSRGEIVLTQSPATLSLSPGERATLSCSASSSVSSSYLYWYQQKPGQAPRLLIYSTSNLASGIPARFSGSGSGTDFTLTISSLEPEDFAVYYCHQWSTYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 468 aa, Artificial
  Synthetic -  pKJS189 vector - 3G9 vector 3 heavy chain
MDFGLSWVFLVLVLKGVQCEVQLVESGGGLVQPGGSLRLSCAASGFTFSRYVMSWVRQAPGKGLEWVASISSGGRMYYPDTVKGRFTISRDNAKNSLYLQMNSLRAEDTAVYYCARGSIYDGYYVFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 79 - protein, 468 aa, Artificial
  Synthetic - pKJS196 vector - aglycosyl-3G9 version 3 heavy chain
MDFGLSWVFLVLVLKGVQCEVQLVESGGGLVQPGGSLRLSCAASGFTFSRYVMSWVRQAPGKGLEWVASISSGGRMYYPDTVKGRFTISRDNAKNSLYLQMNSLRAEDTAVYYCARGSIYDGYYVFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 108 aa, Artificial
  Synthetic -  hu3G9 version 1 light chain
EIVLTQSPATLSLSPGERATLSCSASSSVSSSYLYWYQQKPGQAPRLWIYSTSNLASGVPVRFSGSGSGTDFTLTISSLEPEDFAVYFCHQWSTYPPTFGGGTKVEIK

SEQ ID NO: 81 - protein, 108 aa, Artificial
  Synthetic - hu3G9 version 2 light chain
EIVLTQSPATLSLSPGERATLSCSASSSVSSSYLYWYQQKPGQAPRLWIYSTSNLASGVPARFSGSGSGTDFTLTISSLEPEDFAVYYCHQWSTYPPTFGGGTKVEIK

SEQ ID NO: 82 - protein, 108 aa, Artificial
  Synthetic - hu3G9 version 3 light chain
EIVLTQSPATLSLSPGERATLSCSASSSVSSSYLYWYQQKPGQAPRLWIYSTSNLASGIPARFSGSGSGTDFTLTISSLEPEDFAVYYCHQWSTYPPTFGGGTKVEIK

SEQ ID NO: 83 - protein, 108 aa, Artificial
  Synthetic - hu3G9 version 4 light chain
QIVLTQSPATLSLSPGERATLSCSASSSVSSSYLYWYQQKPGQAPRLWIYSTSNLASGIPARFSGSGSGTDFTLTISSLEPEDFAVYYCHQWSTYPPTFGGGTKVEIK

SEQ ID NO: 84 - protein, 108 aa, Artificial; identical to SEQ ID NO: 2
  Synthetic -  hu3G9 version 5 light chain

SEQ ID NO: 85 - protein, 120 aa, Artificial
  Synthetic - hu3G9 version 1 heavy chain
EVMLVESGGGLVQPGGSLRLSCAASGFTFSRYVMSWVRQAPGKGLEWVASISSGGRMYYPDTVKGRFTISRDSAKNSLYLQMNSLRAEDTAVYYCARGSIYDGYYVFPYWGQGTLVTVSS

SEQ ID NO: 86 - protein, 120 aa, Artificial
  Synthetic - hu3G9 version 2 heavy chain
EVQLVESGGGLVQPGGSLRLSCAASGFTFSRYVMSWVRQAPGKGLEWVASISSGGRMYYPDTVKGRFTISRDSAKNSLYLQMNSLRAEDTAVYYCARGSIYDGYYVFPYWGQGTLVTVSS

SEQ ID NO: 87 - protein, 120 aa, Artificial; identical to SEQ ID NO: 1
  Synthetic - hu3G9 versions 3 and 5 heavy chain

SEQ ID NO: 88 - protein, 11 aa, Artificial
  Synthetic - human FR4 derived from a consensus framework sequence
WGQGTLVTVSS

SEQ ID NO: 89 - protein, 10 aa, Artificial
  Synthetic - human FR4 derived from a consensus framework sequence
FGGGTKVEIK

SEQ ID NO: 90 - protein, 126 aa, Artificial
  Synthetic - hu8G6 version 1 heavy chain
QVQLVQSGAEVKKPGASVKVSCKGSSYTFTDYAMHWVRLAPGQGLEWIGVISTYYGNTNYNQKFKGRATMTVDKSISTAYMELSRLRSDDTAVYYCARGGLRRGDRPSLQYAMDYWGQGTLVTVSS

SEQ ID NO: 91 - protein, 126 aa, Artificial
  Synthetic - hu8G6 version 2 heavy chain
QVQLVQSGAEVKKPGASVKVSCKASGYTFTDYAMHWVRQAPGQGLEWIGVISTYYGNTNYNQKFKGRATMTVDKSISTAYMELSRLRSDDTAVYYCARGGLRRGDRPSLQYAMDYWGQGTLVTVSS

SEQ ID NO: 92 - protein, 126 aa, Artificial
  Synthetic - hu8G6 version 3 heavy chain
QVQLVQSGAEVKKPGASVKVSCKASGYTFTDYAMHWVRQAPGQGLEWMGVISTYYGNTNYNQKFKGRATMTVDKSISTAYMELSRLRSDDTAVYYCARGGLRRGDRPSLQYAMDYWGQGTLVTVSS

(19) United States
(12) Patent Application Publication
(10) Pub. No.: US 2012/0027754 A1
(43) Pub. Date: Feb. 2, 2012

(54) Treatment and Prevention of Chronic Asthma Using Antagonists of Integrin AlphavBeta6

(75) Inventors: Dean Sheppard, Oakland, CA (US); Xiaozhu Huang, Millbrae, CA (US); Shelia M. Violette, Lexington, MA (US)

(73) Assignees: Biogen Idec MA Inc., Cambridge, MA (US); The Regents of the University of California, a California corporation, Oakland, CA (US)

(21) Appl. No.: 13/204,382

(22) Filed: Aug. 5, 2011

Related U.S. Application Data
(63) Continuation of application No. 11/872,876, filed on Oct. 16, 2007.
Application No. 11/872,876 is a continuation-in-part of application No. 13/109,168, filed on May 17, 2011.
Application No. 13/109,168 is a continuation of application No. 11/483,190, filed on Jul. 10, 2006, now Pat. No. 7,943,742.
Application No. 11/483,190 is a continuation-in-part of application No. 12/260,510, filed on Oct. 29, 2008.
Application No. 12/260,510 is a division of application No. 10/507,662, filed on May 20, 2005, now Pat. No. 7,465,449.
(60) Provisional application No. 60/852,638, filed on Oct. 19, 2006.
(60) Provisional application No. 60/773,310, filed on Feb. 15, 2006.
(60) Provisional application No. 60/697,442, filed on Jul. 8, 2005.
(60) Provisional application No. 60/426,286, filed on Nov. 13, 2002.
(60) Provisional application No. 60/364,991, filed on Mar. 13, 2002.

(51) Int. Cl. A61K 39/395; A61P 11/00; A61P 11/06

(52) U.S. Cl. 424/133.1; 424/152.1

(57) ABSTRACT

The present invention relates to methods of asthma treatment and prevention using αᵥβ₆ antagonists, such as αᵥβ₆-binding antibodies. In particular, the invention relates to the discovery of a correlation between reduced expression of αᵥβ₆ and the protection from the increase in airway sensitivity seen in chronic allergen-challenged mice. This protection is associated with protection from the usual allergen-induced increase in airway epithelial mast cells.

The present application claims benefit of priority of U.S. Provisional application Ser. No. 60/852,638, which was filed on Oct. 19, 2006. The entire text of the aforementioned application is incorporated herein by reference in its entirety.

### BACKGROUND OF THE INVENTION

1. Field of the Invention

The present invention relates to methods of asthma treatment and prevention using αᵥβ₆ antagonists, such as αᵥβ₆-binding antibodies. In particular, the invention relates to the discovery of a correlation between reduced expression of αᵥβ₆ and the protection from the increase in airway sensitivity seen in chronic allergen-challenged mice. This protection is associated with protection from the usual allergen-induced increase in airway epithelial mast cells.

2. Related Art

Integrins are cell surface glycoprotein receptors which bind extracellular matrix proteins and mediate cell-cell and cell-extracellular matrix interactions (generally referred to as cell adhesion events) (Ruoslahti, E., J. Clin. Invest. 87:1-5 (1991); Hynes, R. O., Cell 69:11-25 (1992)). These receptors are composed of noncovalently associated alpha (α) and beta (β) chains which combine to give a variety of heterodimeric proteins with distinct cellular and adhesive specificities (Albeda, S. M., Lab. Invest. 68:4-14 (1993)). Recent studies have implicated certain integrins in the regulation of a variety of cellular processes including cellular adhesion, migration, invasion, differentiation, proliferation, apoptosis and gene expression (Albeda, S. M., Lab. Invest. 68:4-14 (1993); Juliano, R., Cancer Met. Rev. 13:25-30 (1994); Ruoslahti, E. and Reed, J. C., Cell 77:477-478 (1994); and Ruoslahti, E. and Giancotti, F. G., Cancer Cells 1:119-126 (1989); Plow, Haas et al. 2000; van der Flier and Sonnenberg 2001).

The αᵥβ₆ receptor is one member of a family of integrins that are expressed as cell surface heterodimeric proteins (Busk, M. et al., J. Biol. Chem. 267(9):5790-5796 (1992)). While the αᵥ subunit can form a heterodimer with a variety of β subunits (β₁, β₃, β₅, β₆ and β₈), the β₆ subunit can only be expressed as a heterodimer with the αᵥ subunit. The αᵥβ₆ integrin is known to be a fibronectin-, latency associated peptide (LAP)- and tenascin C-binding cell surface receptor, interacting with the extracellular matrix through the RGD tripeptide binding sites thereon (Busk, M. et al., J. Biol. Chem. 267:5790-5796 (1992); Weinacker, A. et al., J. Biol. Chem. 269:6940-6948 (1994); Prieto, A. L. et al., Proc. Natl. Acad. Sci. USA 90:10154-10158 (1993)). Although the αᵥβ₆ integrin was first identified and sequenced more than 10 years ago, the biological significance of αᵥβ₆, especially in disease, is still under investigation. The expression of αᵥβ₆ is restricted to epithelial cells where it is expressed at relatively low levels in healthy tissue and significantly upregulated during development, injury, and wound healing (Breuss, J. M. et al., J. Histochem. Cytochem. 41:1521-1527 (1993); Breuss, J. M. et al., J. Cell Sci. 108:2241-2251 (1995); Koivisto, L. et al., Cell Adhes. Communic. 7:245-257 (1999); Zambruno, G. et al., J. Cell Biol. 129(3):853-865 (1995); Hakkinen, L. et al., J. Histochem. Cytochem. 48(6):985-998 (2000)).

The αᵥβ₆ integrin may have multiple regulatory functions in airway remodeling. Recent studies have shown that increased airway epithelial expression of the αᵥβ₆ integrin may contribute to the increased activation of latent TGF-β. Previous studies have demonstrated that the integrin αᵥβ₆ binds and activates latent TGF-β1 and that αᵥβ₆ has a very restricted pattern of tissue expression being only expressed in epithelium, particularly lung and skin epithelium (Munger, J. S., et al., Cell 96: 319-328 (1999); Huang, X. Z., et al., J. Cell Biol. 133: 921-928; Breuss, J. M., et al., J. Cell Sci. 108: 2241-2251)). The cytoplasmic domain of the β₆ subunit contains a unique 11-amino acid sequence that is important in mediating αᵥβ₆ regulated cell proliferation, MMP production, migration, and pro-survival (Li, X. et al., J. Biol. Chem. 278(43):41646-41653 (2003); Thomas, G. J. et al., J. Invest. Derm. 117(1):67-73 (2001); Thomas, G. J. et al., Br. J. Cancer 87(8):859-867 (2002); Janes, S. M. and Watt, F. M., J. Cell Biol 166(3):419-431 (2004)). The β₆ subunit has been cloned, expressed and purified (Sheppard et al., U.S. Pat. No. 6,787,322 B2, the disclosure of which is incorporated herein by reference in its entirety), and function-blocking antibodies that selectively bind to the αᵥβ₆ integrin have been reported (Weinreb et al., J. Biol. Chem. 279:17875-17877 (2004), the disclosure of which is incorporated herein by reference in its entirety). Antagonists of αᵥβ₆ (including certain monoclonal antibodies) have also been suggested as possible treatments for certain forms of acute lung injury and fibrosis (see U.S. Pat. No. 6,692,741 B2 and WO 99/07405, the disclosures of which are incorporated herein by reference in their entireties).

αᵥβ₆ can bind to several ligands including fibronectin, tenascin, and the latency associated peptide-1 and -3 (LAP1 and LAP3), the N-terminal 278 amino acid region of the latent precursor form of TGF-β1 and TGF-β2, respectively, through a direct interaction with an arginine-glycine-aspartate (“RGD”) motif (Busk, M. et al., J. Biol. Chem. 267(9):5790-5796 (1992); Yokosaki, Y. et al., J. Biol. Chem. 271(39):24144-24150 (1996); Huang, X. Z. et al., J. Cell. Sci. 111:2189-2195 (1998); Munger, J. S. et al., Cell 96:319-328 (1999)). The TGF-β cytokine is synthesized as a latent complex which has the N-terminal LAP non-covalently associated with the mature active C-terminal TGF-β cytokine. The latent TGF-β complex cannot bind to its cognate receptor and thus is not biologically active until converted to an active form (Barcellos-Hoff, M. H., J. Mamm. Gland Biol. 1(4):353-363 (1996); Gleizes, P. E. et al., Stem Cells 15(3):190-197 (1997); Munger, J. S. et al., Kid. Int. 51:1376-1382 (1997); Khalil, N., Microbes Infect. 1(15):1255-1263 (1999)). αᵥβ₆ binding to LAP1 or LAP3 leads to activation of the latent precursor form of TGF-β1 and TGF-β3 (Munger, J. S. et al., Cell 96:319-328 (1999)), proposed as a result of a conformational change in the latent complex allowing TGF-β to bind to its receptor. Thus, upregulated expression of αᵥβ₆ can lead to local activation of TGF-β which in turn can activate a cascade of events downstream events.

The TGF-β1 cytokine is a pleiotropic growth factor that regulates cell proliferation, differentiation, and immune responses (Wahl, S. M., J. Exp. Med. 180:1587-1590 (1994); Massague, J., Annu. Rev. Biochem. 67:753-791 (1998); Chen, W. and Wahl, S. M., TGF-β: Receptors, Signaling Pathways and Autoimmunity, Basel: Karger, pp. 62-91 (2002); Thomas, D. A. and Massague, J., Cancer Cell 8:369-380 (2005); Li et al., Annul. Rev. Immunol. 24: 99-146 (2006). TGF-β expression in the remodeled airway of WT mice could account for many of the features of airway remodeling including higher numbers of macrophages and mast cells. Because TGF-β stimulates fibroblasts to produce ECM proteins such as collagen (Blobe, G. C., et al., New Engl. J. Med. 342: 1350-1358 (2000)), the increased levels of TGF-β expression detected in wild-type mice challenged with OVA could contribute to fibroblast collagen synthesis, whereas the reduction in TGF-β expression could account for the reduced collagen synthesis noted in OVA-challenged mice (Cho, J. Y., et al., J. Clin. Invest. 113: 551-560 (2004). The epithelial expression of TGF-β1 mRNA and protein correlates with the number of intraepithelial macrophages whereas intraepithelial mast cell numbers correlate with epithelial TGF-β1 mRNA expression, suggesting a role for TGF-β1 in recruiting macrophages into allergen challenged airway epithelium (Boer, W. I., et al., Am. J. Respir. Crit. Care Med. 158: 1951-1957 (1998).

The generation of potent and selective anti-αᵥβ₆ monoclonal antibodies (mAbs) that bind to both the human and murine forms of αᵥβ₆ and block the binding of αᵥβ₆ to its ligands and αᵥβ₆ mediated activation of TGF-β1 has been previously described (Weinreb, P. H. et al., J. Biol. Chem. 279(17):17875-17887 (2004); see also U.S. patent application Ser. No. 11/483,190 by Violette et al., entitled “Humanized αᵥβ₆ Antibodies and Uses Thereof,” filed on Jul. 10, 2006, which is incorporated herein by reference in its entirety). As also described in PCT Publication WO 03/100033, herein incorporated in its entirety by reference, high affinity antibodies against αᵥβ₆, including the identification and analysis of key amino acid residues in the complementary determining regions (CDRs) of such antibodies, were discovered and characterized. In particular, these high affinity antibodies (a) specifically bind to αᵥβ₆; (b) inhibit the binding of αᵥβ₆ to its ligand such as LAP, fibronectin and tenascin with an IC₅₀ value lower than that of 10D5 (International Patent Application Publication WO 99/07405); (c) block activation of TGF-β; (d) contain certain amino acid sequences in the CDRs that provide binding specificity to αᵥβ₆; (e) specifically bind to the β₆ subunit; and/or (f) recognize αᵥβ₆ in immunostaining procedures, such as immunostaining of paraffin-embedded tissues.

WO 03/100033 also describes the discovery that antibodies that bind to αᵥβ₆ can be grouped into biophysically distinct classes and subclasses. One class of antibodies exhibits the ability to block binding of a ligand (e.g., LAP) to αᵥβ₆ (blockers). This class of antibodies can be further divided into subclasses of cation-dependent blockers and cation-independent blockers. Some of the cation-dependent blockers contain an arginine-glycine-aspartate (RGD) peptide sequence, whereas the cation-independent blockers do not contain an RGD sequence. Another class of antibodies exhibits the ability to bind to αᵥβ₆ and yet does not block binding of αᵥβ₆ to a ligand (nonblockers).

Furthermore, WO 03/100033 discloses antibodies comprising heavy chains and light chains whose complementarity determining regions (CDR) 1, 2 and 3 consist of certain amino acid sequences that provide binding specificity to αᵥβ₆. WO 03/100033 also provides for antibodies that specifically bind to αᵥβ₆ but do not inhibit the binding of αᵥβ₆ to latency associated peptide (LAP) as well as antibodies that bind to the same epitope.

WO 03/100033 further discloses cells of hybridomas 6.1A8, 6.2B10, 6.3G9, 6.8G6, 6.2B1, 6.2A1, 6.2E5, 7.1G10, 7.7G5, and 7.1C5, isolated nucleic acids comprising a coding sequences and isolated polypeptides comprising amino acid sequences of the anti-αᵥβ₆ antibodies. In particular, WO 03/100033 discloses anti-αᵥβ₆ antibodies comprising heavy and light chain polypeptide sequences as antibodies produced by hybridomas 6.1A8, 6.3G9, 6.8G6, 6.2B1, 6.2B10, 6.2A1, 6.2E5, 7.1G10, 7.7G5, or 7.1C5. Several of the hybridomas were deposited at the American Type Culture Collection (“ATCC”; P.O. Box 1549, Manassas, Va. 20108, USA) under the Budapest Treaty. In particular, hybridoma clones 6.3G9 and 6.8G6 were deposited on Aug. 16, 2001, and have accession numbers ATCC PTA-3649 and PTA-3645, respectively. The murine antibodies produced by hybridomas 6.3G9 and 6.8G6 are being further explored in the present application for their potential development as humanized antibodies.

The murine monoclonal antibody 3G9 is a murine IgG1, kappa antibody isolated from the β₆ integrin −/− mouse (Huang et al., J. Cell Biol. 133:921-928 (1996)) immunized with human soluble αᵥβ₆. The 3G9 antibody specifically recognizes the αᵥβ₆ integrin epitope which is expressed at upregulated levels during injury, fibrosis and cancer (see, e.g., Thomas et al., J. Invest. Dermatology 117:67-73 (2001); Brunton et al., Neoplasia 3: 215-226 (2001); Agrez et al., Int. J. Cancer 81:90-97 (1999); Breuss, J. Cell Science 108:2241-2251 (1995)). It does not bind to other αᵥ integrins and is cross-reactive to both human and murine molecules. The murine monoclonal antibody 3G9 has been described to block the binding of αᵥβ₆ to LAP as determined by blocking of ligand binding either to purified human soluble αᵥβ₆ or to β₆-expressing cells, thereby inhibiting the pro-fibrotic activity of TGF-β receptor activation (see WO 03/100033). It has also been shown to inhibit αᵥβ₆-mediated activation of TGF-β with an IC₅₀ value lower than one of the known αᵥβ₆ antibodies, 10D5 (Huang et al., J. Cell Sci. 111:2189-2195 (1998)).

The murine monoclonal antibody 8G6 is a murine IgG1, kappa antibody which also recognizes the αᵥβ₆ integrin epitope, as described in WO 03/100033. The murine monoclonal antibody 8G6 is a cation-dependent, high affinity blocker of αᵥβ₆ displaying the ability to inhibit αᵥβ₆-mediated activation of TGF-β with an IC₅₀ value lower than 10D5 (see WO 03/100033).

Both the 3G9 and 8G6 murine antibodies were effective in preventing fibrosis of the kidney and lung, as described in WO 03/100033. Furthermore, the murine antibody 3G9 was able to effectively inhibit tumor growth in a human tumor xenograft model, suggesting the potential role of αᵥβ₆ in cancer pathology and the effectiveness of such blockade using antibodies directed at αᵥβ₆.

Asthma is a serious chronic condition affecting an estimated 10 million Americans. Asthma is characterized by (i) bronchoconstriction, (ii) excessive mucus production, and (iii) inflammation and swelling of airways. These conditions cause widespread and variable airflow obstruction thereby making it difficult for the asthma sufferer to breathe. Asthma further includes acute episodes or attacks of additional airway narrowing via contraction of hyper-responsive airway smooth muscle. Other obstructive diseases such as COPD may also have a reversible component caused by one or more of the above mentioned three elements.

Asthma generally includes excessive mucus production in the bronchial tree. Usually, there is a general increase in bulk (hypertrophy) of the large bronchi and chronic inflammatory changes in the small airways. Excessive amounts of mucus are found in the airways and semisolid plugs of mucus may occlude some small bronchi. Also, the small airways are narrowed and show inflammatory changes. The reversible aspects of COPD include partial airway occlusion by excess secretions, and airway narrowing secondary to smooth muscle contraction, bronchial wall edema and inflammation of the airways.

In asthma, chronic inflammatory processes in the airway play a central role in increasing the resistance to airflow within the lungs. Many cells and cellular elements are involved in the inflammatory process, particularly mast cells, eosinophils T lymphocytes, neutrophils, epithelial cells, and even airway smooth muscle itself. The reactions of these cells result in an associated increase in the existing sensitivity and hyper-responsiveness of the airway smooth muscle cells that line the airways to the particular stimuli involved.

The chronic nature of asthma can also lead to remodeling of the airway wall (i.e., structural changes such as thickening or edema) which can further affect the function of the airway wall and influence airway hyper-responsiveness. Other physiologic changes associated with asthma include excess mucus production, and if the asthma is severe, mucus plugging, as well as ongoing epithelial denudation and repair. Epithelial denudation exposes the underlying tissue to substances that would not normally come in contact with them, further reinforcing the cycle of cellular damage and inflammatory response.

In susceptible individuals, asthma symptoms include recurrent episodes of shortness of breath (dyspnea), wheezing, chest tightness, and cough. Currently, asthma is managed by a combination of stimulus avoidance and pharmacology.

Stimulus avoidance is accomplished via systematic identification and minimization of contact with each type of stimuli. It may, however, be impractical and not always helpful to avoid all potential stimuli.

Asthma is managed pharmacologically by: (1) long term control through use of anti-inflammatories and long-acting bronchodilators and (2) short term management of acute exacerbations through use of short-acting bronchodilators. Both of these approaches require repeated and regular use of the prescribed drugs. High doses of corticosteroid anti-inflammatory drugs can have serious side effects that require careful management. In addition, some patients are resistant to steroid treatment. The difficulty involved in patient compliance with pharmacologic management and the difficulty of avoiding stimulus that triggers asthma are common barriers to successful asthma management. Thus, current management techniques are neither completely successful nor free from side effects.

### BRIEF SUMMARY OF THE INVENTION

The present invention relates to methods of asthma treatment and prevention using αᵥβ₆-binding antagonists, such as αᵥβ₆-binding antibodies. In particular, the invention relates to the discovery of a correlation between reduced expression of αᵥβ₆ and the protection from the increase in airway sensitivity seen in chronic allergen-challenged mice. This protection is associated with protection from the usual allergen-induced increase in airway epithelial mast cells.

More particularly, the present invention provides methods of treating a mammal having, or at risk of having, one or more symptoms of asthma or an asthma-related condition. In one embodiment, the method comprises administering to the mammal a therapeutically effective dose of a ligand that recognizes and/or binds to the integrin αᵥβ₆. In some embodiments, the ligand is an antagonist of one or more subunits of integrin αᵥβ₆.

In certain such embodiments, the antagonist is an antibody or a fragment thereof that binds to one or more subunits of the integrin αᵥβ₆, i.e., to the αᵥ and/or β6 subunits. In some embodiments, the antibody is administered to a patient at risk of having symptoms of asthma or asthma-related symptoms. In some embodiments, the antibody is administered parenterally. In other embodiments, the antibody is administered as an aerosol. In some embodiments, the antibody is administered intranasally.

In some embodiments, the antibody is a monoclonal antibody, a chimeric, primatized or humanized monoclonal antibody. In certain such embodiments, the monoclonal antibody is selected from the group consisting of 2A1, 2E5, 1A8, 2B10, 2B1, 1G10, 7G5, 1C5, 8G6, 3G9, 10D5 and CSβ6, and more particularly 3G9 or 8G6. In other embodiments, the monoclonal antibody is a humanized antibody, such as hu3G9 (BG00011) or hu8G6.

Suitable embodiments according to this aspect of the invention use αvβ6 integrin-binding ligands which are αvβ6-binding antibodies or αvβ6 epitope-binding fragments thereof. According to certain such embodiments, the antibodies are monoclonal antibodies (which may be chimeric, primatized or humanized), including those disclosed in U.S. patent application publication no. US 2005/0255102 A1, the disclosure of which is incorporated herein in its entirety. Suitable such antibodies include, but are not limited to, the αvβ6-binding monoclonal antibodies designated 1A8, 3G9, 8G6, 2B1, 2B10, 2A1, 2E5, IG10, 7G5, 1C5, 10D5 (ATCC deposit no. HB12382) and CSβ6, as well as fragments, chimeras and hybrids thereof. Particularly suitable for use in such embodiments of the invention are monoclonal antibodies 3G9 and 8G6. Also particularly suitable for use in such embodiments of the invention are humanized monoclonal antibodies, such as the humanized 3G9 antibody designated hu3G9 (BGOOOI 1) and the humanized 8G6 antibody designated hu8G6, which are discussed herein above and described in further detail in PCT publication No. WO2007/008712 and its counterpart U.S. Application, Serial No. U.S. Ser. No. 11/483,190, each of which is incorporated herein by reference in its entirety.

In additional therapeutic embodiments of the invention, the αvβ6-binding ligands (e.g., αvβ6-binding antibodies) are administered to a patient in conjunction with one or more such therapeutic agents for the treatment of asthma.

In certain aspects, the invention relates to methods of treating a mammal having or at risk of having one or more symptoms of asthma or an asthma related condition, comprising administering to the mammal a therapeutically effective dose of a ligand to the integrin αvβ6. In certain embodiments, the ligand is an antagonist of one or more subunits of integrin αvβ6.

In particular embodiments, the invention relates to methods of treating a mammal having or at risk of having one or more symptoms of asthma or an asthma related condition, comprising administering to the mammal a therapeutically effective dose of an antibody or a fragment thereof that binds to one or more the subunits of the integrin αvβ6. The antibody may be administered through any route traditionally employed for administration of a pharmaceutical agent, particularly and protein-based pharmaceutical agent, and may include parenteral, oral, aerosol, or intranasal administration.

Preferably the antibody being administered is a monoclonal antibody. For example, the monoclonal antibody is a chimeric, primatized or humanized monoclonal antibody.

In specific embodiments, the methods of the invention employ a monoclonal antibody is selected from the group consisting of 2A1, 2E5, 1A8, 2B10, 2B1, 1G10, 7G5, 1C5, 8G6, 3G9, 10D5 and CSβ6. In particular embodiments, the humanized monoclonal antibody is hu3G9 (BG00011).

In specific embodiments, the therapeutic methods of the invention comprise administering an antibody that comprises heavy and light chain variable domains of SEQ ID NO:1 and SEQ ID NO:2, respectively.

In other specific embodiments, the therapeutic methods of the invention comprise administering a humanized monoclonal antibody that comprises a heavy chain whose CDR 1, 2 and 3 comprise amino acids 31-35, 50-65 and 98-109 of SEQ ID NO:1, respectively.

In still other embodiments, the therapeutic methods of the invention comprise administering a humanized monoclonal antibody that comprises a light chain whose CDR 1, 2 and 3 comprise amino acids 24-35, 51-57 and 90-98, respectively of SEQ ID NO:2, respectively.

In still other embodiments, the therapeutic methods of the invention comprise administering a humanized monoclonal antibody that comprises a heavy chain whose framework regions (FR) 1, 2, 3 and 4 comprise amino acid residues 1-30, 36-49, 66-97 and 110-120 of SEQ ID NO: 1, respectively.

In still other embodiments, the therapeutic methods of the invention comprise administering a humanized monoclonal antibody that comprises a light chain whose framework regions (FR) 1, 2, 3 and 4 comprise amino acid residues 1-23, 36-50, 58-89 and 99-108, respectively, of SEQ ID NO: 2.

In still other embodiments, the therapeutic methods of the invention comprise administering a humanized monoclonal antibody that comprises one or more of the following amino acid substitutions in the heavy chain consisting of Q3M and N74S of SEQ ID NO: 1 and/or one or more of the following amino acid substitutions in the light chain consisting of E1Q, L47W, I58V, A60V and Y87F of SEQ ID NO: 2.

In still other embodiments, the therapeutic methods of the invention comprise administering a humanized monoclonal antibody that comprises a heavy chain version selected from the group consisting of heavy chain version 1 (“HV1”); heavy chain version 2 (“HV2”), and heavy chain version 3, wherein the HV1 heavy chain consists of amino acid substitutions Q3M and N74S of SEQ ID NO: 1; the HV2 heavy chain consists of amino acid substitution N74S of SEQ ID NO: 1; and the HV3 heavy chain consists of SEQ ID NO: 1.

In further embodiments, the therapeutic methods of the invention comprise administering a humanized monoclonal antibody comprises a light chain version selected from the group consisting of light chain version 1 (“LV1”), light chain version 2 (“LV2”), light chain version 3 (“LV3”), light chain version 4 (“LV4”) and light chain version 5 (“LV5”), wherein LV1 light chain consists of amino acid substitutions L47W, 158 V, A60V and Y87F of SEQ ID NO: 2; the LV2 light chain consists of amino acid substitutions L47W and 158V of SEQ ID NO: 2; the LV3 light chain consists of amino acid substitution L47W of SEQ ID NO: 2; the LV4 light chain consists of amino acid substitutions E1Q and L47W of SEQ ID NO: 2 and the LV5 light chain consists of SEQ ID NO: 2.

In additional embodiments, the therapeutic methods of the invention comprise administering a humanized monoclonal antibody that comprises an aglycosyl light chain whose CDRs are derived from the murine 3G9 antibody.

In still other embodiments, the therapeutic methods of the invention comprise administering a humanized monoclonal antibody that contains a light chain variable domain wherein the CDR1 region contains an asparagine to serine substitution at amino acid 26 of SEQ ID NO:2.

In still other embodiments, the therapeutic methods of the invention comprise administering a humanized monoclonal antibody that contains an asparagine to glutamine substitution in the heavy chain version 3 occurring at amino acid residue 319 of SEQ ID NO:7.

In still other embodiments, the therapeutic methods of the invention comprise administering a humanized monoclonal antibody that comprises the heavy chain version 3 produced by a recombinant vector comprising plasmid pKJS189 (SEQ ID NO:6) and the light chain version 5 produced by a recombinant vector comprising plasmid pKJS195 (SEQ ID NO:5).

In still other embodiments, the therapeutic methods of the invention comprise administering a humanized monoclonal antibody that comprises the aglycosyl heavy chain version 3 produced by a recombinant vector comprising plasmid pKJS196 (SEQ ID NO:7) and the light chain version 5 produced by a recombinant vector comprising plasmid pKJS195 (SEQ ID NO:5).

In still other embodiments, the therapeutic methods of the invention comprise administering a humanized monoclonal antibody that comprises:

a) a heavy chain CDR1 that comprises a sequence selected from the group consisting of any one of SEQ ID NOs 101-105;

b) a heavy chain CDR2 that comprises a sequence selected from the group consisting of any one of SEQ ID NOs 106-111;

c) a heavy chain CDR3 that comprises a sequence selected from the group consisting of any one of SEQ ID NOs 112-117.

In still other embodiments, the therapeutic methods of the invention comprise administering a humanized monoclonal antibody that comprises:

a) a light chain CDR1 that comprises a sequence selected from the group consisting of any one of SEQ ID NOs: 118-123;

b) a light chain CDR2 that comprises a sequence selected from the group consisting of any one of SEQ ID NOs:124-127; and

c) a light chain CDR3 that comprises a sequence selected from the group consisting of any one of SEQ ID NOs 128-133.

In still other embodiments, the therapeutic methods of the invention comprise administering a humanized monoclonal antibody that is defined herein as hu8G6.

In the therapeutic methods of the invention the antibody being administered is one that binds to a β6 subunit of the integrin αvβ6.

In more specific embodiments, the antibody binds β6 subunit of the integrin αvβ6 in the αvβ6 complex but does not bind αv alone.

In various aspects of the present invention the methods employ an antagonist (be it a ligand of the integrin αvβ6 or an antibody such as those exemplified herein) that is conjugated with at least one detectable label selected from the group consisting of a chromogenic label, an enzyme label, a radioisotopic label, a non-radioactive isotopic label, a fluorescent label, a chemiluminescent label, an X-radiographic label, a spin label and a nuclear magnetic resonance contrast agent label.

In specific aspects of the invention, the antagonist is a ligand for αvβ6. In other specific embodiments, the antagonist is an antisense nucleic acid.

In specific embodiments, such and antagonist is conjugated with at least one detectable label, for example, a detectable label is selected from the group consisting of a chromogenic label, an enzyme label, a radioisotopic label, a non-radioactive isotopic label, a fluorescent label, a chemiluminescent label, an X-radiographic label, a spin label and a nuclear magnetic resonance contrast agent label.

Examples of the chromogenic label may include but are not limited to labels selected from the group consisting of diaminobenzidine and 4 hydroxyazo-benzene-2-carboxylic acid.

Examples of enzyme labels may include but are not limited to enzymes selected from the group consisting of malate dehydrogenase, staphylococcal nuclease, delta 5 steroid isomerase, yeast alcohol dehydrogenase, alpha glycerol phosphate dehydrogenase, triose phosphate isomerase, peroxidase, alkaline phosphatase, asparaginase, glucose oxidase, β galactosidase, ribonuclease, urease, catalase, glucose 6 phosphate dehydrogenase, glucoamylase and acetylcholine esterase.

Examples of radioisotopic labels may include but are not limited to radioisotopes selected from the group consisting of 3H, 111In, 125I, 131I, 32P, 35S, 14C, 51Cr, 57To, 58Co, 59Fe, 75Se, 152Eu, 90Y, 67Cu, 217Ci, 211At, 212Pb, 47Sc and 109Pd.

Examples of non-radioisotope labels include but are not limited to non-radioactive isotopic labels selected from the group consisting of 157Gd, 55Mn, 162Dy, 52Tr, 56Fe, 99 mTc and 112In.

Examples of fluorescent label include but are not limited to fluorescent labels selected from the group consisting of a 152Eu label, a fluorescein label, an isothiocyanate label, a rhodamine label, a phycoerythrin label, a phycocyanin label, an allophycocyanin label, a Green Fluorescent Protein (GFP) label, an o phthaldehyde label and a fluorescamine label.

Other aspects of the present invention relate to methods of treating a mammal having or at risk of having one or more symptoms of asthma or an asthma related condition, comprising co-administering to the mammal a therapeutically effective dose of an antibody or a fragment thereof that binds to one or more the subunits of the integrin αvβ6 and one or more additional active agents.

In more specific embodiments, the one or more additional active agents are selected from the group consisting of:

(a) one or more antihistamines; (b) one or more corticosteroids; (c) one or more leukotriene antagonists; (d) one or more decongestants; (e) one or more non-steroidal anti-inflammatory agents; (f) one or more anticholinergic agents; (g) one or more short or long-acting beta-agonists; and (i) one or more methylxanthines.

Another aspect of the present invention relates to methods of alleviating edema in the lung airways of an animal comprising administering to the animal a therapeutically effective dose of an antibody or a fragment thereof that binds to one or more the subunits of the integrin αvβ6. More specifically, the methods relate to alleviating edema that is asthma-associated edema. In specific aspects, the edema is cardiogenic pulmonary edema. In other aspects, the edema is non-cardiogenic edema.

In another aspect of the invention there are provided methods of decreasing mucus production in the lung airways of an animal comprising administering to the animal a therapeutically effective dose of an antibody or a fragment thereof that binds to one or more the subunits of the integrin αvβ6. More specifically, the animal being treated in these methods is suffering from asthma.

Still another aspect of the invention relates to methods of decreasing epithelial denudation of lung tissue in an animal comprising administering to the animal a therapeutically effective dose of an antibody or a fragment thereof that binds to one or more the subunits of the integrin αvβ6.

In still further aspects, the invention relates to methods of alleviating one or more of the symptoms of an asthma-related condition selected from the group consisting of fibrosis of epithelial tissue of the lung, acute lung injury, rhinitis, anaphylaxis, sinusitis, hay fever, vocal cord disfunction and gastroesophageal reflux disease in an animal comprising administering to the animal a therapeutically effective dose of an antibody or a fragment thereof that binds to one or more the subunits of the integrin αvβ6.

In still other aspects, the invention relates to methods of treating COPD in an animal comprising administering to said animal a therapeutically effective dose of an antibody or a fragment thereof that binds to one or more said subunits of the integrin αᵥβ₆.

Other aspects of the present invention relate to methods of treating a mammal having or at risk of having one or more symptoms of asthma or an asthma related condition, comprising co-administering to the mammal a therapeutically effective dose of a therapeutically effective dose of a ligand to the integrin αvβ6 and one or more additional active agents.

In more specific embodiments, the one or more additional active agents are selected from the group consisting of: (a) one or more antihistamines; (b) one or more corticosteroids; (c) one or more leukotriene antagonists; (d) one or more decongestants;

(e) one or more non-steroidal anti-inflammatory agents; (f) one or more anticholinergic agents; (g) one or more short or long-acting beta-agonists; and (i) one or more methylxanthines

Other preferred embodiments of the present invention will be apparent to one of ordinary skill in light of the following drawings and description of the invention, and of the claims.

### BRIEF DESCRIPTION OF THE DRAWINGS

FIG. 1 shows a protocol for inducing a chronic allergic animal model. Intranasal OVA challenges (20 ng/50 μl in saline) were administered on days 26, 29 and 32 under isoflurane anesthesia and then repeated twice a week for 7 weeks. A higher dose ovalbumin (OVA) challenge (1 mg/50 ul in saline) was performed for another 7 weeks. 24 hours after the last challenge, mice were analyzed for lung mechanics and lung inflammation.

FIG. 2 shows lung inflammation in β6 knockout mice challenged with OVA. The total cell numbers were counted in wild-type mice challenged with saline and wild-type mice challenged with OVA. In addition, cell numbers were counted for β6 knockout mice challenged with saline and β6 knockout mice challenged with OVA. Cell numbers were counted for total cells, macrophages, eosinophils, leukocytes and polymorphonuclear leukocytes.

FIG. 3 shows the protected airway responsiveness in β6 knockout mice chronically challenged with OVA. Mice were given increasing doses of acetylcholine (0.03, 0.1, 0.3, 1 and 3 μg/g body weight) administered through the tail vein to generate a concentration-response curve. A concentration-response curve was measured for wild-type mice challenged with saline and wild-type mice challenged with OVA, along with β6 knockout mice challenged with saline and β6 knockout mice challenged with OVA.

FIG. 4 shows the increased sub-epithelial fibrosis of both wild-type and β6 knockout mice chronically OVA challenged. Col (Collagen) volume/μM BM (basement membrane) was measured for both wild-type and β6 knockout mice chronically OVA challenged.

FIG. 5 shows the increased α-SMC actin in both wild-type and β6 knockout mice chronically stimulated with antigen. This figure shows stained cells of both saline- and OVA-challenged wild-type and β6 knockout mice.

FIG. 6 shows the reduced intraepithelial mast cells in β6 knockout mice chronically OVA challenged. The cell number/cm BM (basement membrane) of both the control and OVA challenged wild-type and β6 knockout mice cells were counted.

FIG. 7 shows the pulmonary inflammatory response in mice chronically challenged with antigen. This figure shows stained cells of both saline- and OVA-challenged wild-type and β6 knockout mice.

### DETAILED DESCRIPTION OF THE INVENTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are described hereinafter.

### Definitions

About: As used herein when referring to any numerical value, the term “about” means a value of ±10% of the stated value (e.g., “about 50° C.” encompasses a range of temperatures from 45° C. to 55° C., inclusive; similarly, “about 100 mM” encompasses a range of concentrations from 90 mM to 110 mM, inclusive).

Antagonist: As used herein, the term “antagonist” refers to a compound, molecule, moiety or complex that reduces, substantially reduces or completely inhibits the biological and/or physiological effects of the αᵥβ₆ integrin in a cell, tissue or organism. Antagonists, which may be ligands for αᵥβ₆, may carry out such effects in a variety of ways, including but not limited to competing with another ligand for binding to αᵥβ₆ on the cell surface; interacting with αᵥβ₆ in such a way as to reduce, substantially reduce or inhibit the ability of the integrin to bind other ligands; binding to and inducing a conformational change in cell surface αᵥβ₆ such that the integrin assumes a structure to which other ligands can no longer bind (or can bind only with reduced or substantially reduced affinity and/or efficiency); inducing a physiological change (e.g., increase in intracellular signaling complexes; increase in transcriptional inhibitors; reduction in cell surface αᵥβ₆ expression; etc.) in cells, tissues or organisms such that the binding of other ligands, or the physiological signal induced by such ligands upon binding to the αᵥβ₆ on the cell, is reduced, substantially reduced or completely inhibited; and other mechanisms by which antagonists may carry out their activities, that will be familiar to the ordinarily skilled artisan. As the ordinarily skilled artisan will understand, an antagonist may have a similar structure to another αᵥβ₆-binding moiety (e.g., an αᵥβ₆-binding ligand) that it antagonizes (e.g., the antagonist may be a mutein, variant, fragment or derivative of the agonist), or may have a wholly unrelated structure. In certain aspects of the invention, the antagonist may be any antibody, such as for example, an αᵥβ₆-binding antibody.

Bound: As used herein, the term “bound” refers to binding or attachment that may be covalent, e.g., by chemically coupling, or non-covalent, e.g., ionic interactions, hydrophobic interactions, hydrogen bonds, etc. Covalent bonds can be, for example, ester, ether, phosphoester, thioester, thioether, urethane, amide, amine, peptide, imide, hydrazone, hydrazide, carbon-sulfur bonds, carbon-phosphorus bonds, and the like. The term “bound” is broader than and includes terms such as “coupled,” “conjugated” and “attached.”

Conjugate/conjugation: As used herein, “conjugate” refers to the product of covalent attachment of a moiety, e.g., a chemical or radioisotope, to a ligand that binds to αᵥβ₆, e.g., an αᵥβ₆-binding antibody or fragment thereof “Conjugation” refers to the formation of a conjugate as defined in the previous sentence. Any method normally used by those skilled in the art of conjugation of chemicals or radioisotopes to biologically active materials, such as proteins or polypeptides (including antibodies) can be used in the present invention.

Disease, disorder, condition: As used herein, the terms “disease” or “disorder” refer to any adverse condition of a human or animal including tumors, cancer, allergies, addiction, autoimmunity, infection, poisoning or impairment of optimal mental or bodily function. “Conditions” as used herein includes diseases and disorders but also refers to physiologic states. For example, fertility is a physiologic state but not a disease or disorder. Compositions of the invention suitable for preventing pregnancy by decreasing fertility would therefore be described as a treatment of a condition (fertility), but not a treatment of a disorder or disease. Other conditions are understood by those of ordinary skill in the art.

Effective Amount: As used herein, the term “effective amount” refers to an amount of a given compound, conjugate or composition that is necessary or sufficient to realize a desired biologic effect. An effective amount of a given compound, conjugate or composition in accordance with the methods of the present invention would be the amount that achieves this selected result, and such an amount can be determined as a matter of routine by a person skilled in the art, using assays that are known in the art and/or that are described herein, without the need for undue experimentation. For example, an effective amount for treating or preventing cancer metastasis could be that amount necessary to prevent migration and invasion of a tumor cell across the basement membrane or across an endothelial layer in vivo. The term is also synonymous with “sufficient amount.” The effective amount for any particular application can vary depending on such factors as the disease, disorder or condition being treated, the particular composition being administered, the route of administration, the size of the subject, and/or the severity of the disease or condition. One of ordinary skill in the art can determine empirically the effective amount of a particular compound, conjugate or composition of the present invention, in accordance with the guidance provided herein, without necessitating undue experimentation.

One, a, or an: When the terms “one,” “a,” or “an” are used in this disclosure, they mean “at least one” or “one or more,” unless otherwise indicated. As such, the terms “a” (or “an”), “one or more,” and “at least one” can be used interchangeably herein.

Peptide, polypeptide, protein: As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. In accordance with this definition, polypeptides used in the present invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue. Preferred polypeptides used in accordance with the invention include polypeptides that are ligands or that bind to an αᵥβ₆ integrin on the surface of a cell, including but not limited to antibodies (especially monoclonal antibodies) that recognize and bind to one or more epitopes on αᵥβ₆.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

Also included as polypeptides of the present invention are fragments, derivatives, analogs, or variants of the foregoing polypeptides, and any combination thereof. The terms “fragment,” “variant,” “derivative” and “analog” when referring to anti-αᵥβ₆ antibodies or antibody polypeptides include any polypeptides which retain at least some of the antigen-binding properties of the corresponding native antibody or polypeptide, i.e., those polypeptides that retain the ability to bind to one or more epitopes on an αᵥβ₆ integrin. Fragments of polypeptides of the present invention include proteolytic fragments, as well as deletion fragments, in addition to specific antibody fragments discussed elsewhere herein. Variants of anti-αᵥβ₆ antibodies and antibody polypeptides useful in accordance with the present invention include fragments as described above, and also polypeptides with altered amino acid sequences due to amino acid substitutions, deletions, or insertions. Variants may occur naturally or be non-naturally occurring. Non-naturally occurring variants may be produced using art-known mutagenesis techniques. Variant polypeptides may comprise conservative or non-conservative amino acid substitutions, deletions or additions. Derivatives of anti-αᵥβ₆ antibodies and antibody polypeptides useful in accordance with the present invention are polypeptides which have been altered so as to exhibit additional features not found on the native polypeptide. Examples include fusion proteins. Variant polypeptides may also be referred to herein as “polypeptide analogs.” As used herein a “derivative” of an anti-αᵥβ₆ antibody or antibody polypeptide refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

Substantially, substantial: As used herein, conjugation of a protein is said not to interfere “substantially” with the ability of the protein to bind to its receptor(s) if the rate and/or amount of binding of a conjugated protein to a receptor is not less than about 40%, about 50%, about 60%, about 65%, about 70%, about 75%, about 80%, about 85%, about 90%, about 91%, about 92%, about 93%, about 94%, about 95%, about 96%, about 97%, about 98%, about 99% or about 100% or more, of the binding rate and/or amount of the corresponding cytokine, chemokine, growth factor or polypeptide hormone that has not been conjugated.

Treatment: As used herein, the terms “treatment,” “treat,” “treated” or “treating” refer to prophylaxis and/or therapy, particularly wherein the object is to prevent or slow down (lessen) an undesired physiological change or disorder, such as the progression of multiple sclerosis. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. By “subject” or “individual” or “animal” or “patient” or “mammal,” is meant any subject, particularly a mammalian subject, for whom diagnosis, prognosis, or therapy is desired. Mammalian subjects include humans and other primates, domestic animals, farm animals, and zoo, sports, or pet animals such as dogs, cats, guinea pigs, rabbits, rats, mice, horses, cattle, cows, and the like.

### Overview

The present invention relates to methods of asthma treatment and prevention using αᵥβ₆ antagonists, such as αᵥβ₆-binding antibodies. In particular, the invention relates to the discovery of a correlation between reduced expression of αᵥβ₆ and the protection from the increase in airway sensitivity seen in chronic allergen-challenged mice. This protection is associated with protection from the usual allergen-induced increase in airway epithelial mast cells.

In certain embodiments of the invention, the ligands that bind to αᵥβ₆ are antagonists of αᵥβ₆. Such antagonists include but are not limited to antibodies which specifically bind to αᵥβ₆; antibodies which specifically bind to β₆; antibodies that bind to αᵥ, antibodies that bind to ligands for αᵥβ₆; ligands for αᵥβ₆; antisense nucleic acids; and peptide, non-peptide, and peptidomimetic analogs of such ligands.

In certain such embodiments of the present invention, the ligand that binds to integrin αᵥβ₆ is an antibody that binds to integrin αᵥβ₆ (or one or more subunits of integrin αᵥβ₆), or integrin αᵥβ₆-binding fragments, variants, or derivatives thereof. Such antibodies may bind to one subunit of the integrin (e.g., antibodies that bind to an epitope located on the αᵥ subunit or to an epitope that is located on the β₆ subunit), or to both subunits (e.g., antibodies that bind to an epitope that is located in a region of the integrin heterodimer that bridges both the αᵥ and β₆ subunits). Unless specifically referring to full-sized antibodies such as naturally occurring antibodies, the term “αᵥβ₆ antibodies” encompasses full-sized antibodies as well as αᵥβ₆-binding fragments, variants, analogs, or derivatives of such antibodies, e.g., naturally occurring antibody or immunoglobulin molecules or engineered antibody molecules or fragments that bind antigen in a manner similar to antibody molecules. Antibodies can be synthetic, monoclonal, or polyclonal and can be made by techniques well known in the art. For therapeutic applications, “human” (or “humanized” or “primatized”) monoclonal antibodies having human constant and variable regions are often preferred so as to minimize the immune response of a patient against the antibody. Such antibodies can be generated by immunizing transgenic animals which contain human immunoglobulin genes (see, e.g., Jakobovits et al., Ann. N.Y. Acad. Sci. 764:525-535 (1995)). In connection with synthetic and semi-synthetic antibodies, such terms are intended to cover but are not limited to antibody fragments, isotype switched antibodies, humanized antibodies (e.g., mouse-human, human-mouse, and the like), hybrids, antibodies having plural specificities, fully synthetic antibody-like molecules, and the like.

A humanized antibody of the present invention refers to a full antibody, e.g., an antibody comprising two heavy chains and two light chains, or to an antigen-binding fragment of a full antibody such as a Fab fragment, a Fab′ fragment, a F(ab′)2 fragment or a F(v) fragment. A humanized antibody of this invention can be of any isotype and subtype, for example, IgA (e.g., IgA1 and IgA2), IgG (e.g., IgG1, IgG2, IgG3 and IgG4), IgE, IgD, IgM, wherein the light chains of the immunoglobulin may be of type kappa or lambda.

In some embodiments, the humanized antibody of the present invention may comprise a mutation (e.g., deletion, substitution or addition) at one or more (e.g., 2, 3, 4, 5, or 6) of certain positions in the heavy chain such that the effector function of the antibody (e.g., the ability of the antibody to bind to a Fc receptor or a complement factor) is altered without affecting the antibody's antigen-binding ability.

In other embodiments, the humanized antibody of this invention may contain a mutation at an amino acid residue that is a site for glycosylation such that the glycosylation site is eliminated. Such a humanized antibody may have clinically beneficial, reduced effector functions or other undesired functions while retaining its antigen-binding affinity. Mutation of a glycosylation site can also be beneficial for process development (e.g., protein expression and purification).

In certain embodiments of this invention, the humanized antibody comprises an aglycosyl light chain whose CDRs are derived from the murine 3G9 antibody. In certain embodiments, the humanized 3G9 antibody contains a light chain variable domain wherein the CDR1 region contains an asparagine (N) to serine (S) substitution at amino acid residue 26 of SEQ ID NO: 2. The murine 3G9 CDR1 region contains an asparagine at this amino acid position. However, in the humanized version of the 3G9 antibody, all five versions of the light chain (LV1, LV2, LV3, LV4 and LV5) contains a serine within the 3G9 CDR1 region at this position. Aglycosylation of this site in all light chain versions of the humanized 3G9 antibody has been shown to be beneficial for both protein expression and purification of the light chains. In certain other embodiments, the humanized 3G9 antibody contains a mutation at a glycosylation site that is normally required for normal Fc receptor binding. In certain embodiments, the humanized 3G9 antibody contains an asparagine (N) to glutamine (Q) amino acid substitution. In certain embodiments, the humanized 3G9 antibody contains the N to Q amino acid substitution in the heavy chain version 3 (HV3) produced by a recombinant vector comprising the plasmid pKJS196 (SEQ E) NO: 7). In certain embodiments, the N to Q amino acid substitution occurs at amino acid residue 319 of SEQ ID NO: 7. Aglycosylation of this site in heavy chain version 3 (HV3) of the humanized 3G9 antibody has been shown to remove a glycosylation signal required for normal Fc receptor binding without affecting the antigen-binding affinity of the humanized antibody. In certain embodiments, the humanized 3G9 antibody comprises the heavy chain version 3 (HV3) produced by a recombinant vector comprising plasmid pKJS189 (SEQ ID NO: 6) and the light chain version 5 (LV5) produced by a recombinant vector comprising plasmid pKJS195 (SEQ ID NO: 5). In certain embodiments, the humanized 3G9 antibody comprises the aglycosyl heavy chain version 3 (a-HV3) produced by a recombinant vector comprising plasmid pKJS196 (SEQ ID NO: 7) and the light chain version 5 (LV5) produced by a recombinant vector comprising plasmid pKJS195 (SEQ ID NO: 5).

In still other embodiments, the heavy or light chains can contain mutations that increase affinity or potency.

The humanized antibodies of the invention are useful for treating any clinically undesirable condition or disease (as discussed herein) that is mediated by binding of αvβ6 to its ligand, such as LAP and fibronectin. These humanized antibodies can be more potent, via higher affinity or avidity, and cation dependency or independency of binding to ligand, than previously known αvβ6 antibodies. In contrast to murine monoclonal antibodies, the humanized antibodies of this invention will not cause anti-mouse immunoglobulin antibody production in the subject's, especially a human body, but instead show a prolonged blood half-life, with a reduced frequency of adverse effects, so that it can be expected to be superior to be mouse monoclonal antibodies in the efficacy in the treatment of diseases mediated by αvβ6.

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). As will be understood by those of ordinary skill, the terms “antibody” and “immunoglobulin” comprise various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgA₁, etc. are well characterized and are known to confer functional specialization. Modified versions of each of these classes and isotypes are readily discernable to the skilled artisan in view of the instant disclosure and, accordingly, are within the scope of the instant invention.

Antibodies that bind to αᵥβ₆, or αᵥβ₆-binding fragments, variants, or derivatives thereof, that are suitable for use in the present invention include but are not limited to polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a V_{L} or V_{H} domain, fragments produced by a Fab expression library, and anti-idiotypic (anti-Id) antibodies (including, e.g., anti-Id antibodies to anti-αᵥβ₆ antibodies disclosed herein). ScFv molecules are known in the art and are described, e.g., in U.S. Pat. No. 5,892,019. Immunoglobulin or antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Antibody fragments, including single-chain antibodies, may comprise the variable region(s) alone or in combination with the entirety or a portion of the following: hinge region, C_{H}1, C_{H}2, and C_{H}3 domains. Also included in the invention are antigen-binding fragments also comprising any combination of variable region(s) with a hinge region, C_{H}1, C_{H}2, and C_{H}3 domains. Antibodies or immunospecific fragments thereof for use in the diagnostic and therapeutic methods disclosed herein may be from any animal origin including birds and mammals. Preferably, the antibodies are human, murine, rat, donkey, rabbit, goat, guinea pig, camel, llama, horse, bovine or chicken antibodies. Most preferably, the antibodies are human, humanized or primatized antibodies, or chimeric antibodies, particularly monoclonal antibodies. As used herein, “human” (or “humanized” or “primatized”) antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al. As used herein, the term “chimeric antibody” will be held to mean any antibody wherein the immunoreactive region or site is obtained or derived from a first species and the constant region (which may be intact, partial or modified in accordance with the instant invention) is obtained from a second species. In preferred embodiments the target binding region or site will be from a non-human source (e.g. mouse or primate) and the constant region is human.

Particularly preferred antibodies for use in accordance with the present invention are anti-αᵥβ₆ monoclonal antibodies such as those disclosed in Weinreb et al., J. Biol. Chem. 279(17):17875-17877 (2004) (the disclosure of which is incorporated herein by reference in its entirety), including monoclonal antibodies 6.8G6 (“8G6”) and 6.3G9 (“3G9”) disclosed therein. Additional antibodies that bind to αᵥβ₆ and that therefore are suitable for use in accordance with the present invention include antibodies (or fragments, variants or derivatives thereof) that bind to the β₆ subunit of integrin αᵥβ₆ (and that are therefore considered “anti-β₆ antibodies”), such as those disclosed in Weinacker et al., J. Cell Biol. 269:1-9 (1994), which is incorporated herein by reference in its entirety; and in U.S. Pat. No. 6,692,741 B2, which is incorporated herein by reference in its entirety, particularly at columns 2-3 and 7-8 thereof, including the monoclonal antibody designated 10D5 (ATCC deposit no. HB12382, deposited Aug. 6, 1997, American Type Culture Collection, P.O. Box 1549, Manassas, Va. 20108) (see U.S. Pat. No. 6,692,741 at col. 3, lines 7-13, and at cols. 7-8) and CSβ6 (see U.S. Pat. No. 6,692,741 at cols. 7-8). Suitable embodiments according to this aspect of the invention use αᵥβ₆ integrin-binding ligands which are αᵥβ₆-binding antibodies or αᵥβ₆ epitope-binding fragments thereof. Additional antibodies suitable for use in accordance with this aspect of the invention include, but are not limited to, the αᵥβ₆-binding monoclonal antibodies disclosed in U.S. patent application publication no. US 2005/0255102 A1, the disclosure of which is incorporated herein in its entirety, including those designated therein as 3G9, 8G6, 1A8, 2B1, 2B10, 2A1, 2E5, 1G10, 7G5, 1C5, as well as fragments, chimeras and hybrids thereof. Particularly suitable antibodies for use in accordance with the present invention are monoclonal antibodies 2B1, 3G9 and 8G6.

In some embodiments, the antibodies comprise the same heavy and light chain polypeptide sequences as an antibody produced by hybridoma 6.1A8, 6.3G9, 6.8G6, 6.2B1, 6.2B10, 6.2A1, 6.2E5, 7.1G10, 7.7G5, or 7.1C5. Particularly suitable antibodies for use in accordance with the present invention are monoclonal antibodies that comprise the same heavy and light chain polypeptide sequences as 2B1 antibodies produced by hybridoma 6.2B1 (ATCC deposit no. PTA-3646, deposited Aug. 16, 2001, American Type Culture Collection, P.O. Box 1549, Manassas, Va. 20108), 8G6 antibodies produced by hybridoma 6.8G6 (ATCC deposit no. PTA-3645, deposited Aug. 16, 2001, American Type Culture Collection, P.O. Box 1549, Manassas, Va. 20108) and 3G9 antibodies produced by hybridoma 6.3G9 (ATCC deposit no. PTA-3649, deposited Aug. 16, 2001, American Type Culture Collection, P.O. Box 1549, Manassas, Va. 20108) (see published U.S. Appl. No. US 2005/0255102 A1, the disclosure of which is incorporated herein by reference in its entirety, particularly at page 1, paragraph 0008; at page 2, paragraphs 0032 and 0036; and in the Examples at pages 6-14), and the antibody designated as 10D5 (the hybridoma secreting which antibody was deposited on Aug. 6, 1997, as ATCC deposit no. HB12382, American Type Culture Collection, P.O. Box 1549, Manassas, Va. 20108) (see U.S. Pat. No. 6,692,741, the disclosure of which is incorporated herein by reference in its entirety, particularly at col. 3, lines 7-13, and at cols. 7-8).

In other related embodiments, the monoclonal antibodies used in accordance with the present invention are chimeric antibodies, i.e., those in which a cognate antibody from one species (e.g., murine, rat or rabbit) is altered by recombinant DNA technology such that part or all of the hinge and/or constant regions of the heavy and/or light chains are replaced with the corresponding components of an antibody from another species (e.g., human). Generally, the variable domains of the engineered antibody remain identical or substantially so to the variable domains of the cognate antibody. Such an engineered antibody is called a chimeric antibody and is less antigenic than the cognate antibody when administered to an individual of the species from which the hinge and/or constant region is derived (e.g., a human). Methods of making chimeric antibodies are well known in the art.

In other related embodiments, the monoclonal antibodies used in accordance with the present invention are fully human antibodies. Methods for producing such fully human monoclonal antibodies are well known in the art (see, e.g., US 2005/0255102 A1 at page 4, paragraphs 0069-0070, which are incorporated herein by reference).

In other related embodiments, the monoclonal antibodies used in accordance with the present invention are humanized versions of cognate anti-αᵥβ₆ antibodies derived from other species. A humanized antibody is an antibody produced by recombinant DNA technology, in which some or all of the amino acids of a human immunoglobulin light or heavy chain that are not required for antigen binding (e.g., the constant regions and the framework regions of the variable domains) are used to substitute for the corresponding amino acids from the light or heavy chain of the cognate, nonhuman antibody. By way of example, a humanized version of a murine antibody to a given antigen has, on both of its heavy and light chain: (a) constant regions of a human antibody; (b) framework regions from the variable domains of a human antibody; and (c) CDRs from the murine antibody. When necessary, one or more residues in the human framework regions can be changed to residues at the corresponding positions in the murine antibody so as to preserve the binding affinity of the humanized antibody to the antigen. This change is sometimes called “back mutation.” Humanized antibodies generally are less likely to elicit an immune response in humans as compared to chimeric human antibodies because the former contain considerably fewer non-human components. Methods for producing such humanized monoclonal antibodies are well known in the art (see, e.g., US 2005/0255102 A1 at pages 4-5, paragraphs 0072-0077, which are incorporated herein by reference).

In one embodiment, the present invention relates to humanized monoclonal antibodies having binding specificity for αvβ6 integrins for use in methods of treating asthma. More particularly, the antibody comprises heavy and light chain variable domains of SEQ ID NO: 1 and SEQ ID NO: 2, respectively. Such humanized antibodies are derived from the humanization of the murine 3G9 antibody, in certain embodiments, the humanized antibodies comprise a heavy chain whose complementarity determining regions (CDR) 1, 2 and 3 comprise amino acid residues 31-35, 50-65 and 98-109, respectively, of SEQ ID NO: 1. In certain embodiments, the humanized antibodies comprise a light chain whose CDRs 1, 2 and 3 comprise amino acid residues 24-35, 51-57 and 90-98, respectively, of SEQ ID NO: 2. In certain embodiments, the humanized antibodies comprise a heavy chain whose framework regions (FR) 1, 2, 3 and 4 comprise amino acid residues 1-30, 36-49, 66-97 and 110-120, respectively, of SEQ ID NO: 1. In certain embodiments, the humanized antibodies comprise a light chain whose framework regions (FR) 1, 2, 3 and 4 comprise amino acid residues 1-23, 36-50, 58-89 and 99-108, respectively, of SEQ ID NO: 2.

In certain embodiments, the humanized antibodies used in the therapeutic methods for controlling, treating, preventing or ameliorating the symptoms of asthma comprise at least one of the following amino acid substitutions in the heavy chain consisting of Q3M and N74S of SEQ ID NO: 1. In certain embodiments, the humanized antibodies comprise at least one of the following amino acid substitutions in the light chain consisting of E1Q, L47W, 158V, A60V and Y87F of SEQ ID NO: 2.

In certain embodiments, the humanized antibody used in the therapeutic methods of this invention comprises a heavy chain version 1 (“HV1”) wherein the heavy chain consists of amino acid substitutions Q3M and N74S of SEQ ID NO: 1. In certain embodiments, the humanized antibody comprises a heavy chain version 2 (“HV2”) wherein the heavy chain consists of amino acid substitution N74S of SEQ ID NO: 1. In certain embodiments, the humanized antibody comprises a heavy chain version 3 (“HV3”) wherein the heavy chain consists of SEQ ID NO: 1.

In some embodiments, the humanized antibody used in the treatment methods disclosed herein comprises a light chain version 1 (“LV1”) wherein the light chain consists of amino acid substitutions L47W, 158 V, A60V and Y87F of SEQ ID NO: 2. In certain embodiments, the humanized antibody comprises a light chain version 2 (“LV2”) wherein the light chain consists of amino acid substitutions L47W and 158V of SEQ ID NO: 2. In certain embodiments, the humanized antibody comprises a light chain version 3 (“LV3”) wherein the light chain consists of amino acid substitution L47W of SEQ ID NO: 2. In certain embodiments, the humanized antibody comprises a light chain version 4 (“LV4”) wherein the light chain consists of amino acid substitutions E1Q and L47W of SEQ ID NO: 2. In certain embodiments, the humanized antibody comprises a light chain version 5 (“LV5”) wherein the light chain consists of SEQ ID NO: 2.

In certain embodiments, the humanized antibody comprises a heavy and light chain variable domain comprising HV3 wherein the heavy chain consists of SEQ ID NO: 1 and LV5 wherein the light chain consists of SEQ ID NO: 2.

In certain embodiments, the humanized antibodies have CDRs derived from the murine 6.3G9 antibody (ATCC Accession No. PTA-3649).

In related embodiments, the present invention also relates to the use of humanized monoclonal antibodies having binding specificity for αvβ6 integrins for the treatment of asthma, wherein the antibodies comprises a heavy and light chain variable domains of SEQ ID NO: 3 and SEQ ID NO: 4. Such humanized antibodies are derived from the humanization of the murine 8G6 antibody. In certain embodiments, the humanized antibodies comprise a heavy chain whose complementarity determining regions (CDR) 1, 2 and 3 comprise amino acid residues (i.e., with the exception of some conservative variations) 31-35, 50-66 and 99-115, respectively, of SEQ ID NO: 3. In certain embodiments, the humanized antibodies comprise a light chain whose CDRs 1, 2 and 3 comprise amino acid residues 24-38, 54-60 and 93-101, respectively, of SEQ ID NO: 4. In certain embodiments, the humanized antibodies comprise a heavy chain whose framework regions (FR) 1, 2, 3 and 4 comprise amino acid residues 1-30, 36-49, 67-98 and 116-126, respectively, of SEQ ID NO: 3. In certain embodiments, the humanized antibodies comprise a light chain whose FR 1, 2, 3 and 4 comprise amino acid residues 1-23, 39-53, 61-92 and 102-111, respectively, of SEQ ID NO: 4.

In certain embodiments, the humanized antibodies used in the methods described herein comprise at least one of the following amino acid substitutions in the heavy chain consisting of A24G, G26S, Q39L, M48I, V68A, R72V and T74K of SEQ ID NO: 3. In certain embodiments, the humanized antibodies comprise at least one of the following amino acid substitutions in the light chain consisting of E1D, L46F and Y49K of SEQ ID NO: 4.

In certain embodiments, the humanized antibody used in the methods described herein comprises a heavy chain version 1 (“HV1′”) wherein the heavy chain consists of amino acid substitutions A24G, G26S, Q39L, M48I, V68A, R72V and T74K of SEQ ID NO: 3. In certain embodiments, the humanized antibody comprises a heavy chain version 2 (“HV2′”) wherein the heavy chain consists of amino acid substitutions M48I, V68A, R72V and T74K of SEQ ID NO: 3. In certain embodiments, the humanized antibody comprises a heavy chain version 3 (“HV3′”) wherein the heavy chain consists of amino acid substitutions V68A, R72V and T74K of SEQ ID NO: 3.

In certain embodiments, the humanized antibody used in the treatment of asthma comprises a light chain version 1 (“LV1′”) wherein the light chain consists of amino acid substitutions E1D, L46F and Y49K of SEQ ID NO: 4. In certain embodiments, the humanized antibody comprises a light chain version 2 (“LV2′”) wherein the light chain consists of amino acid substitution L46F and Y49K of SEQ ID NO: 4. In certain embodiments, the humanized antibody comprises a light chain version 3 (“LV3′”) wherein the light chain consists of amino acid substitution Y49K of SEQ ID NO: 4.

In certain embodiments, the humanized antibodies that are used in the therapeutic methods described herein have CDRs derived from the murine 6.8G6 antibody. In certain embodiments, the humanized antibodies can compete for binding to αvβ6 with murine 8G6 antibody.

The present invention also embraces the use of humanized antibodies that bind to the same epitope as any of the above-described antibodies for the treatment, prevention or amelioration of the symptoms of asthma.

The present invention also embraces use of humanized antibodies produced by a recombinant vector comprising a nucleic acid encoding said antibodies for the treatment of asthma. In certain embodiments, the recombinant vector may be a plasmid selected from the group consisting of pKJS195 (SEQ ID NO: 5), pKJS189 (SEQ ID NO: 6) and pKJS196 (SEQ ID NO: 7).

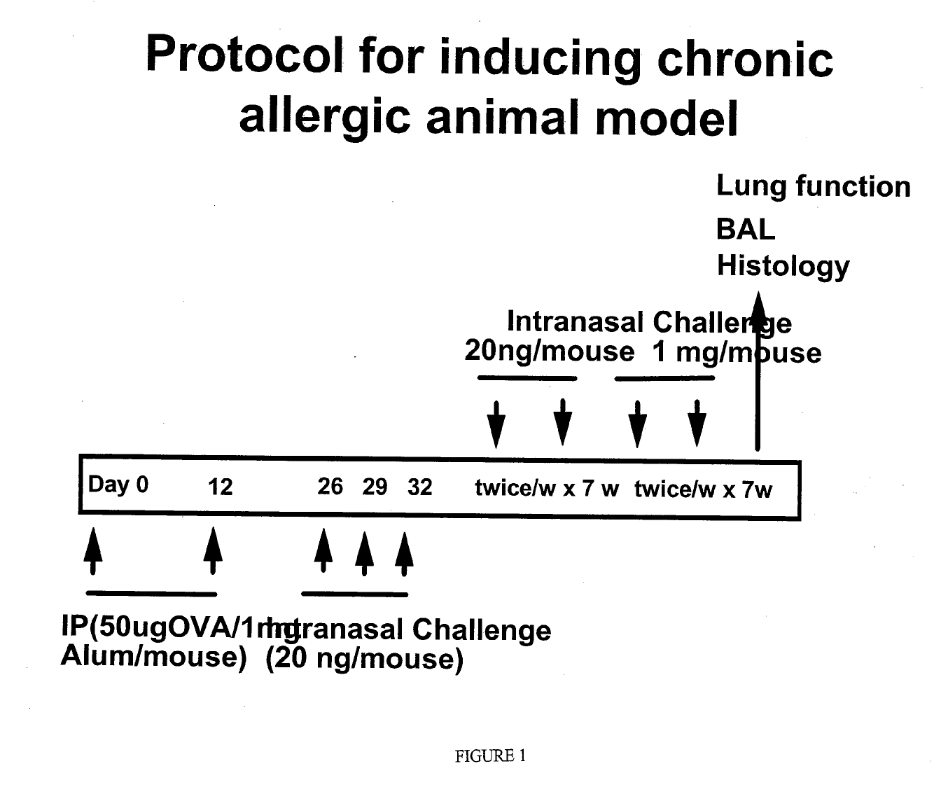

As described in PCT publication No. WO2007/008712 and its counterpart U.S. Application, Serial No. U.S. Ser. No. 11/483,190 (each of which is incorporated herein by reference in its entirety), exemplary humanized versions of the chimeric antibodies 3G9 and 8G6, have been generated. For the 3G9 antibody, this involved the cloning of the murine 3G9 variable heavy and light chain regions as described in the Examples herein. The cDNAs encoding the murine 3G9 variable regions of the light and heavy chains were then used to construct vectors for expression of murine-human chimeras in which the murine 3G9 variable regions were linked to human IgG1 (for heavy chain) and human kappa (for light chain) constant regions. Expression of the light chain and heavy chain 3G9 expression vectors following transfection into 293-EBNA cells indicated that chimeric 3G9 transfected cells synthesized and efficiently assembled the heavy and light chains and secreted antibody (see Example 2 of PCT publication No. WO2007/008712 and its counterpart U.S. Application, Serial No. U.S. Ser. No. 11/483,190). In addition, an aglycosyl mutant form of the chimeric 3G9 antibody was also created. An amino acid substitution of an asparagine (N) to a serine (S) within an N-linked glycosylation site in the first CDR of the light chain of 3G9 was shown to greatly improve protein expression and purification without altering binding affinity (see for example FIG. 1 of PCT publication No. WO2007/008712 and its counterpart U.S. Application, Serial No. U.S. Ser. No. 11/483,190).

In order to produce humanized 3G9 antibodies, the human acceptor framework domains were chosen by homology matching to human germline sequences. For the light chain, the human L6 acceptor frameworks were found to be most homologous and for the heavy chain, the human 3-7 acceptor frameworks were found to most homologous, as described in Example 3. Using these chosen human acceptor frameworks, the light and heavy chain variable domains were designed and a number of variants/versions of each were generated and expressed (see Example 4 of PCT publication No. WO2007/008712 and its counterpart U.S. Application, Serial No. U.S. Ser. No. 11/483,190).

Exemplary humanized 3G9 antibodies that can be used in the methods of this invention include those comprising a heavy chain variable domain of SEQ ID NO: 1 and light chain variable domain of SEQ ID NO: 2.

SEQ ID NO: 1:
EVQLVESGGGLVQPGGSLRLSCAASGFTFSRYWMSWVRQAPGKGLE
WVASISSGGRMYYPDTVKGRFTISRDNAKNSLYLQMNSLRAEDTAV
YYCARGSIYDGYYVFPYWGQGTLVTVSS
SEQ ID NO: 2:
EIVLTQSPATLSLSPGERATLSCSASSSVSSSYLYWYQQKPGQAPRLLIY
STSNLASGIPARFSGSGSGTDFTLTISSLEPEDFAVYYCHQWSTYPPTFG
GGTKVEIK

Different variants/versions of the 3G9 heavy and light chains were generated with different degrees of back mutations to determine which combination produced the best humanized antibody with superior binding affinity and blocking activity to αvβ6. Of the five different versions of light chains and the three different versions of heavy chains generated, the pairing of 3G9 heavy chain version 3 (HV3) with 3G9 light chain version 5 (LV5) generated the best humanized antibody (see Example 4 of PCT publication No. WO2007/008712 and its counterpart U.S. Application, Serial No. U.S. Ser. No. 11/483,190). This humanized 3G9 version 5 (H3/L5) antibody is produced by expression of the recombinant vector for heavy chain version 3 (H3) comprising the plasmid pKJS189 (SEQ ID NO: 6) in combination with the recombinant vector for light chain version 5 (LV5) comprising the plasmid pKJS195 (SEQ ID NO: 5).

| SEQ ID NO: 6: | |
|---|---|
| 1263 | ATG GAC TTC GGC CTG AGC TGG GTG TTC CTG GTG CTG GTG CTG |
| | AAG GGC GTG CAG TGC GAG GTG CAG CTG GTG GAG AGC GGC GGC |
| 1 | Met Asp Phe Gly Leu Ser Trp Val Phe Leu Val Leu Val Leu |
| | Lys Gly Val Gln Cys Glu Val Gln Leu Val Glu Ser Gly Gly |
| 1347 | GGC CTG GTG CAG CCC GGC GGC AGC CTG AGG CTG AGC TGC GCC |
| | GCC AGC GGC TTC ACC TTC AGC CGC TAC GTG ATG AGC TGG GTG |
| 29 | Gly Leu Val Gln Pro Gly Gly Ser Leu Arg Leu Ser Cys Ala |
| | Ala Ser Gly Phe Thr Phe Ser Arg Tyr Val Met Ser Trp Val |
| 1431 | CGC CAG GCC CCC GGC AAG GGC CTG GAG TGG GTG GCC AGC ATC |
| | AGC AGC GGA GGC CGC ATG TAC TAC CCC GAC ACC GTG AAG GGC |
| 57 | Arg Gln Ala Pro Gly Lys Gly Leu Glu Trp Val Ala Ser Ile |
| | Ser Ser Gly Gly Arg Met Tyr Tyr Pro Asp Thr Val Lys Gly |
| 1515 | CGC TTC ACC ATC AGC CGC GAC AAC GCC AAG AAC AGC CTG TAC |
| | CTG CAG ATG AAC AGC CTG CGC GCC GAG GAC ACC GCC GTG TAC |
| 85 | Arg Phe Thr Ile Ser Arg Asp Asn Ala Lys Asn Ser Leu Tyr |
| | Leu Gln Met Asn Ser Leu Arg Ala Glu Asp Thr Ala Val Tyr |
| 1599 | TAC TGC GCC CGC GGC AGC ATC TAC GAC GGC TAC TAC GTG TTC |
| | CCC TAC TGG GGC CAG GGC ACC CTG GTG ACC GTG AGC TCC GCC |
| 113 | Tyr Cys Ala Arg Gly Ser Ile Tyr Asp Gly Tyr Tyr Val Phe |
| | Pro Tyr Trp Gly Gln Gly Thr Leu Val Thr Val Ser Ser Ala |
| 1683 | AGC ACC AAG GGC CCC AGC GTG TTC CCC CTG GCC CCC AGC AGC |
| | AAG AGC ACC AGC GGC GGC ACC GCC GCC CTG GGC TGC CTG GTG |
| 141 | Ser Thr Lys Gly Pro Ser Val Phe Pro Leu Ala Pro Ser Ser |
| | Lys Ser Thr Ser Gly Gly Thr Ala Ala Leu Gly Cys Leu Val |
| 1767 | AAG GAC TAC TTC CCC GAA CCG GTG ACG GTG TCG TGG AAC TCA |
| | GGC GCC CTG ACC AGC GGC GTG CAC ACC TTC CCG GCT GTC CTA |
| 169 | Lys Asp Tyr Phe Pro Glu Pro Val Thr Val Ser Trp Asn Ser |
| | Gly Ala Leu Thr Ser Gly Val His Thr Phe Pro Ala Val Leu |
| 1851 | CAG TCC TCA GGA CTC TAC TCC CTC AGC AGC GTG GTG ACC GTG |
| | CCC TCC AGC AGC TTG GGC ACC CAG ACC TAC ATC TGC AAC GTG |
| 197 | Gln Ser Ser Gly Leu Tyr Ser Leu Ser Ser Val Val Thr Val |
| | Pro Ser Ser Ser Leu Gly Thr Gln Thr Tyr Ile Cys Asn Val |
| 1935 | AAT CAC AAG CCC AGC AAC ACC AAG GTG GAC AAG AAA GTT GAG |
| | CCC AAA TCT TGT GAC AAG ACT CAC ACA TGC CCA CCG TGC CCA |
| 225 | Asn His Lys Pro Ser Asn Thr Lys Val Asp Lys Lys Val Glu |
| | Pro Lys Ser Cys Asp Lys Thr His Thr Cys Pro Pro Cys Pro |
| 2019 | GCA CCT GAA CTC CTG GGG GGA CCG TCA GTC TTC CTC TTC CCC |
| | CCA AAA CCC AAG GAC ACC CTC ATG ATC TCC CGG ACC CCT GAG |
| 253 | Ala Pro Glu Leu Leu Gly Gly Pro Ser Val Phe Leu Phe Pro |
| | Pro Lys Pro Lys Asp Thr Leu Met Ile Ser Arg Thr Pro Glu |
| 2103 | GTC ACA TGC GTG GTG GTG GAC GTG AGC CAC GAA GAC CCT GAG |
| | GTC AAG TTC AAC TGG TAC GTG GAC GGC GTG GAG GTG CAT AAT |
| 281 | Val Thr Cys Val Val Val Asp Val Ser His Glu Asp Pro Glu |
| | Val Lys Phe Asn Trp Tyr Val Asp Gly Val Glu Val His Asn |
| 2187 | GCC AAG ACA AAG CCG CGG GAG GAG CAG TAC AAC AGC ACG TAC |
| | CGT GTG GTC AGC GTC CTC ACC GTC CTG CAC CAG GAC TGG CTG |
| 309 | Ala Lys Thr Lys Pro Arg Glu Glu Gln Tyr Asn Ser Thr Tyr |
| | Arg Val Val Ser Val Leu Thr Val Leu His Gln Asp Trp Leu |
| 2271 | AAT GGC AAG GAG TAC AAG TGC AAG GTC TCC AAC AAA GCC CTC |
| | CCA GCC CCC ATC GAG AAA ACC ATC TCC AAA GCC AAA GGG CAG |
| 337 | Asn Gly Lys Glu Tyr Lys Cys Lys Val Ser Asn Lys Ala Leu |
| | Pro Ala Pro Ile Glu Lys Thr Ile Ser Lys Ala Lys Gly Gln |
| 2355 | CCC CGA GAA CCA CAG GTG TAC ACC CTG CCC CCA TCC CGG GAT |
| | GAG CTG ACC AAG AAC CAG GTC AGC CTG ACC TGC CTG GTC AAA |
| 365 | Pro Arg Glu Pro Gln Val Tyr Thr Leu Pro Pro Ser Arg Asp |
| | Glu Leu Thr Lys Asn Gln Val Ser Leu Thr Cys Leu Val Lys |
| 2439 | GGC TTC TAT CCC AGC GAC ATC GCC GTG GAG TGG GAG AGC AAT |
| | GGG CAG CCG GAG AAC AAC TAC AAG ACC ACG CCT CCC GTG TTG |
| 393 | Gly Phe Tyr Pro Ser Asp Ile Ala Val Glu Trp Glu Ser Asn |
| | Gly Gln Pro Glu Asn Asn Tyr Lys Thr Thr Pro Pro Val Leu |
| 2523 | GAC TCC GAC GGC TCC TTC TTC CTC TAC AGC AAG CTC ACC GTG |
| | GAC AAG AGC AGG TGG CAG CAG GGG AAC GTC TTC TCA TGC TCC |
| 421 | Asp Ser Asp Gly Ser Phe Phe Leu Tyr Ser Lys Leu Thr Val |
| | Asp Lys Ser Arg Trp Gln Gln Gly Asn Val Phe Ser Cys Ser |
| 2607 | GTG ATG CAT GAG GCT CTG CAC AAC CAC TAC ACG CAG AAG AGC |
| | CTC TCC CTG TCT CCC GGT |
| 449 | Val Met His Glu Ala Leu His Asn His Tyr Thr Gln Lys Ser |
| | Leu Ser Leu Ser Pro Gly |
| SEQ ID NO: 5: | |
| 1263 | ATG GAC TTC CAG GTG CAG ATC TTC AGC TTC CTG CTG ATC AGC |
| | GTG AGC GTG ATC ATG AGC CGC GGC GAG ATC GTG CTG ACC CAG |
| 1 | Met Asp Phe Gln Val Gln Ile Phe Ser Phe Leu Leu Ile Ser |
| | Val Ser Val Ile Met Ser Arg Gly Glu Ile Val Leu Thr Gln |
| 1347 | AGC CCC GCC ACC CTG AGC CTG AGC CCC GGC GAG AGG GCC ACC |
| | CTG AGC TGC AGC GCC AGC AGC AGC GTG AGC AGC AGC TAC CTG |
| 29 | Ser Pro Ala Thr Leu Ser Leu Ser Pro Gly Glu Arg Ala Thr |
| | Leu Ser Cys Ser Ala Ser Ser Ser Val Ser Ser Ser Tyr Leu |
| 1431 | TAC TGG TAC CAG CAG AAG CCC GGC CAG GCC CCC AGG CTG CTG |
| | ATC TAC AGC ACC AGC AAC CTG GCC AGC GGC ATC CCC GCC CGC |
| 56 | Tyr Trp Tyr Gln Gln Lys Pro Gly Gln Ala Pro Arg Leu Leu |
| | Ile Tyr Ser Thr Ser Asn Leu Ala Ser Gly Ile Pro Ala Arg |
| 1515 | TTC AGC GGC AGC GGC AGC GGC ACC GAC TTC ACC CTG ACC ATC |
| | AGC AGC CTG GAG CCC GAG GAC TTC GCC GTG TAC TAC TGC CAC |
| 83 | Phe Ser Gly Ser Gly Ser Gly Thr Asp Phe Thr Leu Thr Ile |
| | Ser Ser Leu Glu Pro Glu Asp Phe Ala Val Tyr Tyr Cys His |
| 1599 | CAG TGG AGC ACC TAC CCC CCC ACC TTC GGC GGC GGC ACC AAG |
| | GTG GAG ATC AAG CGT ACG GTG GCT GCA CCA TCT GTC TTC ATC |
| 110 | Gln Trp Ser Thr Tyr Pro Pro Thr Phe Gly Gly Gly Thr Lys |
| | Val Glu Ile Lys Arg Thr Val Ala Ala Pro Ser Val Phe Ile |
| 1633 | TTC CCG CCA TCT GAT GAG CAG TTG AAA TCT GGA ACT GCC TCT |
| | GTT GTG TGC CTG CTG AAT AAC TTC TAT CCC AGA GAG GCC AAA |
| 137 | Phe Pro Pro Ser Asp Glu Gln Leu Lys Ser Gly Thr Ala Ser |
| | Val Val Cys Leu Leu Asn Asn Phe Tyr Pro Arg Glu Ala Lys |
| 1767 | GTA CAG TGG AAG GTG GAT AAC GCC CTC CAA TCG GGT AAC TCC |
| | CAG GAG AGT GTC ACA GAG CAG GAC AGC AAG GAC AGC ACC TAC |
| 164 | Val Gln Trp Lys Val Asp Asn Ala Leu Gln Ser Gly Asn Ser |
| | Gln Glu Ser Val Thr Glu Gln Asp Ser Lys Asp Ser Thr Tyr |
| 1851 | AGC CTC AGC AGC ACC CTG ACG CTG AGC AAA GCA GAC TAC GAG |
| | AAA CAC AAA GTC TAC GCC TGC GAA GTC ACC CAT CAG GGC CTG |
| 191 | Ser Leu Ser Ser Thr Leu Thr Leu Ser Lys Ala Asp Tyr Glu |
| | Lys His Lys Val Tyr Ala Cys Glu Val Thr His Gln Gly Leu |
| 1935 | AGC TCG CCC GTC ACA AAG AGC TTC AAC AGG GGA GAG TGT |
| 218 | Ser Ser Pro Val Thr Lys Ser Phe Asn Arg Gly Glu Cys |

Another version of the humanized 3G9 version 5 (H3/L5) antibody was also generated in which the heavy chain was mutated to remove a glycosylation site in the constant region, which has been shown to be required for normal Fc receptor binding (see Example 5 of PCT publication No. WO2007/008712 and its counterpart U.S. Application, Serial No. U.S. Ser. No. 11/483,190). This aglycosyl form of humanized 3G9 antibody (a-H3/L5) is produced by substituting an amino acid residue asparagine (N) with a glutamine (Q) in the constant region of heavy chain version 3 (H3). The aglycosyl humanized 3G9 (a-H3/L5) antibody is produced by expression of the recombinant vector for aglycosyl heavy chain version 3 (a-H3) comprising the plasmid pKJS196 (SEQ ID NO: 7) in combination with the recombinant vector for light chain version 5 (L5) comprising the plasmid pKJS195 (SEQ ID NO: 5; see above).

SEQ ID NO: 7:
ATG GAC TTC GGC CTG AGC TGG GTG TTC CTG GTG CTG GTG CTG
AAG GGC GTG CAG TGC GAG GTG CAG CTG GTG GAG AGC GGC GGC
Met Asp Phe Gly Leu Ser Trp Val Phe Leu Val Leu Val Leu
Lys Gly Val Gln Cys Glu Val Gln Leu Val Glu Ser Gly Gly
GGC CTG GTG CAG CCC GGC GGC AGC CTG AGG CTG AGC TGC GCC
GCC AGC GGC TTC ACC TTC AGC CGC TAC GTG ATG AGC TGG GTG
Gly Leu Val Gln Pro Gly Gly Ser Leu Arg Leu Ser Cys Ala
Ala Ser Gly Phe Thr Phe Ser Arg Tyr Val Met Ser Trp Val
CGC CAG GCC CCC GGC AAG GGC CTG GAG TGG GTG GCC AGC ATC
AGC AGC GGA GGC CGC ATG TAC TAC CCC GAC ACC GTG AAG GGC
Arg Gln Ala Pro Gly Lys Gly Leu Glu Trp Val Ala Ser Ile
Ser Ser Gly Gly Arg Met Tyr Tyr Pro Asp Thr Val Lys Gly
CGC TTC ACC ATC AGC CGC GAC AAC GCC AAG AAC AGC CTG TAC
CTG CAG ATG AAC AGC CTG CGC GCC GAG GAC ACC GCC GTG TAC
Arg Phe Thr Ile Ser Arg Asp Asn Ala Lys Asn Ser Leu Tyr
Leu Gln Met Asn Ser Leu Arg Ala Glu Asp Thr Ala Val Tyr
TAC TGC GCC CGC GGC AGC ATC TAC GAC GGC TAC TAC GTG TTC
CCC TAC TGG GGC CAG GGC ACC CTG GTG ACC GTG AGC TCC GCC
Tyr Cys Ala Arg Gly Ser Ile Tyr Asp Gly Tyr Tyr Val Phe
Pro Tyr Trp Gly Gln Gly Thr Leu Val Thr Val Ser Ser Ala
AGC ACC AAG GGC CCC AGC GTG TTC CCC CTG GCC CCC AGC AGC
AAG AGC ACC AGC GGC GGC ACC GCC GCC CTG GGC TGC CTG GTG
Ser Thr Lys Gly Pro Ser Val Phe Pro Leu Ala Pro Ser Ser
Lys Ser Thr Ser Gly Gly Thr Ala Ala Leu Gly Cys Leu Val
AAG GAC TAC TTC CCC GAA CCG GTG ACG GTG TCG TGG AAC TCA
GGC GCC CTG ACC AGC GGC GTG CAC ACC TTC CCG GCT GTC CTA
Lys Asp Tyr Phe Pro Glu Pro Val Thr Val Ser Trp Asn Ser
Gly Ala Leu Thr Ser Gly Val His Thr Phe Pro Ala Val Leu
CAG TCC TCA GGA CTC TAC TCC CTC AGC AGC GTG GTG ACC GTG
CCC TCC AGC AGC TTG GGC ACC CAG ACC TAC ATC TGC AAC GTG
Gln Ser Ser Gly Leu Tyr Ser Leu Ser Ser Val Val Thr Val
Pro Ser Ser Ser Leu Gly Thr Gln Thr Tyr Ile Cys Asn Val
AAT CAC AAG CCC AGC AAC ACC AAG GTG GAC AAG AAA GTT GAG
CCC AAA TCT TGT GAC AAG ACT CAC ACA TGC CCA CCG TGC CCA
Asn His Lys Pro Ser Asn Thr Lys Val Asp Lys Lys Val Glu
Pro Lys Ser Cys Asp Lys Thr His Thr Cys Pro Pro Cys Pro
GCA CCT GAA CTC CTG GGG GGA CCG TCA GTC TTC CTC TTC CCC
CCA AAA CCC AAG GAC ACC CTC ATG ATC TCC CGG ACC CCT GAG
Ala Pro Glu Leu Leu Gly Gly Pro Ser Val Phe Leu Phe Pro
Pro Lys Pro Lys Asp Thr Leu Met Ile Ser Arg Thr Pro Glu
GTC ACA TGC GTG GTG GTG GAC GTG AGC CAC GAA GAC CCT GAG
GTC AAG TTC AAC TGG TAC GTG GAC GGC GTG GAG GTG CAT AAT
Val Thr Cys Val Val Val Asp Val Ser His Glu Asp Pro Glu
Val Lys Phe Asn Trp Tyr Val Asp Gly Val Glu Val His Asn
GCC AAG ACA AAG CCG CGG GAG GAG CAG TAC CAG AGC ACG TAC
CGT GTG GTC AGC GTC CTC ACC GTC CTG CAC CAG GAC TGG CTG
Ala Lys Thr Lys Pro Arg Glu Glu Gln Tyr Gln Ser Thr Tyr
Arg Val Val Ser Val Leu Thr Val Leu His Gln Asp Trp Leu
AAT GGC AAG GAG TAC AAG TGC AAG GTC TCC AAC AAA GCC CTC
CCA GCC CCC ATC GAG AAA ACC ATC TCC AAA GCC AAA GGG CAG
Asn Gly Lys Glu Tyr Lys Cys Lys Val Ser Asn Lys Ala Leu
Pro Ala Pro Ile Glu Lys Thr Ile Ser Lys Ala Lys Gly Gln
CCC CGA GAA CCA CAG GTG TAC ACC CTG CCC CCA TCC CGG GAT
GAG CTG ACC AAG AAC CAG GTC AGC CTG ACC TGC CTG GTC AAA
Pro Arg Glu Pro Gln Val Tyr Thr Leu Pro Pro Ser Arg Asp
Glu Leu Thr Lys Asn Gln Val Ser Leu Thr Cys Leu Val Lys
GGC TTC TAT CCC AGC GAC ATC GCC GTG GAG TGG GAG AGC AAT
GGG CAG CCG GAG AAC AAC TAC AAG ACC ACG CCT CCC GTG TTG
Gly Phe Tyr Pro Ser Asp Ile Ala Val Glu Trp Glu Ser Asn
Gly Gln Pro Glu Asn Asn Tyr Lys Thr Thr Pro Pro Val Leu
GAC TCC GAC GGC TCC TTC TTC CTC TAC AGC AAG CTC ACC GTG
GAC AAG AGC AGG TGG CAG CAG GGG AAC GTC TTC TCA TGC TCC
Asp Ser Asp Gly Ser Phe Phe Leu Tyr Ser Lys Leu Thr Val
Asp Lys Ser Arg Trp Gln Gln Gly Asn Val Phe Ser Cys Ser
GTG ATG CAT GAG GCT CTG CAC AAC CAC TAC ACG CAG AAG AGC
CTC TCC CTG TCT CCC GGT
Val Met His Glu Ala Leu His Asn His Tyr Thr Gln Lys Ser
Leu Ser Leu Ser Pro Gly

Similar approaches were used in the design of the humanized 8G6 antibody (see Example 7 of PCT publication No. WO2007/008712 and its counterpart U.S. Application, Serial No. U.S. Ser. No. 11/483,190). Three versions of the 8G6 variable light chain and variable heavy chain were designed, with the first version containing the most back mutations and the third version containing the fewest (the most “humanized”) (see Example 5 of PCT publication No. WO2007/008712 and its counterpart U.S. Application, Serial No. U.S. Ser. No. 11/483,190).

SEQ ID NO: 8
(hu8G6 version 1 light chain):
DIVLTQSP ATLSLSPGERATLSCRASQSVSTSSYSYMYWYQQKPGQAP
RFLIKYASNLESGIP ARFSGSGSGTDFTLTISSLEPEDFAVYYCQHNWE
IPFTFGGGTKVEIK
SEQ ID NO: 9
(hu8G6 version 2 light chain):
EIVLTQSPATLSLSPGERATLSCRASQSVSTSSYSYMYWYQQKPGQAP
RFLIKYASNLESGIPARFSGSGSGTDFTLTISSLEPEDFAVYYCQHNWEI
PFTFGGGTKVEIK
SEQ ID NO: 10
(hu8G6 version 3 light chain):
EFVLTQSP ATLSLSPGERATLSCRASQSVSTSSYSYMYWYQQKPGQ
APRLLIKYASNLESGIP ARFSGSGSGTDFTLTISSLEPEDF AVYYCQH
NWEIPFTFGGGTKVEK
SEQ ID NO: 11
(hu8G6 version 1 heavy chain):
QVQLVQSGAEVKKPGASVKVSCKGSSYTFTDYAMHWVRLAPGQGLE
WIGVISTYYGNTNYNQKFKGRATMTVDKSISTAYMELSRLRSDDTAV
YYCARGGLRRGDRPSLRYAMDYWGQGTLVTVSS
SEQ ID NO: 12
(hu8G6 version 2 heavy chain)
QVQLVQSGAEVKKPGASVKVSCKASGYTFTDYAMHWVRQAPGQGL
EWIGVISTYYGNTNYNQKFKGRATMTVDKSISTAYMELSRLRSDDTA
VYYCARGGLRRGDRPSLRYAMDYWGQGTLVTVSS
SEQ ID NO: 13
(hu8G6 version 3 heavy chain):
QVQLVQSGAEVKKPGASVKVSCKASGYTFTDYAMHWVRQAPGQGLEWMGV
ISTYYGNTNYNQKFKGRATMTVDKSISTAYMELSRLRSDDTA
VYYCARGGLRRGDRPSLRYAMDYWGQGTLVTVSS.

In some embodiments, the antibodies comprise a heavy chain whose complementarity determining regions (CDR) 1, 2 and 3 consist essentially (i.e., with the exception of some conservative variations) of the sequences shown in Table 1 below, In certain such embodiments, the antibodies comprise a heavy chain whose CDR1 consists essentially of any one of SEQ ID NOs: 14-18; whose CDR2 consists essentially of any one of SEQ ID NOs: 19-24; and whose CDR3 consists essentially of any one of SEQ ID NOs:25-30; and/or a light chain whose CDRs 1, 2 and 3 consist essentially of any one of the sequences of SEQ ID NOs:31-36, 37-40, and 41-46, respectively. In still other less preferredembodiments, the hu8G6 heavy chain version 1, 2, and 3, respectively, contains a glutamine (Q) at residue 110 instead of and arginine (R), such that the sequences of hu8G6 version 1, version 2, and version 3 are SEQ ID NOs:90, 91 and 92, respectively. This is because in initial studies sequencing multiple isolates revealed a polymorphism at position 110 of the 8G6 heavy chain. The residue was either a Q or an R. During humanization studies it was determined that R at position 110 had superior properties. Additional investigation using mass spectrometry has now shown that the residue at position 110 is R. Thus, the preferred hu8G6 heavy chain version 1, 2, and 3, respectively have R at residue 110.

TABLE 1

| | | |
|---|---|---|
| | | SEQ |
| | | ID |
| Antibody | Amino Acid Sequence | NO: |

Heavy Chain CDR1 Sequences

| | | |
|---|---|---|
| 8G6 | SYTFTDYAMH | 14 |
| 1A8 | SYTFTDYTMH | 15 |
| 2B1 | GFTFSRYVMS | 16 |
| 3G9 | GFTFSRYVMS | 16 |
| 2A1 | GYDFNNDLIE | 17 |
| 2G2 | GYAFTNYLIE | 18 |

Heavy Chain CDR2 Sequences

| | | |
|---|---|---|
| 8G6 | VISTYYGNTNYNQKFKG | 19 |
| 1A8 | VIDTYYGKTNYNQKFEG | 20 |
| 2B1 | SISSG-GSTYYPDSVKG | 21 |
| 3G9 | SISSG-GRMYYPDTVKG | 22 |
| 2A1 | VINPGSGRTNYNEKFKG | 23 |
| 2G2 | VISPGSGIINYNEKFKG | 24 |

Heavy Chain CDR3 Sequences

| | | |
|---|---|---|
| 8G6 | GGLRRGDRPSLRYAMDY | 25 |
| 1A8 | GGFRRGDRPSLRYAMDS | 26 |
| 2B1 | GAIYDG- — YYVFAY | 27 |
| 3G9 | GSIYDG- — YYVFPY | 28 |
| 2A1 | IYYGPH- — SYAMDY | 29 |
| 2G2 | ID-YSG- — PYAVDD | 30 |

Light Chain CDR1 Sequences

| | | |
|---|---|---|
| 8G6 | RASQSVSTSS-YSYMY | 31 |
| 1A8 | RASQSVSIST-YSYIH | 32 |
| 2B1 | SASSSVSSS — YLY | 33 |
| 3G9 | SANSSVSSS — YLY | 34 |
| 2A1 | KASLDVRTAVA | 35 |
| 2G2 | KASQAVNTAVA | 36 |

Light Chain CDR2 Sequences

| | | |
|---|---|---|
| 8G6 | YASNLES | 37 |
| 1A8 | YASNLES | 37 |
| 2B1 | STSNLAS | 38 |
| 3G9 | STSNLAS | 38 |
| 2A1 | SASYRYT | 39 |
| 2G2 | SASYQYT | 40 |

Light Chain CDR3 Sequences

| | | |
|---|---|---|
| 8G6 | QHNWEIPFT | 41 |
| 1A8 | QHSWEIPYT | 42 |
| 2B1 | HQWSSYPPT | 43 |
| 3G9 | HQWSTYPPT | 44 |
| 2A1 | QQHYGIPWT | 45 |
| 2G2 | QHHYGVPWT | 46 |

In other related embodiments, the monoclonal antibodies used in accordance with the present invention are chimeric antibodies, i.e., those in which a cognate antibody from one species (e.g., murine, rat or rabbit) is altered by recombinant DNA technology such that part or all of the hinge and/or constant regions of the heavy and/or light chains are replaced with the corresponding components of an antibody from another species (e.g., human). Generally, the variable domains of the engineered antibody remain identical or substantially so to the variable domains of the cognate antibody. Such an engineered antibody is called a chimeric antibody and is less antigenic than the cognate antibody when administered to an individual of the species from which the hinge and/or constant region is derived (e.g., a human). Methods of making chimeric antibodies are well known in the art.

In other related embodiments, the monoclonal antibodies used in accordance with the present invention are fully human antibodies. Methods for producing such fully human monoclonal antibodies are well known in the art (see, e.g., US 2005/0255102 A1 at page 4, paragraphs 0069-0070, which are incorporated herein by reference).

In other related embodiments, the monoclonal antibodies used in accordance with the present invention are humanized versions of cognate anti-αvβ6 antibodies derived from other species. A humanized antibody is an antibody produced by recombinant DNA technology, in which some or all of the amino acids of a human immunoglobulin light or heavy chain that are not required for antigen binding (e.g., the constant regions and the framework regions of the variable domains) are used to substitute for the corresponding amino acids from the light or heavy chain of the cognate, nonhuman antibody. By way of example, a humanized version of a murine antibody to a given antigen has, on both of its heavy and light chain: (a) constant regions of a human antibody; (b) framework regions from the variable domains of a human antibody; and (c) CDRs from the murine antibody. When necessary, one or more residues in the human framework regions can be changed to residues at the corresponding positions in the murine antibody so as to preserve the binding affinity of the humanized antibody to the antigen. This change is sometimes called “back mutation.” Humanized antibodies generally are less likely to elicit an immune response in humans as compared to chimeric human antibodies because the former contain considerably fewer non-human components. Methods for producing such humanized monoclonal antibodies are well known in the art (see, e.g., US 2005/0255102 A1 at pages 4-5, paragraphs 0072-0077, which are incorporated herein by reference).

In additional such embodiments, the humanized antibodies comprise one or more CDRs in the heavy and/or light chain that are derived from the corresponding CDRs in the heavy and/or light chain of a different antibody. One suitable non-limiting example of such an antibody is a humanized 3G9 antibody comprising a light chain CDR1 that has the sequence of the light chain CDR1 derived from the 2B1 antibody (SEQ ID NO:33) instead of the sequence of the light chain CDR1 for the deposited 3G9 antibody (SEQ ID NO:34). Such a humanized 3G9 antibody having a light chain CDR1 sequence set forth in SEQ ID NO:33 is designated herein as hu3G9 (or BG00011). Another suitable non-limiting example of such an antibody is a humanized 8G6 antibody comprising a light chain CDR1 that has the sequence of the light chain CDR1 derived from the 2B1 antibody (SEQ ID NO:33) instead of the sequence of the light chain CDR1 for the deposited 8G6 antibody (SEQ ID NO: 31). Such a humanized 8G6 antibody having a light chain CDR1 sequence set forth in SEQ ID NO:33 is designated herein as hu8G9. Additional examples of such derivative antibodies, in which one or more heavy chain and/or light chain CDRs has been replaced with one or more corresponding heavy chain and/or light chain CDRs from another antibody, and which are suitable for use in accordance with the present invention, will be readily apparent to those of ordinary skill in view of the sequences depicted in Table 1 and the guidance provided herein. Suitable methods for preparing such humanized antibodies, including such derivative humanized antibodies, are familiar to those of ordinary skill and are set forth, for example, in US published application no. 2005/0255102 A1, the disclosure of which is incorporated herein by reference in its entirety.

Humanized 3G9 is a preferred antibody for use in the present methods. Design of the reshaped variable domains to produce humanized 3G9 (hu3G9) was done as follows. The 3G9 light chain variable domain corresponds to human kappa 3, and the heavy chain variable domain to human heavy subgroup 3. Three versions of each of the variable light and heavy reshaped chains were designed, as shown in Table 1A below. The first version contains the most backmutations to the murine donor sequences, while the third version contains the fewest (i.e., the most “humanized”). The CDR regions of the heavy and light chain variable domains as shown in Table 1 below are being defined by the conventional Kabat numbering classification system. However, the numbering of the sequences are represented below based on the relative linear positioning of the different sequences with respect to each other.

TABLE 1A
Heavy and Light Chain Sequences for hu3G9
Heavy Chain Sequences
(SEQ ID NO: 55-59)

| | | FR1 CDR1 FR2 |
|---|---|---|
| Murine | (1) | EVMLVESGGGLVKPGGSLKLSCAASGFTFS RYVMS WVRQTPEKRLEWVA |
| 3G9HV1 | (1) | EVMLVESGGGLVQPGGSLRLSCAASGFTFS RYVMS WVRQAPGKGLEWVA |
| 3G9HV2 | (1) | EVQLVESGGGLVQPGGSLRLSCAASGFTFS RYVMS WVRQAPGKGLEWVA |
| 3G9HV3 | (1) | EVQLVESGGGLVQPGGSLRLSCAASGFTFS RYVMS WVRQAPGKGLEWVA |
| VH3-7 | (1) | EVQLVESGGGLVQPGGSLRLSCAASGFTFS ----- WVRQAPGKGLEWVA |
| | | CDR2 FR3 |
| Murine | (50) | SISS-GGRMYYPDTVKG RFTISRDSARNILYLQMSSLRSEDTAMYYCAR |
| 3G9HV1 | (50) | SISS-GGRMYYPDTVKG RFTISRDSAKNSLYLQMNSLRAEDTAVYYCAR |
| 3G9HV2 | (50) | SISS-GGRMYYPDTVKG RFTISRDSAKNSLYLQMNSLRAEDTAVYYCAR |
| 3G9HV3 | (50) | SISS-GGRMYYPDTVKG RFTISRDNAKNSLYLQMNSLRAEDTAVYYCAR |
| VH3-7 | (50) | ----------------- RFTISRDNAKNSLYLQMNSLRAEDTAVYYCAR |

Light Chain Sequences
(SEQ ID NO: 60-66)

| | | |
|---|---|---|
| | | FR1 CDR1 FR2 |
| Murine | (1) | QIVLTQSPAIMSASPGEKVTLTC SANSSVSSSYLY WYQQKSGSSPKLWIY |
| 3G9LV1 | (1) | EIVLTQSPATLSLSPGERATLSC SASSSVSSSYLY WYQQKPGQAPRLWIY |
| 3G9LV2 | (1) | EIVLTQSPATLSLSPGERATLSC SASSSVSSSYLY WYQQKPGQAPRLWIY |
| 3G9LV3 | (1) | EIVLTQSPATLSLSPGERATLSC SASSSVSSSYLY WYQQKPGQAPRLWIY |
| 3G9LV4 | (1) | QIVLTQSPATLSLSPGERATLSC SASSSVSSSYLY WYQQKPGQAPRLWIY |
| 3G9LV5 | (1) | EIVLTQSPATLSLSPGERATLSC SASSSVSSSYLY WYQQKPGQAPRLLIY |
| L6 | (1) | EIVLTQSPATLSLSPGERATLSC -S-----------WYQQKPGQAPRLLIY |
| | | CDR2 FR3 CDR3 |
| Murine | (51) | STSNLAS GVPVRFSGSGSGTSFSLTISSMEAEDAASYFC HQWSTYPPT |
| 3G9LV1 | (51) | STSNLAS GVPVRFSGSGSGTDFTLTISSLEPEDFAVYFC HQWSTYPPT |
| 3G9LV2 | (51) | STSNLAS GVPARFSGSGSGTDFTLTISSLEPEDFAVYYC HQWSTYPPT |
| 3G9LV3 | (51) | STSNLAS GIPARFSGSGSGTDFTLTISSLEPEDFAVYYC HQWSTYPPT |
| 3G9LV4 | (51) | STSNLAS GIPARFSGSGSGTDFTLTISSLEPEDFAVYYC HQWSTYPPT |
| 3G9LV5 | (51) | STSNLAS GIPARFSGSGSGTDFTLTISSLEPEDFAVYYC HQWSTYPPT |
| L6 | (50) | ------- GIPARFSGSGSGTDFTLTISSLEPEDFAVYYC --------- |

In other preferred embodiments, the antibody used is hu3G9. The DNA and corresponding protein sequences of the different versions of hu3G9 heavy

(versions 1, 2, 3 and 5) and light (versions 1-5) variable domains are shown in Table 2 herein below. For the heavy chain variable domains, the sequences comprise:
(a) a human FR1 derived from the FR1 of VH3-7;
(b) the murine 3G9 CDR1 heavy chain sequence;
(c) a human FR2 derived from the FR2 of VH3-7;
(d) the murine 3G9 CDR2 heavy chain sequence;
(e) a human FR3 derived from the FR3 of VH3-7;
(f) the murine 3G9 CDR3 heavy chain sequence; and
(g) a human FR4 derived from a consensus framework sequence present in a large majority of human antibodies with the following sequence: WGQGTLVTVSS.

For the light chain variable domains, the sequences comprise:
(a) a human FR1 derived from the FR1 of L6;
(b) the murine 3G9 CDR1 light chain sequence with an asparagine (N) to serine (S) amino acid substitution;
(c) a human FR2 derived from the FR2 of L6;
(d) the murine 3G9 CDR2 light chain sequence;
(e) a human FR3 derived from the FR3 of L6;
(f) the murine 3G9 CDR3 light chain sequence; and
(g) a human FR4 derived from a consensus framework sequence present in a large majority of human antibodies with the following sequence: FGGGTKVEK.

TABLE 2
Heavy and Light Chain Sequences of hu3G9 Variable Domains
hu3G9 version 1 light chain
(SEQ ID NO: 47)

| | |
|---|---|
| 1 | GAGATCGTGCTGACCCAGAGCCCCGCCACCCTGAGCCTGAGCCCCGGCGAGAGGGCCACC |
| | E I V L T Q S P A T L S L S P G • R A T |
| 61 | CTGAGCTGCAGCGCCAGCAGCAGCGTGAGCAGCAGCTACCTGTACTGGTACCAGCAGAAG |
| | L S C S A S S S V S S S Y L Y W Y Q Q K |
| 121 | CCCGGCCAGGCCCCCAGGCTGTGGATCTACAGCACCAGCAACCTGGCCAGCGGCGTGCCC |
| | P G Q A P R L W I Y S T S N L A S G V P |
| 181 | GTGCGCTTCAGCGGCAGCGGCAGCGGCACCGACTTCACCCTGACCATCAGCAGCCTGGAG |
| | V R F S G S G S G T D F T L T I S S L E |
| 241 | CCCGAGGACTTCGCCGTGTACTTCTGCCACCAGTGGAGCACCTACCCCCCCACCTTCGGC |
| | P E D F A V Y F C H Q W S T Y P P T F G |
| 301 | GGCGGCACCAAGGTGGAGATCAAG |
| | G G T K V E I K |

hu3G9 version 2 light chain
(SEQ ID NO: 48)

| | |
|---|---|
| 1 | GAGATCGTGCTGACCCAGAGCCCCGCCACCCTGAGCCTGAGCCCCGGCGAGAGGGCCACC |
| | E I V L T Q S P A T L S L S P G E R A T |
| 61 | CTGAGCTGCAGCGCCAGCAGCAGCGTGAGCAGCAGCTACCTGTACTGGTACCAGCAGAAG |
| | L S C S A S S S V S S S Y L Y W Y Q Q K |
| 121 | CCCGGCCAGGCCCCCAGGCTGTGGATCTACAGCACCAGCAACCTGGCCAGCGGCGTGCCC |
| | P G Q A P R L W I Y S T S N L A S G V P |
| 181 | GCCCGCTTCAGCGGCAGCGGCAGCGGCACCGACTTCACCCTGACCATCAGCAGCCTGGAG |
| | A R F S G S G S G T D F T L T I S S L E |
| 241 | CCCGAGGACTTCGCCGTGTACTACTGCCACCAGTGGAGCACCTACCCCCCCACCTTCGGC |
| | P E D F A V Y Y C H Q W S T Y P P T F G |
| 301 | GGCGGCACCAAGGTGGAGATCAAG |
| | G G T K V E I K |

hu3G9 version 3 light chain
(SEQ ID NO: 49)

| | |
|---|---|
| 1 | GAGATCGTGCTGACCCAGAGCCCCGCCACCCTGAGCCTGAGCCCCGGCGAGAGGGCCACC |
| | E I V L T Q S P A T L S L S P G E R A T |
| 61 | CTGAGCTGCAGCGCCAGCAGCAGCGTGAGCAGCAGCTACCTGTACTGGTACCAGCAGAAG |
| | L S C S A S S S V S S S Y L Y W Y Q Q K |
| 121 | CCCGGCCAGGCCCCCAGGCTGTGGATCTACAGCACCAGCAACCTGGCCAGCGGCATCCCC |
| | P G Q A P R L W I Y S T S N L A S G I P |
| 181 | GCCCGCTTCAGCGGCAGCGGCAGCGGCACCGACTTCACCCTGACCATCAGCAGCCTGGAG |
| | A R F S G S G S G T D F T L T I S S L E |
| 241 | CCCGAGGACTTCGCCGTGTACTACTGCCACCAGTGGAGCACCTACCCCCCCACCTTCGGC |
| | P E D F A V Y Y C H Q W S T Y P P T F G |
| 301 | GGCGGCACCAAGGTGGAGATCAAG |
| | G G T K V E I K |

hu3G9 version 4 light chain
(SEQ ID NO: 50)

| | |
|---|---|
| 1 | CAGATCGTGCTGACCCAGAGCCCCGCCACCCTGAGCCTGAGCCCCGGCGAGAGGGCCACC |
| | Q I V L T Q S P A T L S L S P G E R A T |
| 61 | CTGAGCTGCAGCGCCAGCAGCAGCGTGAGCAGCAGCTACCTGTACTGGTACCAGCAGAAG |
| | L S C S A S S S V S S S Y L Y W Y Q Q K |
| 121 | CCCGGCCAGGCCCCCAGGCTGTGGATCTACAGCACCAGCAACCTGGCCAGCGGCATCCCC |
| | P G Q A P R L W I Y S T S N L A S G I P |
| 181 | GCCCGCTTCAGCGGCAGCGGCAGCGGCACCGACTTCACCCTGACCATCAGCAGCCTGGAG |
| | A R F S G S G S G T D F T L T I S S L E |
| 241 | CCCGAGGACTTCGCCGTGTACTACTGCCACCAGTGGAGCACCTACCCCCCCACCTTCGGC |
| | P E D F A V Y Y C H Q W S T Y P P T F G |
| 301 | GGCGGCACCAAGGTGGAGATCAAG |
| | G G T K V E I K |

hu3G9 version 5 light chain
(SEQ ID NO: 51)

| | |
|---|---|
| 1 | AGATCGTGCTGACCCAGAGCCCCGCCACCCTGAGCCTGAGCCCCGGCGAGAGGGCCACC |
| | E I V L T Q S P A T L S L S P G E R A T |
| 61 | TGAGCTGCAGCGCCAGCAGCAGCGTGAGCAGCAGCTACCTGTACTGGTACCAGCAGAAG |
| | L S C S A S S S V S S S Y L Y W Y Q Q K |
| 121 | CCGGCCAGGCCCCCAGGCTGCTGATCTACAGCACCAGCAACCTGGCCAGCGGCATCCCC |
| | P G Q A P R L L I Y S T S N L A S G I P |
| 181 | CCCGCTTCAGCGGCAGCGGCAGCGGCACCGACTTCACCCTGACCATCAGCAGCCTGGAG |
| | A R F S G S G S G T D F T L T I S S L E |
| 241 | CCGAGGACTTCGCCGTGTACTACTGCCACCAGTGGAGCACCTACCCCCCCACCTTCGGC |
| | P E D F A V Y Y C H Q W S T Y P P T F G |
| 301 | GGCGGCACCAAGGTGGAGATCAAG |
| | G G T K V E I K |

hu3G9 version 1 heavy chain
(SEQ ID NO: 52)

| | |
|---|---|
| 1 | AGGTGATGCTGGTGGAGAGCGGCGGCGGCCTGGTGCAGCCCGGCGGCAGCCTGAGGCTG |
| | E V M L V E S G G G L V Q P G G S L R L |
| 61 | GCTGCGCCGCCAGCGGCTTCACCTTCAGCCGCTACGTGATGAGCTGGGTGCGCCAGGCC |
| | S C A A S G F T F S R Y V M S W V R Q A |
| 121 | CCGGCAAGGGCCTGGAGTGGGTGGCCAGCATCAGCAGCGGAGGCCGCATGTACTACCCC |
| | P G K G L E W V A S I S S G G R M Y Y P |
| 181 | ACACCGTGAAGGGCCGCTTCACCATCAGCCGCGACAGCGCCAAGAACAGCCTGTACCTG |
| | D T V K G R F T I S R D S A K S L Y Y L |
| 241 | AGATGAACAGCCTGCGCGCCGAGGACACCGCCGTGTACTACTGCGCCCGCGGCAGCATC |
| | Q M N S L R A E D T A V Y Y C A R G S I |
| 301 | ACGACGGCTACTACGTGTTCCCCTACTGGGGCCAGGGCACCCTGGTGACCGTGAGCTCC |
| | Y D G Y Y V F P Y W G Q G T L V T V S S |

hu3G9 version 2 heavy chain
(SEQ ID NO: 53)

| | |
|---|---|
| 1 | AGGTGCAGCTGGTGGAGAGCGGCGGCGGCCTGGTGCAGCCCGGCGGCAGCCTGAGGCTG |
| | E V Q L V E S G G G L V Q P G G S L R L |
| 61 | GCTGCGCCGCCAGCGGCTTCACCTTCAGCCGCTACGTGATGAGCTGGGTGCGCCAGGCC |
| | S C A A S G F T F S R Y V M S W V R Q A |
| 121 | CCGGCAAGGGCCTGGAGTGGGTGGCCAGCATCAGCAGCGGAGGCCGCATGTACTACCCC |
| | P G K G L E W V A S I S S G G R M Y Y P |
| 181 | ACACCGTGAAGGGCCGCTTCACCATCAGCCGCGACAGCGCCAAGAACAGCCTGTACCTG |
| | D T V K G R F T I S R D S A K N S L Y L |
| 241 | AGATGAACAGCCTGCGCGCCGAGGACACCGCCGTGTACTACTGCGCCCGCGGCAGCATC |
| | Q M N S L R A E D T A V Y Y C A R G S I |
| 301 | ACGACGGCTACTACGTGTTCCCCTACTGGGGCCAGGGCACCCTGGTGACCGTGAGCTCC |
| | Y D G Y Y V F P Y W G Q G T L V T V S S |

hu3G9 versions 3 and 5 heavy chain

| | |
|---|---|
| | (SEO ID NO: 54) |
| 1 | AGGTGCAGCTGGTGGAGAGCGGCGGCGGCCTGGTGCAGCCCGGCGGCAGCCTGAGGCTG |
| | E V Q L V E S G G G L V Q P G G S L R L |
| 61 | GCTGCGCCGCCAGCGGCTTCACCTTCAGCCGCTACGTGATGAGCTGGGTGCGCCAGGCC |
| | S C A A S G F T F S R Y V M S W V R Q A |
| 121 | CCGGCAAGGGCCTGGAGTGGGTGGCCAGCATCAGCAGCGGAGGCCGCATGTACTACCCC |
| | P G K G L E W V A S I S S G G R M Y Y P |
| 181 | ACACCGTGAAGGGCCGCTTCACCATCAGCCGCGACAACGCCAAGAACAGCCTGTACCTG |
| | D T V K G R F T I S R D N A K S L Y Y L |
| 241 | AGATGAACAGCCTGCGCGCCGAGGACACCGCCGTGTACTACTGCGCCCGCGGCAGCATC |
| | Q M N S L R A E D T A V Y Y C A R G S I |
| 301 | ACGACGGCTACTACGTGTTCCCCTACTGGGGCCAGGGCACCCTGGTGACCGTGAGCTCC |
| | Y D G Y Y V F P Y W G Q G T L V T V S S |

In additional embodiments, three versions of the 8G6 variable light reshaped chain and three versions of the 8G6 variable heavy reshaped chain may be used as preferred antibodies in the present invention. The first version contains the most backmutations and the third version contain the fewest (i.e., is the most humanized). Table 3 below displays the heavy and light chain variable domain sequences for humanized 8G6 (hu8G6) antibodies.

TABLE 3
Heavy and Light Chain Sequences for hu8G6
Table 3a-Heavy Chain Sequences
(SEQ ID NO: 67-71)

| | | FR1 CDR1 FR2 |
|---|---|---|
| Murine | (1) | QVQLQQSGPELVRPGVSVKISCKGSSYTFT DYAMH WVKLSHAKSLEWIG |
| 8G6HV1 | (1) | QVQLVQSGAEVKKPGASVKVSCKGSSYTFT DYAMH WVRLAPGQGLEWIG |
| 8G6HV2 | (1) | QVQLVQSGAEVKKPGASVKVSCKASGYTFT DYAMH WVRQAPGQGLEWIG |
| 8G6HV3 | (1) | QVQLVQSGAEVKKPGASVKVSCKASGYTFT DYAMH WVRQAPGQGLEWMG |
| VH1-2 | (1) | QVQLVQSGAEVKKPGASVKVSCKASGYTFT ----- WVRQAPGQGLEWMG |
| | | CDR2 FR3 |
| Murine | (50) | VISTYYGNTNYNQKFKG KATMTVDKSSSTAYMELARLTSEDSAVYYCAR |
| 8G6HV1 | (50) | VISTYYGNTNYNQKFKG RATMTVDKSISTAYMELSRLRSDDTAVYYCAR |
| 8G6HV2 | (50) | VISTYYGNTNYNQKFKG RATMTVDKSISTAYMELSRLRSDDTAVYYCAR |
| 8G6HV3 | (50) | VISTYYGNTNYNQKFKG RATMTVDKSISTAYMELSRLRSDDTAVYYCAR |
| VH1-2 | (50) | ------------------RVTMTRDTSISTAYMELSRLRSDDTAVYYCAR |

Table 3b-Light Chain Sequences
(SEQ ID NO: 72-76)

| | | |
|---|---|---|
| | | FR1 CDR1 FR2 |
| Mu | (1) | DIVLTQSPASLAVSLGQRAIISC RASQSVSTSSYSYMY WYQQKPGQSPKFLIK |
| 8G6LV1 | (1) | DIVLTQSPATLSLSPGERATLSC RASQSVSTSSYSYMY WYQQKPGQAPRFLIK |
| 8G6LV2 | (1) | EIVLTQSPATLSLSPGERATLSC RASQSVSTSSYSYMY WYQQKPGQAPRFLIK |
| 8G6LV3 | (1) | EIVLTQSPATLSLSPGERATLSC RASQSVSTSSYSYMY WYQQKPGQAPRLLIK |
| L6 | (1) | EIVLTQSPATLSLSPGERATLSC --------------- WYQQKPGQAPRLLIY |
| | | CDR2 FR3 CDR3 |
| Murine | (54) | YASNLES GVPARFSGSGSGTDFTLNIHPVEEEDTATYYC QHNWEIP |
| 8G6LV1 | (54) | YASNLES GIPARFSGSGSGTDFTLTISSLEPEDFAVYYC QHNWEIP |
| 8G6LV2 | (54) | YASNLES GIPARFSGSGSGTDFTLTISSLEPEDFAVYYC QHNWEIP |
| 8G6LV3 | (54) | YASNLES GIPARFSGSGSGTDFTLTISSL•PEDFAVYYC QHNWEIP |
| L6 | (50) | ------- GIPARFSGSGSGTDFTLTISSLEPEDFAVYYC ------ |

The protein sequences of the different versions of hu8G6 heavy (versions 1, 2 and 3) and light (versions 1, 2 and 3) variable domains are shown in Table 4. For the heavy chain variable domains, the sequences comprise:
(a) a human FR1 derived from the FR1 of VH1-2;
(b) the murine 8G6 CDR1 heavy chain sequence;
(c) a human FR2 derived from the FR2 of VH1-2;
(d) the murine 8G6 CDR2 heavy chain sequence;
(e) a human FR3 derived from the FR3 of VH1-2;
(f) the murine 8G6 CDR3 heavy chain sequence; and
(g) a human FR4 derived from a consensus framework sequence which is 100% identical to the human framework gi|392715 from the NR database and is present in a large majority of human antibodies with the following sequence: WGQGTL VTVSS.

For the light chain variable domains, the sequences comprise:
(a) a human FR1 derived from the FR1 of L6;
(b) the murine 8G6 CDR1 light chain sequence;
(c) a human FR2 derived from the FR2 of L6;
(d) the murine 8G6 CDR2 light chain sequence;
(e) a human FR3 derived from the FR3 of L6;
(f) the murine 8G6 CDR3 light chain sequence; and
(g) a human FR4 derived from a consensus framework sequence present in a large majority of human antibodies with the following sequence: FGGGTKVEIK

TABLE 4
Heavy and Light Chain Sequences of
hu8G6 Variable Domains

hu8G6 version 1 heavy chain
(SEQ ID NO: 11)
QVQLVQSGAEVKKPGASVKVSCKGSSYTFTDYAMHWVRLAPGQGLE
WIGVISTYYGNTNYNQKFKGRATMTVDKSISTAYMELSRLRSDDTAV
YYCARGGLRRGDRPSLRYAMDYWGQGTLVTVSS
hu8G6 version 2 heavy chain
(SEQ ID NO: 12)
QVQLVQSGAEVKKPGASVKVSCKASGYTFTDYAMHWVRQAPGQGL
EWIGVISTYYGNTNYNQKFKGRATMTVDKSISTAYMELSRLRSDDTA
VYYCARGGLRRGDRPSLRYAMDYWGQGTLVTVSS
hu8G6 version 3 heavy chain
(SEQ ID NO: 13)
QVQLVQSGAEVKKPGASVKVSCKASGYTFTDYAMHWVRQAPGQGL
EWMGVISTYYGNTNYNQKFKGRATMTVDKSISTAYMELSRLRSDDTA
VYYCARGGLRRGDRPSLRYAMDYWGQGTLVTVSS
hu8G6 version 1 light chain
(SEQ ID NO: 8)
DIVLTQSPATLSLSPGERATLSCRASQSVSTSSYSYMYWYQQKPGQAP
RFLIICYASNLESGFP ARFSGSGSGTDFTLTISSLEPEDFAVYYCQHNW
EiPFTFGGGTKVEIK
hu8G6 version 2 light chain
(SEQ ID NO: 9)
EIVLTQSPATLSLSPGERATLSCRASQSVSTSSYSYMYWYQQKPGQAP
RFLIKYASNLESGIPARFSGSGSGTDFTLTISSLEPEDFAVYYCQHNWEI
PFTFGGGTKVEIK
hu8G6 version 3 light chain
(SEQ ID NO: 10)
EIVLTQSPATLSLSPGERATLSCRASQSVSTSSYSYMYWYQQKPGQAP
RLLIKYASNLESGIP ARFSGSGSGTDFTLTISSLEPEDFAVYYCQHNWE
IPFTFGGGTKVEIK

Additional sequences that may be used herein include for example, those for pKJS195 vector-3G9 version 5 light chain (SEQ ID No:77); pKJS189 vector-3G9 vector 3 heavy chain (SEQ ID NO:78); pKJS196 vector-aglycosyl-3G9 version 3 heavy chain (SEQ ID NO:79); hu3G9 version 1 light chain (SEQ ID NO:80); hu3G9 version 2 light chain (SEQ ID NO:81); hu3G9 version 3 light chain (SEQ ID NO:82); hu3G9 version 4 light chain (SEQ ID NO:83); hu3G9 version 5 light chain (SEQ ID NO:84); hu3G9 version 1 heavy chain (SEQ ID NO:85); hu3G9 version 2 heavy chain (SEQ ID NO:86); hu3G9 versions 3 and 5 heavy chain (SEQ ID NO:87); human FR4 derived from a consensus framework sequence (SEQ ID NO:88); human FR4 derived from a consensus framework sequence SEQ ID NO:89).

The following describes the backmutations in the reshaped variable light chain: include:

E1D—This has been shown to influence CDR conformation/antigen binding (Kolbinger et al., Protein Eng., 8:971-980 (1993)). In the model, it might interact with the backbone or sidechains of S26, Q27 and/or E93 in CDRs L1 and L3. It is removed in versions 2 and 3 since the substitution is conservative.

L46F—This is a VH/VL packing interface residue. It also appears to be right underneath CDR-L2 residue E55. It is removed in version 3.

Y49K—This is adjacent to CDR-L2 and appears to be interacting with residue E55 in the model. This is likely to be a very important backmutation and, therefore, is not removed.

The following describes the backmutations in the reshaped variable heavy chain:

A24G—This is a canonical residue for CDR-H1.

Conservative mutation. Removed in version 2:

G26S—This is canonical residue for CDR-H1.

Conservative mutation. Removed in version 2.

Q39L—This is packing interface residue. It has very limited interaction with the light chain and, therefore, is removed in version 2. M481-This is a common backmutation. In the model it may be interacting with Y59 and F63 in CDR-H2. It is dismissed in version 3. V68A—This residues is located underneath CDR-H2 possibly interacting with Y59 and F63.

R72V—This is a canonical residue for CDR-H2.

T74K—This residue is located underneath CDR-H2 possibly interacting with Y53 or contacting antigen directly.

In other embodiments of the invention, antagonists of αᵥβ₆ are used which are peptides, polypeptides, proteins, or peptidomimetics designed as ligands for αᵥβ₆ on the basis of the presence of the cell adhesion domain arginine-glycine-aspartic acid (RGD). The design of such molecules as ligands for the integrins is exemplified, for example, in Pierschbacher et al., J. Cell. Biochem. 56:150-154 (1994)); Ruoslahti, Ann Rev. Cell. Dev. Biol. 12:697-715 (1996); Chorev et al. Biopolymers 37:367-375 (1995)); Pasqualini et al., J. Cell. Biol. 130:1189-1196 (1995)); and Smith et al., J. Biol, Chem, 269:32788-32795 (1994)).

In some embodiments of the invention, antisense nucleic acid molecules are used as antagonists of αᵥβ₆. Antisense nucleic acid molecules are complementary oligonucleotide strands of nucleic acids designed to bind to a specific sequence of nucleotides to inhibit production of a targeted protein. The nucleotide sequence of the β6 integrin subunit was disclosed in U.S. Pat. No. 5,962,643, incorporated herein by reference in its entirety. These agents may be used alone or in combination with other αᵥβ₆ antagonists, such as those described herein. The antisense antagonist may be provided as an antisense oligonucleotide such as RNA (see, for example, Murayama et al. Antisense Nucleic Acid Drug Dev. 7:109-114 (1997)). Antisense genes may also be provided in a viral vector, such as, for example, in hepatitis B virus (see, for example, Ji et al., J. Viral Hepat. 4:167-173 (1997)); in adeno-associated virus (see, for example, Xiao et al. Brain Res. 756:76-83 (1997)); or in other systems including but not limited to an HVJ(Sendai virus)-liposome gene delivery system (see, for example, Kaneda et al. Ann, N.Y. Acad. Sci. 811:299-308 (1997)); a “peptide vector” (see, for example, Vidal et al. CR Acad. Sci III 32:279-287 (1997)); as a gene in an episomal or plasmid vector (see, for example, Cooper et al. Proc. Natl. Acad. Sci. U.S.A. 94:6450-6455 (1997), Yew et al. Hum Gene Ther 8:575-584 (1997)); as a gene in a peptide-DNA aggregate (see, for example, Niidome et al. J. Biol. Chem. 272:15307-15312 (1997)); as “naked DNA” (see, for example, U.S. Pat. Nos. 5,580,859 and 5,589,466); and in lipidic vector systems (see, for example, Lee et al. Crit Rev Ther Drug Carrier Syst, 14.173-206 (1997)).

In some embodiments of the invention, antagonists are used which are peptides, polypeptides, proteins, or peptidomimetics designed as ligands for αᵥβ₆ on the basis of the presence of the cell adhesion domain arginine-glycine-aspartic acid (RGD). The design of such molecules as ligands for the integrins is exemplified, for example, in Pierschbacher et al., J. Cell. Biochem. 56:150-154 (1994); Ruoslahti, Ann Rev. Cell. Dev. Biol. 12:697-715 (1996); Chorev et al. Biopolymers 37:367-375 (1995); Pasqualini et al., J. Cell. Biol. 130:1189-1196 (1995); and Smith et al., J. Biol, Chem, 269:32788-32795 (1994).

Candidate antagonists of αᵥβ₆ can be screened for function by a variety of techniques known in the art and/or disclosed within the instant application, such as protection against bleomycin-induced fibrosis in a mouse model (WO03/100033, incorporated herein by reference in its entirety); inhibition of the proliferation of tumor cells (Agrez et al., J. Cell Bio., 127-547-556 (1994)); and inhibition of cell migration and/or inhibition of cell adhesion.

Conjugates and Other Modifications of αᵥβ₆-Binding Ligands

In certain embodiments, the ligands, e.g., the antibodies, that bind to or otherwise antagonize αᵥβ₆ can be used in unconjugated form. In other embodiments, the ligands, e.g., the antibodies, that bind to or otherwise antagonize αᵥβ₆ can be conjugated, e.g., to a detectable label, a drug, a prodrug or an isotope. The humanized antibodies may comprise a moiety (e.g., biotin, fluorescent moieties, radioactive moieties, histidine tag or other peptide tags) for easy isolation or detection. The humanized antibodies may also comprise a moiety that can prolong their serum half life, for example, a polyethylene glycol (PEG) moiety or a (poly)sialic acid moiety, an FMOC moiety or other chemical modification commonly used to prolong half life of a protein in circulation.

In certain methods of the invention described in more detail below, such as methods of detecting αᵥβ₆ expression in cells or tissues as a measure of the potential of epithelial cells to be responsive to αᵥβ₆-binding ligands, the αᵥβ₆-binding ligands (e.g., antibodies) are conjugated to one or more detectable labels. For such uses, the αᵥβ₆-binding ligands, e.g., αᵥβ₆-binding antibodies, may be detectably labeled by covalent or non-covalent attachment of a chromogenic, enzymatic, radioisotopic, isotopic, fluorescent, toxic, chemiluminescent, nuclear magnetic resonance contrast agent or other label.

Examples of suitable chromogenic labels include diaminobenzidine and 4-hydroxyazo-benzene-2-carboxylic acid.

Examples of suitable enzyme labels include malate dehydrogenase, staphylococcal nuclease, Δ-5-steroid isomerase, yeast-alcohol dehydrogenase, α-glycerol phosphate dehydrogenase, triose phosphate isomerase, peroxidase, alkaline phosphatase, asparaginase, glucose oxidase, β-galactosidase, ribonuclease, urease, catalase, glucose-6-phosphate dehydrogenase, glucoamylase, and acetylcholine esterase.

Examples of suitable radioisotopic labels include ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ¹⁰⁹Pd, etc. ¹¹¹In is a preferred isotope where in vivo imaging is used since its avoids the problem of dehalogenation of the ¹²⁵I or ¹³¹I-labeled αᵥβ₆-binding ligands by the liver. In addition, this radionucleotide has a more favorable gamma emission energy for imaging (Perkins et al., Eur. J. Nucl. Med. 10:296-301 (1985); Carasquillo et al., J. Nucl. Med. 28:281-287 (1987)). For example, ¹¹¹In coupled to monoclonal antibodies with 1-(P-isothiocyanatobenzyl)-DPTA has shown little uptake in non-tumorous tissues, particularly the liver, and therefore enhances specificity of tumor localization (Esteban et al., J. Nucl. Med. 28:861-870 (1987)). Examples of suitable non-radioactive isotopic labels include ¹⁵⁷Gd, ⁵⁵Mn, ¹⁶²Dy, ⁵²Tr, and ⁵⁶Fe.

Examples of suitable fluorescent labels include an ¹⁵²Eu label, a fluorescein label, an isothiocyanate label, a rhodamine label, a phycoerythrin label, a phycocyanin label, an allophycocyanin label, a Green Fluorescent Protein (GFP) label, an o-phthaldehyde label, and a fluorescamine label.

Examples of chemiluminescent labels include a luminol label, an isoluminol label, an aromatic acridinium ester label, an imidazole label, an acridinium salt label, an oxalate ester label, a luciferin label, a luciferase label, and an aequorin label.

Examples of nuclear magnetic resonance contrasting agents include heavy metal nuclei such as Gd, Mn, and iron.

Typical techniques for binding the above-described labels to αᵥβ₆-binding ligands, e.g., αᵥβ₆-binding antibodies, are provided by Kennedy et al., Clin. Chim. Acta 70:1-31 (1976), and Schurs et al., Clin. Chim. Acta 81:1-40 (1977). Coupling techniques mentioned in the latter are the glutaraldehyde method, the periodate method, the dimaleimide method, the m-maleimidobenzyl-N-hydroxy-succinimide ester method, all of which methods are incorporated by reference herein.

Alternatively, the αᵥβ₆-binding ligand can be conjugated to one or more calicheamicin molecules. The calicheamicin family of antibiotics are capable of producing double-stranded DNA breaks at sub-picomolar concentrations. Structural analogues of calicheamicin which may be used include, but are not limited to, γ₁^{I}, α₂^{I}, α₃^{I}, N-acetyl-γ₁^{I}, PSAG and Φ₁^{I} (Hinman et al. Cancer Research 53: 3336-3342 (1993) and Lode et al. Cancer Research 58: 2925-2928 (1998)).

A variety of radioactive isotopes are also available for the production of radioconjugated αᵥβ₆-binding ligands for use in therapeutic methods of the invention. Examples include ²¹¹At, ¹³¹I, ¹²⁵I, ⁹⁰Y, ¹⁸⁶Re, ¹⁸⁸Re, ¹⁵³Sm, ²¹²Bi, ³²P and radioactive isotopes of Lu.

In yet another embodiment, the αᵥβ₆-binding ligand may be conjugated to a “receptor” (such streptavidin) for utilization in “pretargeting” wherein the αᵥβ₆-binding ligand-receptor conjugate is administered to the patient, followed by removal of unbound conjugate from the circulation using a clearing agent and then administration of a “ligand” (e.g. avidin) which is conjugated to a cytotoxic agent (e.g., a radionucleotide).

The αᵥβ₆-binding ligands of the present invention may also be conjugated with a prodrug-activating enzyme which converts a prodrug (e.g. a peptidyl chemotherapeutic agent, see WO 81/01145) to an active drug. See, for example, WO 88/07378 and U.S. Pat. No. 4,975,278. The enzyme component of such conjugates includes any enzyme capable of acting on a prodrug in such a way so as to covert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as Serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as O-galactosidase and neuraminidase useful for converting glycosylated prodrugs into free drugs; P-lactamase useful for converting drugs derivatized with P-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs.

Enzymes can be covalently bound to the αᵥβ₆-binding ligand by techniques well known in the art such as the use of the heterobifunctional crosslinking reagents. Alternatively, fusion proteins comprising at least the antigen binding region of a αᵥβ₆-binding ligand of the invention linked to at least a functionally active portion of an enzyme can be constructed using recombinant DNA techniques well known in the art (see, e.g., Neuberger et al., Nature 312: 604-608 (1984)).

A variety of therapeutic agents can be coupled to the targeting humanized antibody. Preferably, a humanized antibody that internalizes upon binding would be best, however, the use of non-internalizing humanized antibodies is not precluded. The list of asthma-treating drugs one could use for preparing conjugates is extensive and one of skill in the art would know how to make chemical modifications to the desired compound in order to make reactions of that compound more convenient for purposes of preparing conjugates of the invention. For example, the drug would be coupled via “releasable linkers that are differentially more stable in serum yet release the active drug inside the tumor cell. Several release mechanisms could be used, depending on the specific drug. Examples of these release mechanism include the use of acid-sensitive hydrazones, redox sensitive linkers, e.g., disulfide, and proteolytically-cleaved peptide linkers.

Any of the above antibody conjugates also includes the use of fragments Fab, F(ab′)25 scFvs, minibodies, CH2 domain-deleted antibody constructs, and FcRn- mutants. These Ab fragments or generically-modified constructs have different pharmacokinetic, tumor penetration, and tumor localization properties from intact IgG that may afford advantages in particular applications. For example, the faster-clearing Fab may be useful for diagnostics applications for radioimmunodiagnostic applications. On the other hand, for radioimmunotherapy or drug targeting, selecting a targeting vehicle with a longer serum tm may be more effective.

### Therapeutic Methods

In certain embodiments of the invention, the methods of the present invention can be used therapeutically in regimens for treating mammals afflicted with certain diseases, particularly with certain symptoms of asthma as disclosed herein. Such methods of the invention are useful in treating and/or preventing asthma and associated symptoms. Particularly amenable to such an approach are those tissues or cells that are protected from the increase in airway sensitivity seen when challenged by an allergen by reducing or blocking the expression of the integrin αᵥβ₆. Methods according to this aspect of the invention comprise, for example, (a) identifying a patient with asthma or asthma-related symptoms, and (b) treating the patient with one or more αᵥβ₆-binding ligands, such as one or more αᵥβ₆-binding antibodies or fragments thereof. Methods according to this aspect of the invention further comprise, for example, (a) identifying a patient with increased susceptibility to asthma or asthma-related symptoms, and (b) treating the patient with one or more αᵥβ₆-binding ligands, such as one or more aᵥβ₆-binding antibodies or fragments thereof.

Preferred mammals for treatment include monkeys, apes, cats, dogs, cows, pigs, horses, rabbits and humans. Particularly preferred are humans.

In related embodiments, as described above, the invention provides methods of reducing or preventing asthma in a patient, comprising administering to the patient a therapeutically effective amount of one or more ligands that binds to one or more subunits of integrin αᵥβ₆ on one or more cells in the airway epithelia, wherein the binding of the ligand to the integrin results in the protection, reduction or prevention of an allergen-induced increase in airway mast cells.

In such therapeutic methods of the invention, the αᵥβ₆-binding ligand or fragments thereof may be administered to the subject or patient by any suitable means, including parenteral, intrapulmonary, intracranial, transdermal and intranasal. Parenteral infusions include intramuscular, intravenous, intraarterial, intraperitoneal, or subcutaneous administration. In addition, the αᵥβ₆-binding ligand or fragments thereof may suitably be administered by pulse infusion, e.g., with declining doses of the αᵥβ₆-binding ligand or fragments thereof. Preferably the dosing is given by injections, most preferably intravenous or subcutaneous injections, depending in part on whether the administration is brief or chronic.

In some embodiments, the αᵥβ₆-binding ligand or fragments thereof may be administered to the subject or patient by aerosol. For aerosol administration, the compositions of the invention are preferably supplied in finely divided form along with a surfactant and propellant. Typical percentages of compositions of the invention are 0.01%-20% by weight, preferably 1-10%. The surfactant must, of course, be nontoxic, and preferably soluble in the propellant. Representative of such agents are the esters or partial esters of fatty acids containing from 6 to 22 carbon atoms, such as c-aproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

In carrying out these therapeutic methods of the invention, αᵥβ₆-binding ligands, such as αᵥβ₆-binding antibodies or fragments thereof, or other αᵥβ₆ antagonists, may be administered to patients in the form of therapeutic formulations (which are also referred to herein interchangeably and equivalently as pharmaceutical compositions). Therapeutic formulations of the αᵥβ₆-binding ligands or fragments thereof used in accordance with the present invention are prepared for storage by mixing a αᵥβ₆-binding ligand or fragment thereof having the desired degree of purity with optional pharmaceutically acceptable carriers, excipients or stabilizers (Remington's Pharmaceutical Sciences 16th edition, Osol, A. Ed. (1980)), for example in the form of lyophilized formulations or aqueous solutions. In addition to the pharmacologically active compounds such as the αᵥβ₆-binding ligands or fragments thereof, the compositions used in the therapeutic methods of the invention can contain one or more suitable pharmaceutically acceptable carriers comprising excipients and auxiliaries that facilitate processing of the active compounds into preparations that can be used pharmaceutically. The pharmaceutical preparations of the present invention are manufactured in a manner that is, itself, known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example, lactose or sucrose, mannitol or sorbitol, cellulose preparations and/or calcium phosphates, for example, tricalcium phosphate or calcium hydrogen phosphate, as well as binders, such as starch paste, using, for example, maize starch, wheat starch, rice starch, potato starch, gelatin, tragacanth, methyl cellulose, hydroxypropylmethylcellulose, sodium carboxy-methylcellulose, and/or polyvinyl pyrrolidone. If desired, disintegrating agents can be added, such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example silica, talc, stearic acid or salts thereof, such as magnesium stearate or calcium stearate, and/or polyethylene glycol. Dragee cores are provided with suitable coatings, that, if desired, are resistant to gastric juices. For this purpose, concentrated saccharide solutions can be used, which may optionally contain gum arabic, talc, polyvinyl pyrrolidone, polyethylene glycol, and/or titanium dioxide, lacquer solutions and suitable organic solvents or solvent mixtures. In order to produce coatings resistant to gastric juices, solutions of suitable cellulose preparations, such as acetylcellulose phthalate or hydroxypropylmethylcellulose phthalate, are used. Dye stuffs or pigments can be added to the tablets or dragee coatings, for example, for identification or in order to characterize combinations of active compound doses.

Other pharmaceutical preparations that can be used orally include push-fit capsules made of gelatin, as well as soft, sealed capsules made of gelatin and a plasticizer such as glycerol or sorbitol. The push-fit capsules can contain the active compounds in the form of granules that may be mixed with fillers such as lactose, binders such as starches, and/or lubricants such as talc or magnesium stearate and, optionally, stabilizers. In soft capsules, the active compounds are preferably dissolved or suspended in suitable liquids such as fatty oils or liquid paraffin. In addition, stabilizers may be added.

Suitable formulations for parenteral administration include aqueous solutions of the active compounds in water-soluble form, for example water-soluble salts and alkaline solutions. Alkaline salts can include ammonium salts prepared, for example, with Tris, choline hydroxide, bis-Tris propane, N-methylglucamine, or arginine. In addition, suspensions of the active compounds as appropriate oily injection suspensions can be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400). Aqueous injection suspensions can contain substances that increase the viscosity of the suspension, for example sodium carboxymethyl cellulose, sorbitol, and/or dextran. Optionally, the suspension may also contain stabilizers.

The compounds of the present invention may be administered to the eye in animals and humans as a drop, or within ointments, gels, liposomes, or biocompatible polymer discs, pellets or carried within contact lenses. The intraocular composition may also contain a physiologically compatible ophthalmic vehicle as those skilled in the art can select using conventional criteria. The vehicles may be selected from the known ophthalmic vehicles which include but are not limited to water, polyethers such as polyethylene glycol 400, polyvinyls such as polyvinyl alcohol, povidone, cellulose derivatives such as carboxymethylcellulose, methylcellulose and hydroxypropyl methylcellulose, petroleum derivatives such as mineral oil and white petrolatum, animal fats such as lanolin, vegetable fats such as peanut oil, polymers of acrylic acid such as carboxylpolymethylene gel, polysaccharides such as dextrans and glycosaminoglycans such as sodium chloride and potassium, chloride, zinc chloride and buffer such as sodium bicarbonate or sodium lactate. High molecular weight molecules can also be used. Physiologically compatible preservatives which do not inactivate the compounds of the present invention in the composition include alcohols such as chlorobutanol, benzalkonium chloride and EDTA, or any other appropriate preservative known to those skilled in the art.

Lyophilized formulations of antibodies adapted for subcutaneous administration are described in U.S. Pat. No. 6,267,958, the disclosure of which is incorporated herein by reference in its entirety. Such lyophilized formulations may be reconstituted with a suitable diluent to a high protein concentration and the reconstituted formulation may be administered subcutaneously to the patient to be treated herein.

The αᵥβ₆-binding ligands may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations of αᵥβ₆-binding ligands may be prepared. Suitable examples of sustained-release preparations include semipermeable matrices of solid hydrophobic polymers containing the αᵥβ₆-binding ligand, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

In certain exemplary embodiments of the invention, the αᵥβ₆-binding ligands or fragments thereof are administered to the patient (e.g., intravenously) in a dosage of between about 1 mg/m² and about 500 mg/m². For instance, the αᵥβ₆-binding ligand or fragments thereof may be administered in a dosage of about 1 mg/m², 2 mg/m², 3 mg/m², 4 mg/m², 5 mg/m², 10 mg/m², 15 mg/m², 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m², 125 mg/m², 130 mg/m², 135 mg/m², 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², 200 mg/m², 205 mg/m², 210 mg/m², 215 mg/m², 220 mg/m², 225 mg/m², 230 mg/m², 235 mg/m², 240 mg/m², 245 mg/m², 250 mg/m², 255 mg/m², 260 mg/m², 265 mg/m², 270 mg/m², 275 mg/m², 280 mg/m², 285 mg/m², 290 mg/m², 295 mg/m², 300 mg/m², 305 mg/m², 310 mg/m², 315 mg/m², 320 mg/m², 325 mg/m², 330 mg/m², 335 mg/m², 340 mg/m², 345 mg/m², 350 mg/m², 355 mg/m², 360 mg/m², 365 mg/m², 370 mg/m², 375 mg/m², 380 mg/m², 385 mg/m², 390 mg/m², 395 mg/m² or 400 mg/m².

The αᵥβ₆-binding ligand or fragments thereof can be administered according to a wide variety of dosing schedules. For example, the αᵥβ₆-binding ligand or fragments thereof can be administered once daily for a predetermined amount of time (e.g., four to eight weeks, or more), or according to a weekly schedule (e.g., one day per week, two days per week, three days per week, four days per week, five days per week, six days per week or seven days per week) for a predetermined amount of time (e.g., four to eight weeks, or more). A specific example of a “once weekly” dosing schedule is administration of the αᵥβ₆-binding ligand or fragments thereof on days 1, 8, 15 and 22 of the treatment period. In alternative embodiments the αᵥβ₆-binding ligand fragments thereof may be administered intermittently over a period of months. For example, the αᵥβ₆-binding ligand or fragments thereof may be administered weekly for three consecutive weeks biannually (i.e., repeat the weekly dosing schedule every six months). It will be appreciated that such administration regimens may be continued for extended periods (on the order of years) to maintain beneficial therapeutic effects provided by initial treatments. In yet other embodiments such maintenance therapy may be effected following an acute dosing regimen designed to reduce the immediate symptoms of the cancerous, metastatic or in situ carcinoma condition.

The amount of αᵥβ₆-binding ligand or fragments thereof administered each time throughout the treatment period can be the same; alternatively, the amount administered each time during the treatment period can vary (e.g., the amount administered at a given time can be more or less than the amount administered previously). For example, doses given during maintenance therapy may be lower than those administered during the acute phase of treatment. Appropriate dosing schedules depending on the specific circumstances will be apparent to persons of ordinary skill in the art.

In certain embodiments of the invention, multiple types or species of αᵥβ₆-binding ligands are combined with one another and administered to a patient to treat asthma or asthma related conditions. For example, the invention contemplates the administration of two or more different αᵥβ₆-binding antibodies or fragments thereof to a patient, such as those disclosed herein. When multiple αᵥβ₆-binding ligands or fragments thereof are administered to a patient, the different αᵥβ₆-binding ligands and/or TGF-β-blocking agents or fragments thereof can be administered together in a single pharmaceutical composition, or, more preferably, can be administered sequentially in separate dosages. The effective amount of such other agents depends on the amount of αᵥβ₆-binding ligand or fragments thereof present in the formulation, the type of disease or disorder or treatment, and other factors.

The present invention also includes methods for treating asthma conditions that comprise administering to a patient a first agent in conjunction with a second agent, wherein the first agent is a αᵥβ₆-binding ligand and the second agent is an agent that is useful for treating asthma or in situ asthma conditions but that is not necessarily a αᵥβ₆-binding ligand. By administering a first agent “in conjunction with” a second agent is meant that the first agent can be administered to the patient prior to, simultaneously with, or after, administering the second agent to the patient, such that both agents are administered to the patient during the therapeutic regimen. For example, according to certain such embodiments of the invention, a αᵥβ₆-binding ligand is administered to a patient in conjunction (i.e., before, simultaneously with, or after) administration of an antagonist of one or more other integrin receptors (e.g., α₁β₁, α₄β₁, αᵥβ₈, αᵥβ₅, α₅β₁, etc.) to the patient, including antibodies, polypeptide antagonists and/or small molecule antagonists specific for one or more integrin receptors (e.g., α₁β₁, α₄β₁, αᵥβ₈, αᵥβ₅, α₅β₁, etc.) which are known in the art.

In certain embodiments of this aspect of the invention, the second agent that is administered in conjunction with an αᵥβ₆-binding ligand or fragments thereof is, e.g., a steroid, a cytotoxic compound (including those described elsewhere herein, and particularly paclitaxel, gemicitabine or adriamycin (doxorubicin), a radioisotope (including those described elsewhere herein), a prodrug-activating enzyme (including those described elsewhere herein), colchicine, oxygen, an antioxidant (e.g., N-acetylcysteine), a metal chelator (e.g., terathiomolybdate), IFN-β, IFN-γ, alpha-antitrypsin and the like. Additional second agents or compounds that can be administered to a patient in conjunction with one or more first agents, such as one or more αᵥβ₆-binding ligands, for therapeutic purposes according to this aspect of the invention, will be familiar to those of ordinary skill in the art; the use of such additional second agents or compounds is therefore considered to be encompassed by the present invention.

### Symptoms and Related Conditions

In additional embodiments, the present invention is directed to methods of treating a mammal having or at risk of having symptoms of asthma. Symptoms of asthma include, but are not limited to, recurrent episodes of shortness of breath (dyspnea), wheezing, chest tightness and cough. Particularly amenable to such an approach are those tissues or cells that are protected from the increase in airway sensitivity seen when challenged by an allergen by reducing or blocking the expression of the integrin αᵥβ₆. Methods according to this aspect of the invention comprise, for example, (a) identifying a patient with asthma or asthma-related symptoms (such as recurrent episodes of shortness of breath, wheezing, chest tightness and cough) and (b) treating the patient with one or more αᵥβ₆-binding ligands, such as one or more αᵥβ₆-binding antibodies or fragments thereof. Methods according to this aspect of the invention further comprise, for example, (a) identifying a patient with increased susceptibility to asthma or asthma-related symptoms, and (b) treating the patient with one or more αᵥβ₆-binding ligands, such as one or more αᵥβ₆-binding antibodies or fragments thereof.

In certain embodiments, the present invention is directed to methods of treating a mammal having or at risk of having symptoms of asthma related conditions. Asthma related conditions include, but are not limited to, fibrosis in epithelial organs, acute lung injury, rhinitis, anaphylaxis, sinusitis, hay fever, allergies, vocal cord dysfunction and gastgroespohageal reflux disease. Particularly amenable to such an approach are those tissues or cells that are protected from the increase in airway sensitivity seen when challenged by an allergen by reducing or blocking the expression of the integrin αᵥβ₆. Methods according to this aspect of the invention comprise, for example, (a) identifying a patient having or at risk of having asthma related conditions (such as fibrosis in epithelial organs, acute lung injury, rhinitis, anaphylaxis, sinusitis, hay fever, allergies, vocal cord dysfunction and gastgroespohageal reflux disease) and (b) treating the patient with one or more αᵥβ₆-binding ligands, such as one or more αᵥβ₆-binding antibodies or fragments thereof. Methods according to this aspect of the invention further comprise, for example, (a) identifying a patient with increased susceptibility to asthma-related conditions, and (b) treating the patient with one or more αᵥβ₆-binding ligands, such as one or more αᵥβ₆-binding antibodies or fragments thereof.

### Methods Comprising Additional Active Agents

In certain embodiments, the methods of the present invention can be used to treat a mammal having or at risk of having one or more symptoms of asthma or an asthma related condition, comprising co-administering to the mammal a therapeutically effective dose of a ligand to the integrin αvβ6 and one or more additional active agents, such as those disclosed throughout U.S. Patent Publication No. 2005/0148562, the disclosure of which is herein incorporated by reference in its entirety. Exemplary additional active agents include, but are not limited to, additional antihistamines (including H1, H3 and H4 receptor antagonists), steroids (e.g., safe steroids), leukotriene antagonists, prostaglandin D2 receptor antagonists, decongestants, expectorants, anti-fungal agents, triamcinolone and triamcinolone derivatives, non-steroidal immunophilin-dependent immunosuppressants (NsIDIs), anti-inflammatory agents, non-steroidal anti-inflammatory agents (NSAIDs), COX-2 inhibitors, anti-infective agents, mucolytic agents, anticholinergic agents, mast cell stabilizers, non-antibiotic anti-microbial agents, anti-viral agents, antiseptics, neurokinin antagonists, platelet activating factor (PAF) and 5-lipoxygenase (5-LO) inhibitors.

Thus, it is contemplated that the treatment methods of the present invention may be used as a combination therapy wherein a composition comprising one or more antibody or antibody fragment have binding specificity for αvβ6 integrins is administered in combination with one or more other medicaments used for controlling asthma. There are two major groups of medications used in controlling asthma—anti-inflammatories (corticosteroids) and bronchodilators. Anti-inflammatory medicaments reduce the number of inflammatory cells in the airways and prevent blood vessels from leaking fluid into the airway tissues. By reducing inflammation, they reduce the spontaneous spasm of the airway muscle. Anti-inflammatories are used as a preventive measure to lessen the risk of acute asthma attacks.

Examples of antihistamines suitable for inclusion in the present methods include, but are not limited to, acrivastine, azelastine, cyclizine, carebastine, cyproheptadine, carbinoxamine, doxylamine, dimethindene, ebastine, epinastine, efletirizine, ketotifen, levocabastine, mizolastine, mequitazine, mianserin, noberastine, meclizine, norastemizole, olopatadine, picumast, tripelenamine, temelastine, trimeprazine, triprolidine, bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine, pyrilamine, astemizole, terfenadine, loratadine, cetirizine, levocetirizine, fexofenadine, descarboethoxyloratadine, desloratadine, dimenhydrinate and hydroxyzine.

Examples of H3 receptor antagonists suitable for inclusion in the present methods include, but are not limited to, thioperamide, impromidine, burimamide, clobenpropit, impentamine, mifetidine, clozapine, S-sopromidine, R-sopromidine and ciproxifam.

Exemplary anti-inflammatory medicaments for the treatment of asthma include leukotriene inhibitors. Zafirlukast (Accolate), montelukast (Singulair) and zileuton (Zyflo) belong to this class of agents. These drugs are administered orally and inhibit leukotrienes from binding to smooth muscle cells lining the airways. Other inhaled anti-inflammatory drugs include cromolyn sodium (Intal) and nedrocromil (Tilade).

Examples of leukotriene antagonists (e.g., leukotriene D4 antagonists) suitable for inclusion in the present methods include, but are not limited to, albuterol sulfate, aminophylline, amoxicillin, ampicillin, astemizole, attenuated tubercle bacillus, azithromycin, bacampicillin, beclomethasone dipropionate, budesonide, bupropion hydrochloride, cefaclor, cefadroxil, cefixime, cefprozil, cefuroxime axetil, cephalexin, ciprofloxacin hydrochloride, clarithromycin, clindamycin, cloxacillin, doxycycline, erythromycin, ethambutol, fenoterol hydrobromide, fluconazole, flunisolide, fluticasone propionate, formoterol fumarate, gatifloxacin, influenza virus vaccine, ipratropium bromide, isoniazid, isoproterenol hydrochloride, itraconazole, ketoconazole, ketotifen, levofloxacin, minocycline, montelukast (e.g., montelukast sodium), moxifloxacin, nedocromil sodium, nicotine, nystatin, ofloxacin, orciprenaline, oseltamivir, oseltamivir sulfate, oxtriphylline, penicillin, pirbuterol acetate, pivampicillin, pneumococcal conjugate vaccine, pneumococcal polysaccharide vaccine, prednisone, pyrazinamide, rifampin, salbutamol, salmeterol xinafoate, sodium cromoglycate (cromolyn sodium), terbutaline sulfate, terfenadine, theophylline, triamcinolone acetonide, zafirlukast and zanamivir.

Examples of decongestants suitable for inclusion in the present methods include, but are not limited to, pseudoephedrine, phenylephedrine, phenylephrine, phenylpropanolamine, oxymetazoline, propylhexedrine, xylometazoline, epinephrine, ephedrine, desoxyephedrine, naphazoline, and tetrahydrozoline.

Examples of expectorants suitable for inclusion in the present methods include, but are not limited to, guaifenesin, codeine phosphate, and isoproternol hydrochloride.

Examples of anti-fungal agents suitable for inclusion in the present methods include, but are not limited to, amphotericin B, nystatin, fluconazole, ketoconazole, terbinafine, itraconazole, imidazole, triazole, ciclopirox, clotrimazole, and miconazole.

Examples of NSAIDs suitable for inclusion in the present methods include, but are not limited to, ibuprofen, aceclofenac, diclofenac, naproxen, etodolac, flurbiprofen, fenoprofen, ketoprofen, suprofen, fenbufen, fluprofen, tolmetin sodium, oxaprozin, zomepirac, sulindac, indomethacin, piroxicam, mefenamic acid, nabumetone, meclofenamate sodium, diflunisal, flufenisal, piroxicam, ketorolac, sudoxicam and isoxicam.

By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs suitable for inclusion in the present compositions include, but are not limited to, calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which bind to an FK506-binding protein, FKBP-12, and block antigen-induced proliferation of white blood cells and cytokine secretion.

Examples of COX-2 inhibitors suitable for inclusion in the present methods include, but are not limited to, rofecoxib, celecoxib, valdecoxib, lumiracoxib, meloxicam, and nimesulide.

Corticosteroid anti-inflammatory agents are administered in two ways—inhaled via a metered dose inhaler (MDI) or orally via pill/tablet or liquid form. Examples of inhaled corticosteroids include fluticasone (Flovent), budesonide (Pulmicort), flunisolide (AeroBid), triamcinolone (Azmacort, Nasacort, Atlone) and beclomethasone (Beclovent, Vaceril and Vancenase). Examples of oral corticosteroids (pill/tablet form) are prednisone (Deltasone, Meticorten or Paracort), methylprednisolone (Medrol) and prednisolone (Delta Cortef and Sterane). The oral corticosteroids (liquid form) are Pedipred and Prelone. These liquid forms are used for asthmatic children. Pediatric therapies for the treatment of asthma are particularly contemplated. Additional examples of steroids suitable for inclusion in the present methods include but are not limited to, fluoromethalone, fluticasone, mometasone, triamcinolone, betamethasone, flunisolide, budesonide, beclomethasone, budesonide, rimexolone, beloxil, prednisone, loteprednol, dexamethasone and its analogues (e.g., dexamethasone beloxil) described in U.S. Pat. Nos. 5,223,493 and 5,420,120, incorporated herein by reference in their entireties.

Bronchodilators work by increasing the diameter of the air passages and easing the flow of gases to and from the lungs. They come in two basic forms—short-acting and long-acting. Examples of short-acting bronchodilators include metaproterenol (Alupent, Metaprel), ephedrine, terbutaline (Brethaire) and albuterol (Proventil, Ventolin). These drugs are inhaled and are used to relieve symptoms during acute asthma attacks. Examples of long-acting bronchodilators include salmeterol (Serevent), metaproterenol (Alupent) and theophylline (Aerolate, Bronkodyl, Slo-phyllin, and Theo-Dur) and aminophylline. Serevent and Alupent are inhaled and theophylline is taken orally. Theophylline and aminophylline are examples of methylxanthine medications. This group of medications is chemically related to caffeine and has frequently been used in the routine management of asthma.

Anticholinergics are another class of drugs useful as rescue medications during asthma attacks. Inhaled anticholinergic drugs open the breathing passages, similar to the action of the beta-agonists. Inhaled anticholinergics take slightly longer than beta-agonists to achieve their effect, but they last longer than the beta-agonists. An anticholinergic drug is often used together with a beta-agonist drug to produce a greater effect than either drug can achieve by itself. Ipratropium bromide (Atrovent) is an inhaled anticholinergic drug commonly used as a rescue asthma medication. Advair is another inhaled medication that combines fluticasone and salmeterol to reduce both inflammation and airway constriction.

Other medications focus on treating allergy triggers for asthma and include: immunotherapy and anti-IGE monoclonal antibodies. Immunotherapy-based treatment of asthma involves allergy-desensitization series of therapeutic injections containing small doses of allergens to desensitize the subject to the allergen in question. Another therapy for allergic asthma involves treatment with anti-IgE antibodies as exemplified by omalizumab (Xolair). Xolair is used in children over 12 years old and adults with moderate to severe asthma caused by an allergy.

Examples of anti-infective agents suitable for inclusion in the present methods include, but are not limited to, penicillins and other beta lactam antibiotics, cephalosporins, macrolides, ketolides, sulfonamides, quinolones, aminoglycosides, and linezolid.

Examples of non-antibiotic antimicrobials suitable for inclusion in the present methods include, but are not limited to, taurolidine.

Examples of mast cell stabilizers suitable for inclusion in the present methods include, but are not limited to, cromolyn and nedcromil sodium.

Examples of mucolytic agents suitable for inclusion in the present methods include, but are not limited to, acetylcysteine and dornase alpha.

Examples of antibiotic agents suitable for inclusion in the present methods include, but are not limited to, cefuroxime, vancomycin, amoxicillin and gentamicin.

Examples of antiseptics suitable for inclusion in the present methods include, but are not limited to, iodine, chlorhexidine acetate, sodium hypochlorite, and calcium hydroxide.

Examples of anticholinergics suitable for inclusion in the present methods include, but are not limited to, ipratropium, atropine, and scopolamine.

Examples of neurokinin antagonists suitable for inclusion in the present methods include, but are not limited to, oximes, hydrazones, piperidines, piperazines, aryl alkyl amines, hydrazones, nitroalkanes, amides, isoxazolines, quinolines, isoquinolines, azanorbornanes, naphthyridines, and benzodiazepines, such as those disclosed in U.S. Pat. Nos. 5,798,359; 5,795,894; 5,789,422; 5,783,579; 5,719,156; 5,696,267; 5,691,362; 5,688,960; 5,654,316, incorporated by reference herein in their entireties.

Examples of 5-lipoxygenase (5-LO) inhibitors suitable for inclusion in the present methods include, but are not limited to, zileuton, docebenone, piripost and tenidap.

### Diagnostic Kits

In additional embodiments, the present invention provides kits, particularly kits useful in treatment or prevention of diseases or disorders such as asthma. Kits according to this aspect the present invention may comprise at least one container containing one or more of the above-described ligands, such as antibodies, that bind to or recognize integrin αvβ6. These kits of the invention may optionally further comprise at least one additional container which may contain, for example, a reagent (such as a buffered salt solution) for delivering the ligand (e.g., antibody) to a test sample such as an organ, tissue or cell sample from a patient. Other suitable additional components of such kits of the invention will be familiar to those of ordinary skill in the art.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### Examples

In the present experiments, we set out to study mice expressing a null mutation of the integrin β6 subunit under chronic allergen challenge. Our data support a role in human asthma for αᵥβ₆ and suggest that therapeutic intervention using a function-blocking αᵥβ₆ mAb would be a valuable method for treating, controlling and/or preventing asthma.

### Sensitization and Challenge

Six to eight week old sex-matched C57BL/6 wild-type and β6 knockout mice were sensitized intraperitoneally on days 0 and 12 with 50 μg of OVA (grade V; Sigma-Aldrich, St. Louis, Mo., USA) adsorbed to 1 mg of alum (Sigma-Aldrich) in 200 μl normal saline. Intranasal OVA challenges (20 ng/50 μl in saline) were administered on days 26, 29 and 32 under isoflurane anesthesia and then repeated twice a week for 7 weeks. A higher dose OVA challenge (1 mg/50 ul in saline) was performed for another 7 weeks. 24 hours after the last challenge, mice were analyzed for lung mechanics and lung inflammation.

### Measurement of Airway Response to Acetylcholine

Mice were anesthetized with Ketamine (100 mg/kg) and Xylazine (10 mg/kg). A tracheostomy was performed and a tubing adaptor (20 gauge) was used to cannulate the trachea. The mice were then attached to a rodent ventilator and pulmonary mechanics analyzer (FlexiVent, SIRAQ Inc, Canada) and ventilated at a tidal volume of 9 ml/kg, a frequency of 150 breaths/minute and 2 cm H2O positive end-expiratory pressure. Mice were paralyzed with pancuronium (0.1 mg/kg intraperitoeally). A 27 G needle was placed in the tail vein and measurements of airway mechanics were made continuously with a Sinusoidal signal at a single frequency. Mice were given increasing doses of acetylcholine (0.03, 0.1, 0.3, 1 and 3 μg/g body weight) administered through the tail vein to generate a concentration-response curve.

### Assessment of Airway Inflammation and Mucus Production.

Lungs were lavaged 5 times with 0.8 ml of PBS. After centrifugation (1000 rpm, 5 min), the cell pellet was resuspended in normal saline after lysis of red blood cells. Total cells were counted with a hemacytometer. Cytospin preparations were prepared and stained with HEMA 3 stain set (Fisher), and bronchoalveolar lavage (BAL) fluid cell differential percentages were determined based on light microscopic evaluation of >300 cells/slide.

After lavage, lungs were inflated with 10% buffered formalin to 25 cmH₂O of pressure and transferred into tubes containing 10% buffered formalin. Multiple paraffin-embedded 5-μm sections of the entire mouse lung were prepared and stained with hematoxylin and eosin (H&E) for regular morphology and with periodic acid-Schiff (PAS) for evaluation of mucus production.

### Quantification of Peribronchial Fibrosis and Smooth Muscle

The areas of peribronchial Sirius-red and α-smooth muscle actin staining in a paraffin-embedded lung were outlined and quantified using a light microscope attached to a Computer-Assisted Stereology Toolbox software system (C.A.S.T-Grid; Olympus, Albertslund, Denmark). A blinded operator measured the total lung volume and the volume of Sirius-red or α-smooth muscle actin positive area by point counting of randomly sampled microscopic fields. At least ten bronchioles were counted in each slide.

### Results

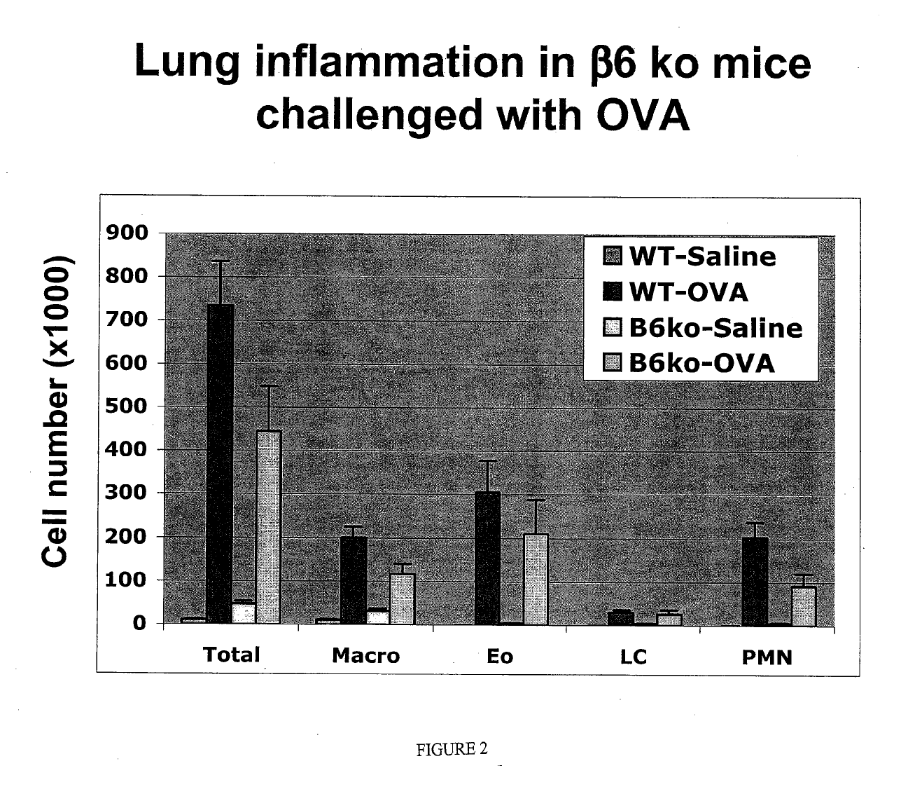

In order to measure lung inflammation, the total number of cells were counted in wild-type mice challenged with saline and wild-type mice challenged with ovalalbumin (OVA). In addition, cell numbers were counted for β6 knockout mice challenged with saline and β6 knockout mice challenged with OVA. Cell numbers were counted for total cells, macrophages, eosinophils, leukocytes and polymorphonuclear leukocytes. The β6 knockout mice that were challenged with OVA showed a decrease in total cells, macrophages, eosinophils, leukocytes and polymorphonuclear leukocytes as compared to wild-type mice challenged with OVA. The results are shown in FIG. 2.

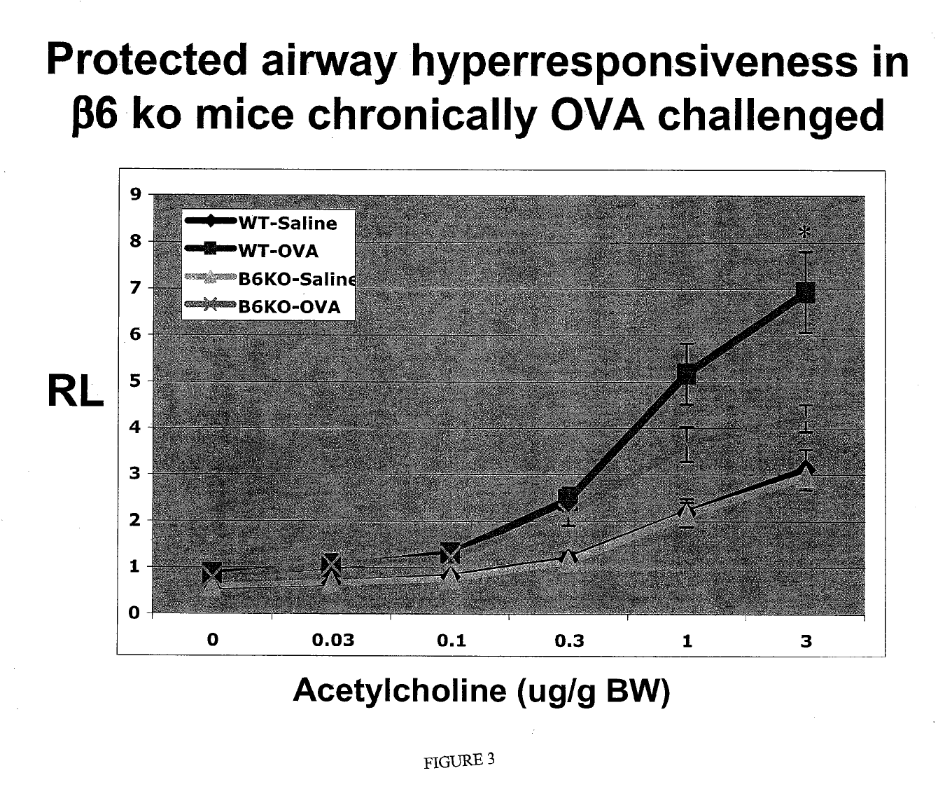

Mice were given increasing doses of acetylcholine (0.03, 0.1, 0.3, 1 and 3 μg/g body weight) administered through the tail vein to generate a concentration-response curve. A concentration-response curve was measured for wild-type mice challenged with saline and wild-type mice challenged with OVA, along with β6 knockout mice challenged with saline and β6 knockout mice challenged with OVA. The results are shown in FIG. 3. These results show that β6 knockout mice have significantly less responsiveness to acetylcholine-induced bronchoconstriction after chronic allergen challenge than do control wild type mice.

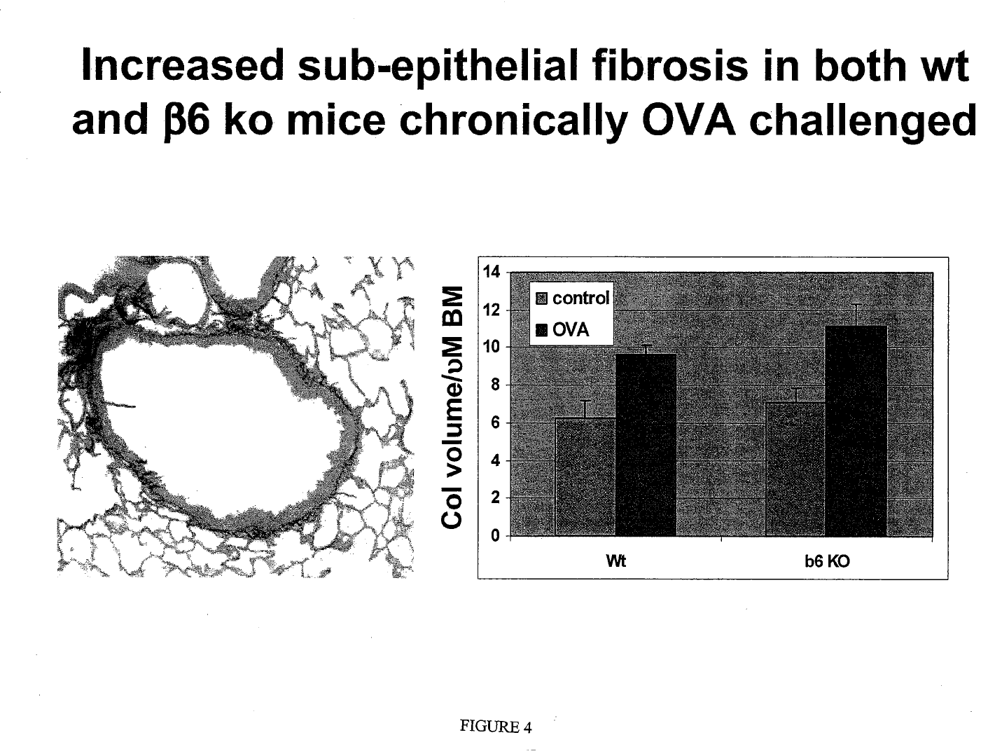

In addition, both wild-type and β6 knockout mice challenged with OVA show an increase in sub-epithelial fibrosis. The results are shown in FIG. 4. These results show no difference in sub-epithelial fibrosis between β6 knockout and wild type mice, demonstrating that protection from sub-epithelial fibrosis is not responsible for the protection of the knockout mice from induced airway hyperresponsiveness.

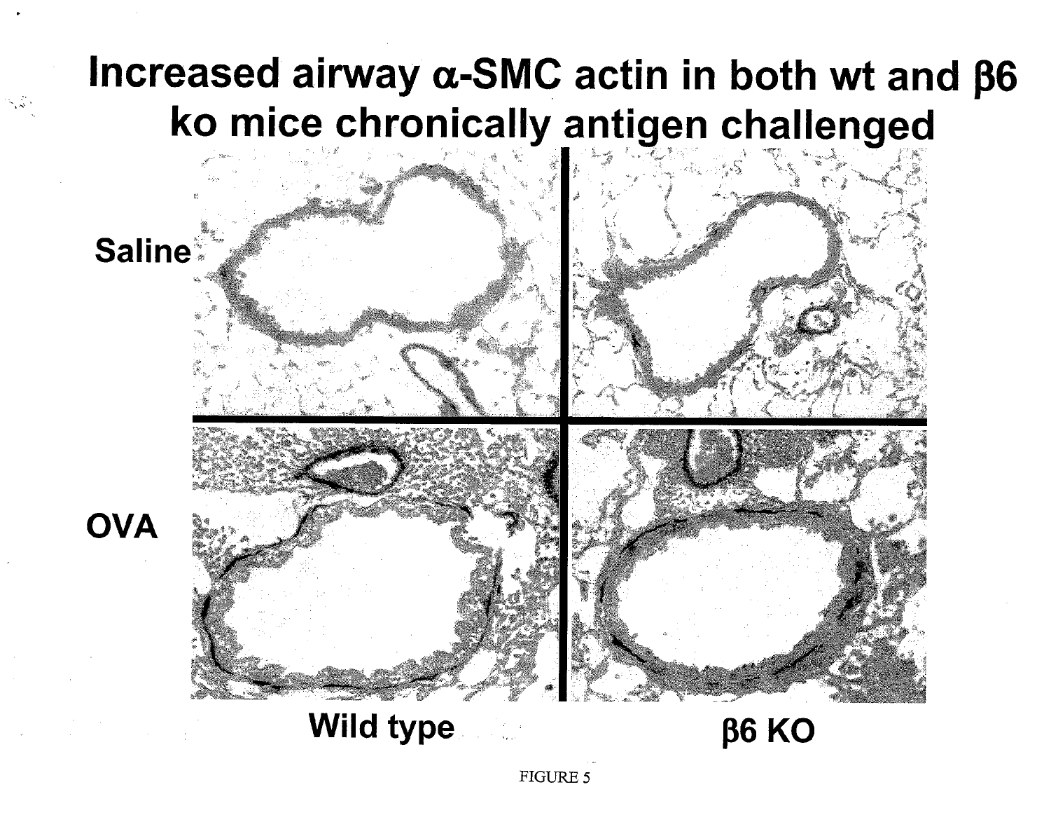

Both wild-type and β6 knockout mice challenged with OVA showed an increase in airway α-SMC actin as compared to both wild-type and β6 knockout mice challenged with saline. The results are shown in FIG. 5. These results demonstrate similar increases in smooth muscle volume in β6 knockout and wild type mice in response to chronic allergen challenge, suggesting that protection from allergen-induced smooth muscle hyperplasia is not responsible for the protection of the knockout mice from induced airway hyperresponsiveness.

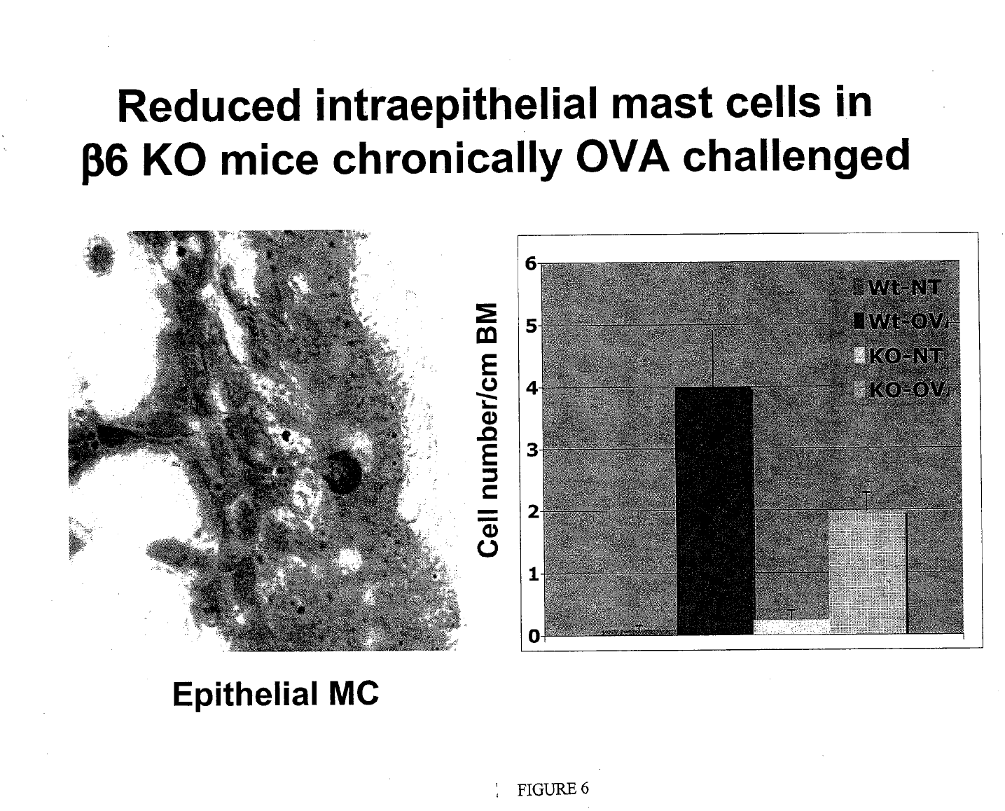

β6 knockout mice challenged with OVA show a reduced number of intraepithelial mast cells when compared to wild-type mice challenged with OVA. The results are shown in FIG. 6. The reduction in epithelial mast cells seen in allergen-challenged β6 knockout mice might explain the protection from airway hyperresponsiveness seen in these animals.

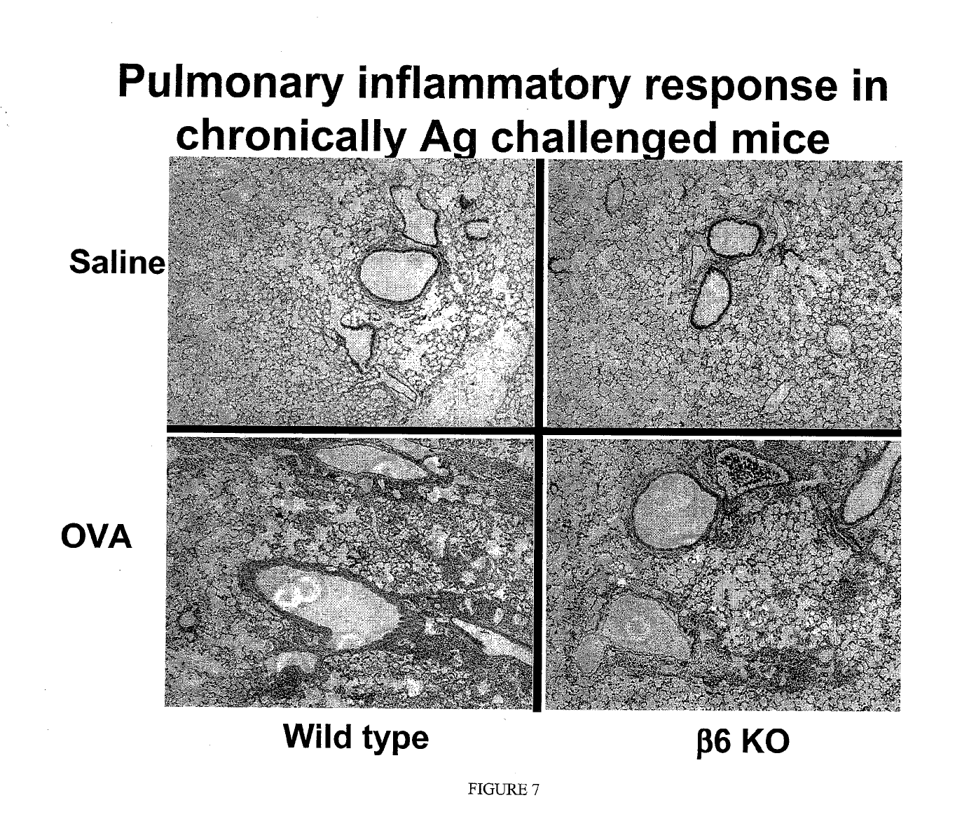

The pulmonary inflammatory response in both wild-type and β6 knockout mice challenged with OVA versus both wild-type and β6 knockout mice challenged with saline is shown if FIG. 7. There are no differences in pulmonary inflammation in response to chronic allergen challenge in β6 knockout and wild type mice. These results suggest that general protection from the inflammatory response to chronic allergen challenge does not explain the protection from airway hyperresponsiveness seen in β6 knockout mice.

Having now fully described this invention, it will be understood to those of ordinary skill in the art that the same can be performed within a wide and equivalent range of conditions, formulations, and other parameters without affecting the scope of the invention or any embodiment thereof.

All documents, e.g., scientific publications, patents, patent applications and patent publications recited herein are hereby incorporated by reference in their entirety to the same extent as if each individual document was specifically and individually indicated to be incorporated by reference in its entirety. Where the document cited only provides the first page of the document, the entire document is intended, including the remaining pages of the document.

1-169. (canceled)

170. A method of treating a mammal having or at risk of having asthma, comprising administering to the mammal a therapeutically effective dose of an antibody or a fragment thereof wherein said antibody or fragment thereof is derived from an antibody produced by hybridoma 6.2A1 (ATCC accession number PTA-3896), an antibody produced by hybridoma 6.2E5 (ATCC accession number PTA-3897), an antibody produced by hybridoma 6.1A8 (ATCC accession number PTA-3647), an antibody produced by hybridoma 6.2B10 (ATCC accession number PTA-3648), an antibody produced by hybridoma 6.2B1 (ATCC accession number PTA-3646), an antibody produced by hybridoma 7.1G10 (ATCC accession number PTA-3898), an antibody produced by hybridoma 7.7G5 (ATCC accession number PTA-3899), an antibody produced by hybridoma 7.1C5 (ATCC accession number PTA-3900), an antibody produced by hybridoma 6.8G6 (ATCC accession number PTA-3645), or an antibody produced by hybridoma 6.3G9 (ATCC accession number PTA-3649).

171. The method of claim 170 wherein said antibody of antibody fragment thereof is administered in a dose effective to produce a reduction in epithelial mast cells in the lungs of said mammal.

172. The method of claim 170, wherein said antibody is a monoclonal antibody that is a chimeric, primatized or humanized monoclonal antibody.

173. The method of claim 170, wherein said monoclonal antibody is selected from the group consisting of hybridoma 6.2A1 (ATCC accession number PTA-3896), an antibody produced by hybridoma 6.2E5 (ATCC accession number PTA-3897), an antibody produced by hybridoma 6.1A8 (ATCC accession number PTA-3647), an antibody produced by hybridoma 6.2B10 (ATCC accession number PTA-3648), an antibody produced by hybridoma 6.2B1 (ATCC accession number PTA-3646), an antibody produced by hybridoma 7.1G10 (ATCC accession number PTA-3898), an antibody produced by hybridoma 7.7G5 (ATCC accession number PTA-3899), an antibody produced by hybridoma 7.1C5 (ATCC accession number PTA-3900), as an antibody produced by hybridoma 6.8G6 (ATCC accession number PTA-3645), an antibody produced by hybridoma 6.3G9 (ATCC accession number PTA-3649).

174. The method of claim 172, wherein the humanized antibody comprises heavy and light chain variable domains of SEQ ID NO:1 and SEQ ID NO:2, respectively.

175. The method of claim 172, wherein the humanized monoclonal antibody comprises a heavy chain whose CDR 1, 2 and 3 comprise amino acids 31-35, 50-65 and 98-109 of SEQ ID NO:1, respectively and whose light chain CDR 1, 2 and 3 comprise amino acids 24-35, 51-57 and 90-98, respectively of SEQ ID NO:2, respectively.

176. The method of claim 172, wherein the humanized monoclonal antibody comprises a heavy chain whose framework regions (FR) 1, 2, 3 and 4 comprise amino acid residues 1-30, 36-49, 66-97 and 110-120 of SEQ ID NO: 1, respectively;
a heavy chain whose CDR 1, 2 and 3 comprise amino acids 31-35, 50-65 and 98-109 of SEQ ID NO:1, respectively and
whose light chain CDR 1, 2 and 3 comprise amino acids 24-35, 51-57 and 90-98, respectively of SEQ ID NO:2, respectively; and
a light chain whose framework regions (FR) 1, 2, 3 and 4 comprise amino acid residues 1-23, 36-50, 58-89 and 99-108, respectively, of SEQ ID NO: 2.

177. The method of claim 172, wherein the humanized monoclonal antibody comprises a heavy chain version selected from the group consisting of heavy chain version 1 (“HV1”) comprising a sequence of SEQ ID NO:3; heavy chain version 2 (“HV2”) comprising a sequence of SEQ ID NO:56, and heavy chain version 3, (“HV3”) comprising a sequence of SEQ ID NO:57.

178. The method of claim 172, wherein the humanized monoclonal antibody comprises a light chain version selected from the group consisting of light chain version 1 (“LV1”), light chain version 2 (“LV2”), light chain version 3 (“LV3”), light chain version 4 (“LV4”) and light chain version 5 (“LV5”), wherein LV1 light chain consists of amino acid substitutions L47W, 158 V, A60V and Y87F of SEQ ID NO: 2; the LV2 light chain consists of amino acid substitutions L47W and 158V of SEQ ID NO: 2; the LV3 light chain consists of amino acid substitution L47W of SEQ ID NO: 2; the LV4 light chain consists of amino acid substitutions EIQ and L47W of SEQ ID NO: 2 and the LV5 light chain consists of SEQ ID NO: 2.

179. The method of claim 172, wherein the humanized monoclonal antibody comprises:
a) a heavy chain CDR1 that comprises a sequence selected from the group consisting of any one of SEQ ID NOs 101-105;
b) a heavy chain CDR2 that comprises a sequence selected from the group consisting of any one of SEQ ID NOs 106-111;
c) a heavy chain CDR3 that comprises a sequence selected from the group consisting of any one of SEQ ID NOs 112-117.

180. The method of claim 172, wherein the humanized monoclonal antibody comprises:
a) a light chain CDR1 that comprises a sequence selected from the group consisting of any one of SEQ ID NOs: 118-123;
b) a light chain CDR2 that comprises a sequence selected from the group consisting of any one of SEQ ID NOs:124-127; and
c) a light chain CDR3 that comprises a sequence selected from the group consisting of any one of SEQ ID NOs 128-133.

181. A method of treating asthma comprising administering an antibody or a fragment thereof which (36 subunit of the integrin αvβ6 in the αvβ6 complex but does not bind αv alone wherein said antibody or fragment thereof is derived from an antibody produced by hybridoma 6.2A1 (ATCC accession number PTA-3896), an antibody produced by hybridoma 6.2E5 (ATCC accession number PTA-3897), an antibody produced by hybridoma 6.1A8 (ATCC accession number PTA-3647), an antibody produced by hybridoma 6.2B10 (ATCC accession number PTA-3648), an antibody produced by hybridoma 6.2B1 (ATCC accession number PTA-3646), an antibody produced by hybridoma 7.1G10 (ATCC accession number PTA-3898), an antibody produced by hybridoma 7.7G5 (ATCC accession number PTA-3899), an antibody produced by hybridoma 7.1C5 (ATCC accession number PTA-3900), an antibody produced by hybridoma 6.8G6 (ATCC accession number PTA-3645), or an antibody produced by hybridoma 6.3G9 (ATCC accession number PTA-3649).

182. The method of claim 170 further comprising administering a therapeutically effective dose of one or more additional active agents for the treatment of asthma.

183. A method of alleviating edema in the lung airways of an animal comprising administering to said animal a therapeutically effective dose of an antibody or a fragment thereof that binds to one or more subunits of the integrin αvβ6, wherein said antibody is a blocking antibody wherein said blocking antibody is administered in a dose effective to produce a reduction in epithelial mast cells in the lungs of said mammal wherein said antibody or fragment thereof is derived from an antibody produced by hybridoma 6.2A1 (ATCC accession number PTA-3896), an antibody produced by hybridoma 6.2E5 (ATCC accession number PTA-3897), an antibody produced by hybridoma 6.1A8 (ATCC accession number PTA-3647), an antibody produced by hybridoma 6.2B10 (ATCC accession number PTA-3648), an antibody produced by hybridoma 6.2B1 (ATCC accession number PTA-3646), an antibody produced by hybridoma 7.1G10 (ATCC accession number PTA-3898), an antibody produced by hybridoma 7.7G5 (ATCC accession number PTA-3899), an antibody produced by hybridoma 7.1C5 (ATCC accession number PTA-3900), an antibody produced by hybridoma 6.8G6 (ATCC accession number PTA-3645), or an antibody produced by hybridoma 6.3G9 (ATCC accession number PTA-3649).

184. A method of decreasing mucus production in the lung airways of an animal comprising administering to said animal a therapeutically effective dose of an antibody or a fragment thereof that binds to integrin αvβ6 wherein said antibody is administered in a dose effective to produce a reduction in epithelial mast cells in the lungs of said animal and wherein said antibody or fragment thereof is derived from an antibody produced by hybridoma 6.2A1 (ATCC accession number PTA-3896), an antibody produced by hybridoma 6.2E5 (ATCC accession number PTA-3897), an antibody produced by hybridoma 6.1A8 (ATCC accession number PTA-3647), an antibody produced by hybridoma 6.2B10 (ATCC accession number PTA-3648), an antibody produced by hybridoma 6.2B1 (ATCC accession number PTA-3646), an antibody produced by hybridoma 7.1G10 (ATCC accession number PTA-3898), an antibody produced by hybridoma 7.7G5 (ATCC accession number PTA-3899), an antibody produced by hybridoma 7.1C5 (ATCC accession number PTA-3900), an antibody produced by hybridoma 6.8G6 (ATCC accession number PTA-3645), or an antibody produced by hybridoma 6.3G9 (ATCC accession number PTA-3649).

185. A method of decreasing epithelial denudation of lung tissue in an animal comprising administering to said animal a therapeutically effective dose of an antibody or a fragment thereof wherein said antibody or fragment thereof is derived from an antibody produced by hybridoma 6.2A1 (ATCC accession number PTA-3896), an antibody produced by hybridoma 6.2E5 (ATCC accession number PTA-3897), an antibody produced by hybridoma 6.1A8 (ATCC accession number PTA-3647), an antibody produced by hybridoma 6.2B10 (ATCC accession number PTA-3648), an antibody produced by hybridoma 6.2B1 (ATCC accession number PTA-3646), an antibody produced by hybridoma 7.1G10 (ATCC accession number PTA-3898), an antibody produced by hybridoma 7.7G5 (ATCC accession number PTA-3899), an antibody produced by hybridoma 7.1C5 (ATCC accession number PTA-3900), an antibody produced by hybridoma 6.8G6 (ATCC accession number PTA-3645), or an antibody produced by hybridoma 6.3G9 (ATCC accession number PTA-3649) and is administered in a dose effective to produce a reduction in epithelial mast cells in the lungs of said animal.

186. A method of alleviating one or more of the symptoms of an asthma-related condition selected from the group consisting of fibrosis of epithelial tissue of the lung, acute lung injury, rhinitis, anaphylaxis, sinusitis, hay fever, vocal cord dysfunction and gastroesophageal reflux disease in an animal comprising administering to said animal a therapeutically effective dose of an antibody or a fragment thereof that binds integrin αvβ6, wherein said antibody or fragment thereof is derived from an antibody produced by hybridoma 6.2A1 (ATCC accession number PTA-3896), an antibody produced by hybridoma 6.2E5 (ATCC accession number PTA-3897), an antibody produced by hybridoma 6.1A8 (ATCC accession number PTA-3647), an antibody produced by hybridoma 6.2B10 (ATCC accession number PTA-3648), an antibody produced by hybridoma 6.2B1 (ATCC accession number PTA-3646), an antibody produced by hybridoma 7.1G10 (ATCC accession number PTA-3898), an antibody produced by hybridoma 7.7G5 (ATCC accession number PTA-3899), an antibody produced by hybridoma 7.1C5 (ATCC accession number PTA-3900), an antibody produced by hybridoma 6.8G6 (ATCC accession number PTA-3645), or an antibody produced by hybridoma 6.3G9 (ATCC accession number PTA-3649) and is administered in a dose effective to produce a reduction in epithelial mast cells in the lungs of said animal.

187. A method of treating a mammal having or at risk of having one or more symptoms of asthma, comprising co-administering to the mammal:
a) a therapeutically effective dose of an antibody or a fragment thereof wherein said antibody or fragment thereof is derived from an antibody produced by hybridoma 6.2A1 (ATCC accession number PTA-3896), an antibody produced by hybridoma 6.2E5 (ATCC accession number PTA-3897), an antibody produced by hybridoma 6.1A8 (ATCC accession number PTA-3647), an antibody produced by hybridoma 6.2B10 (ATCC accession number PTA-3648), an antibody produced by hybridoma 6.2B1 (ATCC accession number PTA-3646), an antibody produced by hybridoma 7.1G10 (ATCC accession number PTA-3898), an antibody produced by hybridoma 7.7G5 (ATCC accession number PTA-3899), an antibody produced by hybridoma 7.1C5 (ATCC accession number PTA-3900), an antibody produced by hybridoma 6.8G6 (ATCC accession number PTA-3645), or an antibody produced by hybridoma 6.3G9 (ATCC accession number PTA-3649) and wherein said dose is an amount effective to produce a reduction in epithelial mast cells in the lungs of said animal and
b) one or more additional active agents.
